(19) United States
(12) Patent Application Publication
(10) Pub. No.: US 2026/0201023 A1
(43) Pub. Date: Jul. 16, 2026

(54) ANTI-TAU MTBR ANTIBODIES AND METHODS TO DETECT CLEAVED FRAGMENTS OF TAU AND USES THEREOF

(71) Applicant: Washington University, St. Louis, MO (US)

(72) Inventors: Randall Bateman, St. Louis, MO (US); David Holtzman, St. Louis, MO (US); Kanta Horie, St. Louis, MO (US); Hong Jiang, St. Louis, MO (US)

(21) Appl. No.: 19/102,735

(22) PCT Filed: Aug. 23, 2023
(86) PCT No.: PCT/US23/72738

§ 371 Date: Feb. 10, 2025

Related U.S. Application Data
(60) Provisional application No. 63/400,345, filed on Aug. 23, 2022.
(60) Provisional application No. 63/443,317, filed on Feb. 3, 2023.
(60) Provisional application No. 63/452,291, filed on Mar. 15, 2023.

(51) Int. Cl. C07K 16/18; G01N 33/68

(52) U.S. Cl. CPC C07K 16/18; G01N 33/6896; G01N 2333/95; G01N 2800/2821; G01N 2800/52

(57) ABSTRACT

Provided herein are antibodies, or fragments thereof, that specifically bind to a microtubule-binding region (MTBR) of tau, and uses thereof. Further provided are methods of detecting species of MTBR in blood or cerebral spinal fluid, and the use of such detection for diagnosing, prognosing, or staging pathological features and/or clinical symptoms of tauopathies, and to choose treatments appropriate for a given disease stage.

### FIELD OF THE INVENTION

The present invention relates to antibodies, or fragments thereof, that specifically bind to a microtubule-binding region (MTBR) of tau, and uses thereof. The invention further relates to methods of detecting mature neurofibrillary tangles in patients with tauopathies. The invention also relates to detecting species of MTBR in blood or cerebral spinal fluid, and the use of such detection for diagnosing, prognosing, or staging pathological features and/or clinical symptoms of tauopathies, and to choose treatments appropriate for a given disease stage.

### BACKGROUND OF THE INVENTION

Accumulation of tau protein as insoluble aggregates in the brain is one of the hallmarks of Alzheimer's disease and other neurodegenerative diseases called tauopathies. Tau pathology appears to propagate across brain regions and spread by the transmission of specific pathological tau species from cell to cell in a prion-like manner although the nature of these species (i.e., monomeric, oligomeric, and fibril species) and the spreading process are uncertain (Frost et al., 2009; Goedert et al., 2010, 2017; Sanders et al., 2014; Wu et al., 2016; Mirbaha et al., 2018; Lasagna-Reeves et al., 2012). Tau has six different isoforms of the full-length protein. In addition, tau has more than one hundred potential post-translational modification sites, including phosphorylation, in addition to multiple truncation sites (Meredith et al., 2013; Sato et al., 2018; Barthélemy et al., 2019; Cicognola et al., 2019; Blennow et al., 2020). Thus, identifying specific pathological tau species involved in tau spread is challenging. Several mass spectrometry (MS) studies suggest that the microtubule-binding region (MTBR) of tau is enriched in aggregates in Alzheimer's disease brain (Taniguchi-Watanabe et al., 2016; Roberts et al., 2020). Moreover, a series of cryogenic electron microscopy (Cryo-EM) studies demonstrate that the core structure of tau aggregates consists of a sub-segment of the MTBR domain and the particular conformation depends on the tauopathy (Fitzpatrick et al., 2017; Falcon et al., 2018, 2019; Zhang et al., 2020). These findings strongly suggest that MTBR tau is critical for tau aggregation. However, these studies used postmortem brain tissue. Little is known about the pathophysiology of corresponding extracellular MTBR-containing tau species in biological samples such as CSF and blood, which may serve as a surrogate biomarker of brain tau aggregates in living humans.

CSF is routinely obtained from study participants via lumbar puncture during clinical visits. Previous CSF tau biomarker studies suggested that MTBR tau was missing in CSF and focused on N-terminal and mid-domain regions (Meredith et al., 2013; Sato et al., 2018). Species composed of the N-terminus to mid-domain appear to be actively secreted from neurons into the extracellular space after truncation between the mid- and the MTBR-domain (Sato et al., 2018). Detection of MTBR tau species were reported (Barthélemy et al., 2016b, a) but have not been characterized in relationship to disease. Recently, a tau species containing a cleavage at residue 368 (tau368) within the repeat region 4 (RF) was identified in CSF (Blennow et al., 2020). It is unclear, however, whether tau368 reflects the overall pool of MTBR tau species given the variations in regions, truncations and conformational structures not captured by antibodies.

Advances in high resolution mass spectrometry techniques have created new methodologies to measure the abundance of proteins in biological samples. Despite advances in instrumentation and data analysis software, sample preparation is still an immense challenge. The choice of sample preparation method affects the observed metabolite profile and data quality, and can ultimately affect reported results. This is particularly true for proteins and peptides in low abundance in biological samples. Peptides that fall under this umbrella include many proteolytic fragments of full-length proteins, which are differentially produced in various disease processes.

Accordingly, there remains a need in the art for improved compositions and methods useful to quantify low abundance, MTBR tau species in biological fluid.

### SUMMARY OF THE INVENTION

Provided herein is an isolated anti-MTBR tau antibody. The isolated anti-MTBR tau antibody may comprise (a) an L1, L2, and L3 from a light chain variable region from the sequence set forth in SEQ ID NO: 6, and an H1, H2, and H3 from a heavy chain variable region from the sequence set forth in SEQ ID NO: 7; (b) an L1, L2, and L3 from a light chain variable region sequence from the sequence set forth in SEQ ID NO: 2, and an H1, H2, and H3 from a heavy chain variable region from the sequence set forth in SEQ ID NO: 3; (c) an L1, L2, and L3 from a light chain variable region from the sequence set forth in SEQ ID NO: 4, and an H1, H2, and H3 from a heavy chain variable region from the sequence set forth in SEQ ID NO: 5; or (d) an L1, L2, and L3 from a light chain variable region from the sequence set forth in SEQ ID NO: 8, and an H1, H2, and H3 from a heavy chain variable region from the sequence set forth in SEQ ID NO: 9. The isolated anti-MTBR tau antibody may comprise (a) a light chain variable region from the sequence set forth in SEQ ID NO: 6 and a heavy chain variable region from the sequence set forth in SEQ ID NO: 7; (b) a light chain variable region from the sequence set forth in SEQ ID NO: 2 and a heavy chain variable region from the sequence set forth in SEQ ID NO: 3; (c) a light chain variable region from the sequence set forth in SEQ ID NO: 4 and a heavy chain variable region from the sequence set forth in SEQ ID NO: 5; or (d) a light chain variable region from the sequence set forth in SEQ ID NO: 8 and a heavy chain variable region from the sequence set forth in SEQ ID NO: 9. The isolated antibody may be a monoclonal antibody or an antibody fragment. The isolated antibody may be a monoclonal antibody.

Provided herein is a method of detecting insoluble tau aggregates in a plasma sample, which may comprise (a) purifying endogenously cleaved fragments of tau by contacting the plasma sample with an epitope-binding agent that specifically binds an epitope within amino acids 235-242 of SEQ ID NO: 1, without first contacting the endogenously cleaved fragments of tau in vitro with a protease; (b) contacting the purified endogenously cleaved fragments of tau with an Arg-C endopeptidase to obtain proteolytic MTBR-tau243 peptides comprising amino acids 243-254 of SEQ ID NO: 1; and (c) detecting the proteolytic MTBR-tau243 peptides by performing liquid chromatography-mass spectrometry (LC/MS) or an immunoassay, wherein detecting the proteolytic MTBR-tau243 peptides is indicative of insoluble tau aggregates in the sample. The purifying step may comprise immunoprecipitation. The proteolytic MTBR-tau243 peptides may comprise one or more of MTBR-tau243-254, MTBR-tau243-256V, MTBR-tau243-256V (dN), and MTBR-tau212-221. The detecting the proteolytic MTBR-tau243 peptides step may comprise performing an immunoassay or liquid chromatography-mass spectrometry. The epitope-binding agent may be an anti-tau antibody comprising a light chain variable region from the sequence set forth in SEQ ID NO: 6 and a heavy chain variable region from the sequence set forth in SEQ ID NO: 7. The anti-tau antibody may be HJ32.11. A solution comprising the proteolytic MTBR-tau243 peptides may be desalted by solid phase extraction before step (c). The method may further comprise one or more of detecting and quantifying one or more of amyloid beta, N-terminal tau, mid-domain tau, post-translational modifications of tau, and an ApoE isoform, in the biological or CSF sample. The quantifying amyloid beta step may comprise quantifying an Aβ42/40 value or quantifying post-translational modifications of tau comprising one or more of phospho-tau217, phospho-tau205, phospho-tau181, phospho-tau153, phospho-tau111, and phospho-tau208.

Provided herein is a method of detecting insoluble tau aggregates in a cerebrospinal fluid (CSF) sample, which may comprise (a) performing affinity depletion on a CSF sample by contacting the sample with affinity depletion agents comprising one or more epitope-binding agents that each binds to one of N-terminal tau, mid-domain tau, and long-MTBR tau, but not to an antigen within amino acids 235-256 of SEQ ID NO: 1, wherein the CSF sample comprises cleaved fragments of tau, to obtain a depleted sample and an enriched sample, wherein the depleted sample comprises N-terminal tau, mid-domain tau, and long-MTBR tau, and wherein the enriched sample is enriched for endogenously cleaved fragments of tau comprising amino acids 235-256 of SEQ ID NO: 1 (endogenous MTBR-tau243 peptides); (b) performing immunoprecipitation on the enriched sample by contacting the enriched sample with an immunoprecipitation agent comprising an epitope-binding agent that binds to endogenous MTBR-tau243 peptides, to obtain a purified sample; (c) contacting the endogenous MTBR-tau243 peptides in the purified sample with a protease to obtain a sample comprising proteolytic MTBR-tau243 peptides; and (d) detecting the proteolytic MTBR-tau243 peptides by performing liquid chromatography-mass spectrometry (LC/MS) or an immunoassay, wherein detecting the proteolytic MTBR-tau243 peptides may be indicative of insoluble tau aggregates in the CSF sample. The sample comprising proteolytic MTBR-tau243 peptides may be desalted before step (d). The affinity depletion agents may comprise: an epitope-binding agent that specifically binds to an epitope within amino acids 1 to 243 of SEQ ID NO: 1; a first epitope-binding agent that specifically binds to an epitope within amino acids 1 to 103 of tau-441, and a second epitope-binding agent that specifically binds to an epitope within amino acids 104 to 243 of SEQ ID NO: 1; or, the first epitope-binding agent, the second epitope-binding agent, and a third epitope-binding agent that specifically binds to an epitope within amino acids 257-264 of SEQ ID NO: 1. The first epitope-binding agent may be HJ8.5, the second epitope-binding agent may be one or more of Tau1 and HJ8.7, and the third epitope-binding agent may be HJ34.8. The affinity depletion agents may comprise Tau1, HJ8.5, and HJ8.7. The affinity depletion agents may further comprise HJ34.8. The immunoprecipitation agent may comprise HJ32.11. The proteolytic MTBR-tau243 peptides may comprise cleaved tau fragments comprising one or more of MTBR-tau243-254, MTBR-tau243-255 (dN), MTBR-tau243-257K (dN), MTBR-tau243-256V, MTBR-tau243-256V (dN), MTBR-tau243-258S, and MTBR-tau212-221. The proteolytic MTBR-tau243 peptides may comprise one or more of MTBR-tau243-256V and MTBR-tau243-256V (dN). The protease may comprise trypsin. The CSF sample may comprise an internal standard. The internal standard may be one or more of ¹⁵N-0N3R-tau and ¹⁵N-2N4R-tau. The method may further comprise one or more of detecting and quantifying one or more of amyloid beta, N-terminal tau, mid-domain tau, and post-translational modifications of tau, and may comprise identifying an ApoE isoform in the biological or CSF sample. Quantifying amyloid beta step may comprise quantifying one or more of an Aβ42/40 value and post-translational modifications of tau, which may comprise one or more of phospho-tau217, phospho-tau205, phospho-tau181, phospho-tau153, phospho-tau111, and phospho-tau208.

Provided herein is a method of detecting insoluble tau aggregates in a plasma sample, which may comprise (a) performing affinity depletion on a plasma sample by contacting the sample with affinity depletion agents comprising one or more epitope-binding agents that each binds to one of N-terminal tau, mid-domain tau, and long-MTBR tau, but not to an antigen within amino acids 235-256 of SEQ ID NO: 1, wherein the plasma sample comprises cleaved fragments of tau, to obtain a depleted sample and an enriched sample, wherein the depleted sample comprises N-terminal tau, mid-domain tau, and long-MTBR tau, and wherein the enriched sample is enriched for endogenously cleaved fragments of tau comprising amino acids 235-254 of SEQ ID NO: 1 (endogenous MTBR-tau243 peptides); (b) performing immunoprecipitation on the enriched sample by contacting the enriched sample with an immunoprecipitation agent comprising an epitope-binding agent that binds to endogenous MTBR-tau243 peptides, to obtain a purified sample; (c) contacting the endogenous MTBR-tau243 peptides with a protease comprising an Arg-C endopeptidase to obtain a sample comprising proteolytic MTBR-tau243 peptides; and (d) detecting the proteolytic MTBR-tau243 peptides by performing liquid chromatography-mass spectrometry (LC/MS) or an immunoassay, wherein detecting one or more of MTBR-tau243-256V, MTBR-tau243-256V (dN) and MTBR-tau212-221 is indicative of insoluble tau aggregates in the plasma sample. The MTBR-tau243 peptides in step (d) may comprise one or more of MTBR-tau243-256V, MTBR-tau243-256V (dN) and MTBR-tau212-221. The sample comprising proteolytic MTBR-tau243 peptides may be desalted before step (d). The affinity depletion agents may comprise an epitope-binding agent that specifically binds to an epitope within amino acids 1 to 243 of SEQ ID NO: 1; a first epitope-binding agent that specifically binds to an epitope within amino acids 1 to 103 of tau-441, and a second epitope-binding agent that specifically binds to an epitope within amino acids 104 to 243 of SEQ ID NO: 1; or, the first epitope-binding agent, the second epitope-binding agent, and a third epitope-binding agent that specifically binds to an epitope within amino acids 257-264 of SEQ ID NO: 1. The first epitope-binding agent may be HJ8.5, the second epitope-binding agent may be one or more of Tau1 and HJ8.7, and the third epitope-binding agent may be HJ34.8. The affinity depletion agents may comprise Tau1, HJ8.5, and HJ8.7. The affinity depletion agents may further comprise HJ34.8. The immunoprecipitation agent may comprise HJ32.11. The plasma sample may comprise an internal standard. The internal standard may comprise ¹⁵N-2N4R-tau. The purified sample may comprise an internal standard. The internal standard may comprise one or more of ¹³N¹⁵N-2N4R-tau and ¹⁵N-0N3R-tau. The method may further comprise one or more of detecting and quantifying one or more of amyloid beta, N-terminal tau, mid-domain tau, and post-translational modifications of tau, and may comprise identifying an ApoE isoform in the biological or CSF sample. Quantifying amyloid beta step may include quantifying one or more of an Aβ42/40 value and post-translational modifications of tau, which may comprise one or more of phospho-tau217, phospho-tau205, phospho-tau181, phospho-tau153, phospho-tau111, and phospho-tau208.

Provided herein is a method of detecting an Alzheimer disease (AD)-related pathology in a subject, which may comprise detecting insoluble tau aggregates in a CSF or plasma sample according to a method described above, wherein the CSF or plasma sample is from the subject, and wherein detecting insoluble aggregates of tau is indicative of the AD-related pathology in the subject. The AD-related pathology may be tau deposition in the subject's brain. The AD-related pathology may be amyloid beta deposition in the subject's brain or brain arteries.

Provided herein is a method of detecting Alzheimer disease (AD)-related tau deposition in a brain of a subject, which may comprise detecting insoluble tau aggregates in a CSF or plasma sample according to a method described above, wherein the CSF or plasma sample is from the subject, and wherein detecting insoluble aggregates of tau is indicative of AD-related tau deposition in the brain of the subject.

Provided herein is a method diagnosing Alzheimer's disease in a subject, which may comprise (a) detecting insoluble tau aggregates in a CSF or plasma sample according to any one of the method described above, wherein the CSF or plasma sample is from the subject; and (b) diagnosing Alzheimer's disease when the amount of a proteolytic MTBR-tau243 peptide detected differs by about 1.5σ or more above the mean of a healthy control population, wherein σ is the standard deviation defined by the normal distribution measured in a control population that is amyloid negative as measured by PET imaging and/or Aβ42/40 measurement in CSF.

Provided herein is a method of measuring Alzheimer disease (AD) progression in a subject, which may comprise (a) detecting insoluble tau aggregates in a first and a second CSF or plasma sample according to any one of the methods described above; and (b) calculating a difference between amounts of a proteolytic MTBR-tau243 peptide in the second sample and the first sample, wherein a statistically significant increase in the amount of the proteolytic MTBR-tau243 peptide in the second sample as compared to the first sample indicates progression of the subject's Alzheimer's disease.

The subject of any of the methods described above may have the follow characteristics. The subject may be amyloid negative or amyloid positive. The subject may have no dementia. The subject may have dementia. The subject may have a clinical dementia rating (CDR) score of 0.5 to 1.0. The subject may have a CDR score of >1.0 to 2.0 (moderate AD). The subject may have a CDR score of >2.0.

The methods may further comprise quantifying amyloid beta, quantifying N-terminal tau, quantifying mid-domain tau, quantifying post-translational modifications of tau, or identifying an ApoE isoform in the biological or CSF sample. The quantifying amyloid beta step may include quantifying an Aβ42/40 value, and quantifying post-translational modifications of tau include quantifying one or more of phospho-tau217, phospho-tau205, phospho-tau181, phospho-tau153, phosphor-tau111, and phospho-tau208.

Provided herein is a method of treating a tauopathy in a subject in need thereof, which may comprise (a) detecting insoluble tau aggregates in a CSF or plasma sample according to any one of the methods described above, wherein the CSF or plasma sample is from the subject; and (b) administering to the subject a treatment that may alter tau pathology, wherein the amount of a proteolytic MTBR-tau243 peptide detected may differ by about 1.56 or more above the mean of a healthy control population, wherein σ is the standard deviation defined by the normal distribution measured in a control population that does not have clinical signs or symptoms of a tauopathy and may be amyloid negative as measured by PET imaging and/or Aβ42/40 measurement in CSF, and wherein the amount of the proteolytic MTBR-tau243 peptide may be indicative of a tauopathy.

Provided herein is a method of treating a tauopathy in a subject in need thereof, which comprises administering to the subject a treatment that alters tau pathology, wherein the subject has been identified as having an amount of a proteolytic MTBR-tau243 peptide detected according to any one of the methods described above, wherein the amount differs by about 1.5σ or more above the mean of a healthy control population, wherein σ is the standard deviation defined by the normal distribution measured in a control population that does not have clinical signs or symptoms of a tauopathy and may be amyloid negative as measured by PET imaging and/or Aβ42/40 measurement in CSF, and wherein the amount of the proteolytic MTBR-tau243 peptide may be indicative of a tauopathy. The treatment may alter or may stabilize the amount of the detected proteolytic MTBR-tau243 peptide. The treatment may be selected from the group consisting of lecanemab, donanemab, AADvac1, ACI-3024, ACI-35, APNmAb005, ASN51, AZP2006, BIIB076, BIIB080, BIIB113, Bepranemab, Dasatinib+Quercetin, E2814, Epothilone D, Gosuranemab, JNJ-63733657, LM™, LY3372689, Lu AF87908, MK-2214, NIO752, OLX-07010, PNT001, PRX005, RG7345, Rember™, Semorinemab, TPI 287, Tideglusib, Tilavonemab, Zagotenemab, an anti-tau monoclonal antibody, an anti-tau anti-sense oligonucleotide, an anti-tau small interfering RNA, an tau production inhibitor, and a tau active vaccine. The treatment may be selected from the group consisting of anti-Aβ antibodies, anti-tau antibodies, anti-TREM2 antibodies, TREM2 agonists, gamma-secretase inhibitors, beta-secretase inhibitors, a kinase inhibitor, a phosphatase activator, a vaccine, and a tau protein aggregation inhibitor. The kinase inhibitor may be an inhibitor of a thousand-and-one amino acid kinase (TAOK), CDK, GSK-3β, MARK, CDK5, or Fyn. The phosphatase activator may increase the activity of protein phosphatase 2A. The vaccine may be CAD106 or AF20513. The anti-Aβ antibody may be aducanumab or another anti-amyloid antibody that may remove plaques.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIGS. 1A-B show associations between CSF biomarkers and amyloid- and tau-PET. Associations between CSF biomarkers and amyloid-PET (FIG. 1A) and tau-PET (FIG. 1B). First two columns show scatter plots of MTBR-tau243 (first column) and pT217/T217 (second column) and amyloid-(n=268) or tau-PET (n=443) in BioFINDER-2 participants, color-coded by diagnosis and amyloid status. Linear regression models, adjusting for age and sex, were used to obtain β, p-values (asterisks) and R2 shown in the plots. Scatter plots for all the biomarkers in both cohorts are shown in FIGS. 6 and 7A-B. The third and fourth columns show standardized β of the association between each CSF biomarker and amyloid- or tau-PET in BioFINDER-2 and Knight ADRC participants (n=219; except for pT231/T231 in which n=184 for all cases), respectively. Solid and dashed lines show standardized β (central dot) and 95% CI when all participants or only amyloid positive participants (BioFINDER-2: amyloid-PET: n=172, tau-PET: n=287; Knight ADRC: n=136; except for pT231/T231 in which n=117) were included, respectively. Asterisks (crosses) show the highest or not significantly different standardized β in all (amyloid positive only) participants, in each cohort and outcome based on bootstrapping. Thus, those biomarkers without asterisks or crosses have statistically lower correlations. Aβ-positive participants were selected based on CSF Aβ42/40 previously validated cut-offs (CSF Aβ42/40<0.08 in BioFINDER-2 and CSF Aβ42/40<0.0673 in Knight ADRC). Association p-values were derived from two-sided tests, and bootstrapping p-values were obtained from one-sided tests, all without adjustment for multiple comparisons. All p-values from associations between CSF biomarkers and amyloid- and tau-PET were <0.001. Abbreviations: Aβ+, amyloid positive; AD+, Alzheimer's disease dementia amyloid positive; CSF, cerebrospinal fluid; CU−, cognitively unimpaired amyloid negative; CU+, cognitively unimpaired amyloid positive; MCI+, mild cognitive impairment amyloid positive; MTBR, microtubule binding region; non-AD, non-Alzheimer's disease dementia; PET, positron emission tomography; SUVR, standardized uptake value ratio.

FIGS. 2A-B show proportion of variation of CSF biomarker levels explained by amyloid- and tau-PET The partial R² values are displayed within the columns, while the percentages of partial R ²over the total R² of the model are indicated above each column. These values were computed using individual CSF biomarkers as outcomes and amyloid and tau measures as predictors in separate linear regression models adjusted for age and sex, within each CSF biomarker and cohort. The percentages may not sum up to 100% due to potential shared variance. The biomarkers are arranged from left to right based on the increasing contribution (%) of tau to their levels.

Abbreviations: CSF, cerebrospinal fluid; MTBR, microtubule binding region; PET, positron emission tomography.

FIGS. 3A-B show longitudinal CSF biomarkers change by baseline amyloid and tau status Rates of change in CSF biomarkers per baseline amyloid (A) and tau (T) status are depicted (pT231/T231: n=218, rest: n=220). Individual rates of change are represented by dots. Trajectories for each group are displayed as boxplots, which were generated using linear mixed models (the central band represents the median, the lower and upper hinges correspond to the first and third quartiles, and the whiskers depict the maximum/minimum value or 1.5 times the interquartile range from the hinge, whichever is lower). Differences among all groups were assessed using Kruskal-Wallis tests, and pairwise Wilcoxon tests were employed for post-hoc comparisons. Asterisks indicate the p-values from two-sided tests without correction for multiple comparisons. Longitudinal CSF data was available only in BioFINDER-2. Amyloid-positive participants were identified using previously validated cut-offs for CSF Aβ42/40 (CSF Aβ42/40<0.08). Tau positivity was determined based on tau-PET SUVR in the meta-ROI (SUVR>1.32). The actual p-values for A−T− vs. A+T− were: p=0.011 (pT205/T205); for A−T− vs. A+T+ were: p=0.011 (pT181/T181), p=0.014 (pT205/T205), p=0.617 (pT217/T217), and p=0.980 (pT231/T231); and for A+T− vs. A+T+ were: p=0.788 (pT205/T205), p=0.007 (pT231/T231). All other comparisons yielded p<0.001.*, p<0.050; **, p<0.010; ***, p<0.001. Abbreviations: A−T−, amyloid and tau negative; A+T−, amyloid positive and tau negative; A+T+, amyloid positive, tau positive; CSF, cerebrospinal fluid; MTBR, microtubule binding region; PET, positron emission tomography; SUVR, standardized uptake value ratio.

FIGS. 4A-B show associations between CSF biomarkers and MMSE Associations between CSF biomarkers and MMSE are depicted for BioFINDER-2 (FIG. 4A, n=342) and Knight ADRC (FIG. 4B, pT231/T231: n=184, rest: n=219) participants. The first two columns display scatter plots of MTBR-tau243 (first column) and pT217/T217 (second column) against MMSE, color-coded by diagnosis and amyloid status. In the BioFINDER-2 cohort, orange dots represent MCI+ participants, while in the Knight ADRC cohort, they represent individuals with very mild AD. Linear regression models, adjusted for age and sex, were utilized to obtain β coefficients, p-values, and R² values shown in the plots. Scatter plots for all biomarkers in both cohorts can be found in FIGS. 9A-B. The third column shows the standardized β coefficients for all biomarkers, along with the associations of amyloid-PET and CSF Aβ42/40 (reversed) and tau-PET for comparison. Solid and dashed lines represent the standardized β coefficients (central dot) and 95% confidence intervals (95% CI) when including all participants or only amyloid-positive participants (BioFINDER-2: n=261, Knight ADRC: n=136, except for pT231/T231, where n=117), respectively. Asterisks (crosses) indicate the highest or not significantly different standardized β coefficients in all (amyloid-positive only) participants within each cohort and outcome, based on bootstrapping. Non-AD participants from BioFINDER-2 were excluded from these analyses. Amyloid-positive participants were selected using previously validated cut-offs for CSF Aβ42/40 (CSF Aβ42/40<0.08 in BioFINDER-2 and CSF Aβ42/40<0.0673 in Knight ADRC). Association p-values were derived from two-sided tests, and bootstrapping p-values were obtained from one-sided tests, all without adjustment for multiple comparisons. All p-values for associations between CSF biomarkers and MMSE were <0.001. Abbreviations: Aβ, amyloid-β; Aβ+, amyloid positive; AD+, Alzheimer's disease dementia amyloid positive; CSF, cerebrospinal fluid; CU−, cognitively unimpaired amyloid negative; CU+, cognitively unimpaired amyloid positive; MCI+, mild cognitive impairment amyloid positive; MMSE, Mini-Mental State Examination; MTBR, microtubule binding region; PET, positron emission tomography; SUVR, standardized uptake value ratio.

FIGS. 5A-B show predicting quantitative amyloid-PET, tau-PET, and MMSE continuous measures with CSF biomarkers. Linear regression models were employed to predict amyloid-PET (first column, BioFINDER-2: n=256), tau-PET (second column, BioFINDER-2: n=422), and cognition (MMSE, third column, BioFINDER-2: n=342) in BioFINDER-2 (FIG. 5A) and Knight ADRC (FIG. 5B, n=184). The base model included age and sex (and education for MMSE) as predictors. The parsimonious model was derived by using LASSO regression to identify the optimal combination of CSF biomarkers and demographic factors (age, sex, and/or education). Biomarkers included in the parsimonious models are indicated by a black border, and their names are shown in bold. The other models solely employed individual CSF biomarkers as predictors. For comparison, CSF Aβ42/40 and tau-PET were used as predictors in independent models for predicting all outcomes and cognition only, respectively. Model comparisons were conducted using an F-test for nested models or Vuong's test for non-nested models. Non-AD cases were excluded from the BioFINDER-2 cohort for the cognition analyses. Abbreviations: Aβ, amyloid-β; CSF, cerebrospinal fluid; CU−, cognitively unimpaired amyloid negative; MMSE, Mini-Mental State Examination; LASSO, least absolute shrinkage and selection operator; MTBR, microtubule binding region; PET, positron emission tomography.

FIG. 6 shows CSF biomarkers by diagnosis. Levels of each CSF biomarkers by clinical diagnosis and amyloid status in the BioFINDER-2 cohort (n=448). Amyloid-positive participants were selected based on CSF Aβ42/40 (CSF Aβ42/40<0.0 8). Larger pink dots represent two MAPT R40 6W mutation carriers (one CU− and the other in FTD groups), both amyloid-negative but with substantial tau-PET binding. In boxplots, central band represents the median of the group, the lower and upper hinges correspond to the first and third quartiles, and the whiskers represent the maximum/minimum value or the 1.5 IQR from the hinge, whatever is lower). Differences in CSF biomarker levels by diagnostic groups were tested using ANCOVA adjusted for age and sex. Post hoc analyses were performed two-sided using the Tuckey test. Actual p values are reported in Table 3 for space reasons. p<0.0 50; **, p<0.0 10;***, p<0 .0 0 1. Abbreviations: AD+, Alzheimer's disease dementia amyloid positive; CBS, corticobasal syndrome; CU−, cognitively unimpaired amyloid negative; CU+, cognitively unimpaired amyloid positive; FTD, frontotemporal dementia; MCI+, mild cognitive impairment amyloid positive; MTBR, microtubule-binding region; PD, Parkinson's disease; PDD, Parkinson's disease dementia; PPA, primary progressive aphasia; PSP, progressive supranuclear palsy.

FIGS. 7A-B show associations between all CSF biomarkers and Aβ-PET. Associations between CSF biomarkers and amyloid-PET (Centiloid) in BioFINDER-2 (FIG. 7A) and Knight ADRC (FIG. 7B) participants. Linear regression models, adjusting for age and sex, were used to obtain standardized β and p values shown in the plots. In the BioFINDER-2 cohort, orange dots represent MCI+ participants whereas in the Kinght-ADRC cohort, represent very mild AD. *, p<0.050;**, p<0.010; ***, p<0.001. Abbreviations: AD+, Alzheimer's disease dementia amyloid positive; CL, Centiloids, CSF, cerebrospinal fluid; CU−, cognitively unimpaired amyloid negative; CU+, cognitively unimpaired amyloid positive; MCI+, mild cognitive impairment amyloid positive; MTBR, microtubule binding region; non-AD, non-Alzheimer's disease dementia; PET, positron emission tomography.

FIGS. 8A-B show associations between all CSF biomarkers and tau-PET in the Braak I-IV ROI. Associations between all CSF biomarkers and tau-PET in the Braak I-IV ROI. Associations between CSF biomarkers and tau-PET in the Braak I-IV ROI (SUVR) in BioFINDER-2 (FIG. 8A) and Knight ADRC (FIG. 8B) participants. Linear regression models, adjusting for age and sex, were used to obtain standardized β and p-values shown in the plots. In the BioFINDER-2 cohort, orange dots represent MCI+ participants whereas in the Kinght-ADRC cohort, represent very mild AD. *, p<0.050; **, p<0.010; ***, p<0.001. Abbreviations: AD+, Alzheimer's disease dementia amyloid positive; CSF, cerebrospinal fluid; CU−, cognitively unimpaired amyloid negative; CU+, cognitively unimpaired amyloid positive; MCI+, mild cognitive impairment amyloid positive; MTBR, microtubule binding region; non-AD, non-Alzheimer's disease dementia; PET, positron emission tomography, SUVR, standardized uptake value ratio.

FIGS. 9A-C show associations between CSF biomarkers and tau-PET in different Braak regions. Associations between CSF biomarkers and tau-PET in Braak I (FIG. 9A), Braak III-IV (FIG. 9B) and Braak V-VI (FIG. 9C) regions. First two columns show scatter-plots of MTBR-tau243 (first column) and pT217/T217 (second column) and tau-PET in BioFINDER-2 participants (n=443), colored by diagnosis and amyloid status. Linear regression models, adjusting for age and sex, were used to obtain standardized β, p-values (asterisks) and R2 shown in the plots. Third and fourth columns show standardized β of the association between each CSF biomarker and tau-PET in BioFINDER-2 and Knight ADRC participants (n=219; except for pT231/T231 in which n=184), respectively. Solid and dashed lines show standardized β (central dot) and 95% CI when all participants or only amyloid positive participants (BioFINDER-2: n=287; Knight ADRC, n=136) were included, respectively. Asterisks (crosses) show the highest or not significantly different standardized β in all (amyloid positive only) participants, in each cohort and outcome. Amyloid-positive participants were selected based on CSF Aβ42/40 previously validated cutoffs (CSF Aβ42/40<0.0 8 in BioFINDER-2 and CSF Aβ42/40<0.0 673 in Knight ADRC). Association p-values were based on two-sided tests and bootstrapping p-values from one-sided tests, all unadjusted for multiple comparisons. All p-values from associations between CSF biomarkers and tau-PET shown in the scatter-plots were <0.001. *, p<0.050; **, p<0.010;***, p<0.001. Abbreviations: Aβ+, amyloid positive; AD+, Alzheimer's disease dementia amyloid positive; CSF, cerebrospinal fluid; CU−, cognitively unimpaired amyloid negative; CU+, cognitively unimpaired amyloid positive; MCI+, mild cognitive impairment amyloid positive; MTBR, microtubule binding region; non-AD, non-Alzheimer's disease dementia; PET, positron emission tomography; SUVR, standardized uptake value ratio.

FIG. 10 shows longitudinal CSF biomarkers trajectories by baseline “A” and “T” status. Individual (shaded lines) and group (bold lines) CSF biomarker levels trajectories over time based on their A/T baseline status. Statistical differences to the reference group (A−T−) are shown with asterisks with the appropriate color. Longitudinal CSF data was only available in BioFINDER-2. Amyloid-positive participants were selected based on a CSF Aβ42/40 previously validated cutoff (CSF Aβ42/40<0.0 8). Tau positivity was assessed based on tau-PET SUVR in the meta-ROI (Braak I-IV SUVR>1.32). *, p<0.0 50; **, p<0.0 10; ***, p<0.00 1. Abbreviations: A−T−, amyloid and tau negative; A+T−, amyloid positive and tau negative; A+T+, amyloid positive, tau positive; CSF, cerebrospinal fluid; MTBR, microtubule binding region; PET, positron emission tomography.

FIGS. 11A-B show associations between all CSF biomarkers and MMSE. In BioFINDER-2 cohort (FIG. 11A), orange dots represent MCI+ participants whereas in the Kinght-ADRC cohort (FIG. 11B), represent very mild AD. Linear regression models, adjusting for age, sex and years of education were used to obtain standardized β and p-values shown in the plots. *, p<0.50; **, p<0.010; ***, p<0.001. Abbreviations: AD+, Alzheimer's disease dementia amyloid positive; CSF, cerebrospinal fluid; CU−, cognitively unimpaired amyloid negative; CU+, cognitively unimpaired amyloid positive; MCI+, mild cognitive impairment amyloid positive; MMSE, Mini Mental State Examination; MTBR, microtubule binding region; PET, positron emission tomography, SD, standard deviation; SUVR, standardized uptake value ratio.

FIGS. 12A-B show associations between all CSF biomarkers and CSF Aβ42/40. Associations between CSF biomarkers and CSF Aβ42/40 in BioFINDER-2 (FIG. 12A, n=427) and Knight ADRC (FIG. 12B, n=219; except for pT231/T231 in which n=184) participants. Linear regression models, adjusting for age and sex, were used to obtain standardized β, p-values (asterisks) and R2 are shown in the plots. P-values were based on two-sided tests unadjusted for multiple comparisons. *, p<0.050; **, p<0.010; ***, p<0.001. Abbreviations: AD+, Alzheimer's disease dementia amyloid positive; CL, Centiloids, CSF, cerebrospinal fluid; CU−, cognitively unimpaired amyloid negative; CU+, cognitively unimpaired amyloid positive; MCI+, mild cognitive impairment amyloid positive; MTBR, microtubule binding region; non-AD, non-Alzheimer's disease dementia; PET, positron emission tomography.

FIGS. 13A-B show proportion of variation of CSF biomarker levels explained by CSF Aβ42/40 and tau-PET. Proportion of variation of CSF biomarker levels explained by CSF Aβ42/40 and tau-PET is shown as partial R² for each biomarker in BioFINDER-2 (FIG. 13A, n=422) and Knight ADRC (FIG. 13B, n=184). Partial R² values are shown inside columns. Percentages of partial R²over the total R² of the model are shown on top of each column. Partial R² and total R²were computed using each CSF biomarker as outcome and amyloid and tau measures as predictors in a linear regression model adjusted for age and sex, in independent models for each CSF biomarker and cohort. Percentages do not add to 100% as possible shared variance. Biomarkers are ordered (from left to right) by the increasing contribution (%) of tau on their levels. Abbreviations: CSF, cerebrospinal fluid; CU−, cognitively unimpaired amyloid negative; MTBR, microtubule binding region; PET, positron emission tomography.

FIG. 14 shows longitudinal CSF biomarkers change by baseline amyloid and tau status. CSF biomarker rates of change per baseline amyloid (A), as measured by PET, and tau (T), as measured by PET, status (n=174). Dots represent individual rates of change. Boxplots represent trajectories per group as assessed by linear mixed models (central band of the boxplot represents the median of the group, the lower and upper hinges correspond to the first and third quartiles, and the whiskers represent the maximum/minimum value or the 1.5 IQR from the hinge, -whatever is lower). Differences among all groups were tested using a Kruskal-Wallis tests and pairwise Wilcoxon tests for post-hoc comparisons. P-values come from two-sided tests uncorrected for multiple comparisons and are represented by asterisks. Longitudinal CSF data was only available in BioFINDER-2. Amyloid-positive participants were selected based on amyloid-PET previously validated cut-offs (SUVR>1.03). Tau positivity was assessed based on tau-PET SUVR in the meta-ROI (SUVR>1.32). Actual p-values for A−T− vs. A+T− were: p=0.024 (pT205/T205); for A−T− vs. A+T+ were: p=0.277 (pT181/T181), p=0.041 (pT205/T205), p=0.178 (pT217/T217) and p=0.368 (pT231/T231); and for A+T− vs. A+T+ were: p=0.001 (pT181/T181), p=0.738 (pT205/T205), p=0.011 (pT217/T217), p=0.023 (pT231/T231). The rest of comparisons rendered a p<0.001.*, p<0.050; **, p<0.010; ***, p<0.001. Abbreviations: A−T−, amyloid and tau negative; A+T−, amyloid positive and tau negative; A+T+, amyloid positive, tau positive; CSF, cerebrospinal fluid; MTBR, microtubule binding region; PET, positron emission tomography; SUVR, standardized uptake value ratio.

FIGS. 15A-B show predicting AD-related continuous measure by CSF biomarkers in amyloid-positive participants. Linear regression models were used for predicting amyloid-PET (first column, n=172), tau-PET (second column, n=287) and MMSE (third column, n=261) in BioFINDER-2 (FIG. 15A) and Knight ADRC (FIG. 15B, n=117) amyloid positive participants. Base model included age and sex (and education for MMSE) as predictors. Parsimonious model was obtained with the optimal combination of CSF biomarkers and demographics (age and/or sex and/or education) assessed using a LASSO regression. Biomarkers included in the parsimonious models are depicted a black border and their name is shown in bold. The other models used only individual CSF biomarkers as predictors. CSF Aβ42/40 and tau-PET were used as predictor in independent models for predicting cognition as a comparison. Models were compared using an F-test (nested models) or Vuong's test (non-nested models). Non-AD cases were excluded from BioFINDER-2 cohort for the cognition analyses. Amyloid-positive participants were selected based on CSF Aβ42/40 previously validated cut-offs (CSF Aβ42/40<0.08 in BioFINDER-2 and CSF Aβ42/40<0.0673 in Knight ADRC). Abbreviations: Aβ, amyloid-β; CSF, cerebrospinal fluid; CU−, cognitively unimpaired amyloid negative; MMSE, Mini-Mental State Examination; LASSO, least absolute shrinkage and selection operator; MTBR, microtubule binding region; PET, positron emission tomography.

FIGS. 16A-B show a method of CSF tau analysis. (FIG. 16A) Schematic of binding sites of antibodies used in this study and (FIG. 16B) the sample preparation scheme. CSF tau species were immunoprecipitated by Tau1 and HJ series (clone name: HJ8.5, HJ8.7, HJ32.11, and HJ34.8) antibodies. For all antibodies, 3 mg/gram sepharose beads were generated. For Tau1 immunoprecipitation (IP), 1.1 ug antibody/sample was used. For HJ8.5 and HJ8.7 IP, 2.3 μg antibody/sample was used. For HJ32.11 and HJ34.8 IP, 11.25 μg antibody/sample was used. CSF (450 μL) was mixed with 20 μL of solution containing 100 ug/μL each of 15N 0N3R-tau and 15N 2N4R-tau as internal standards. The tau species consisting primarily of N-terminal to mid-domain regions and late MTBR region (around the residue 260 in R1) were immunoprecipitated with Tau1, HJ8.5, HJ8.7, and HJ34.8 antibodies. Subsequently, 20 μL of 13C15N 2N4R-tau internal standard (100 pg/μL) was spiked into the post-immunoprecipitated CSF. Then, the MTBR-tau243 species containing the residue 243 was immunoprecipitated with the other novel antibody HJ32.11 in the post-immunoprecipitated CSF. All immunoprecipitated tau species (p-tau and MTBR-tau243) were digested by trypsin and desaltated by C18 toptip (Glygen). The resulting tau peptides were analyzed by nanoAcquity ultra-performance LC system (Waters) coupled to Orbitrap Tribrid Eclipse mass spectrometer (Thermo Scientific) operating in parallel reaction monitoring mode. MS transitions were extracted using Skyline v.22.2.2.278 (MacCoss lab, University of Washington). Data were aggregated using Tableau v.2022.2.2 (Tableau Software) to calculate CSF biomarker levels.

FIG. 17 is a schematic of the longest human tau isoform (2N4R). The N-terminus (N term), mid domain, MTBR, and C-terminus (C term) are identified for this isoform and will vary in a predictable way for other tau isoforms (e.g. 2N3R, 1NR4, 1N3R, 0N4R, and 0N3R).

FIGS. 18A-B show exemplary sequential immunodepletion/immunoprecipitation protocols to isolate fragments of MTBR-tau243 from CSF. (FIG. 18A) Method 1 (design no. 1) uses Tau1/HJ8.5/HJ8.7 in the first step and HJ32.11 in the second step. (FIG. 18B) Method 2 (design no. 2) uses Tau1/HJ8.5/HJ8.7 and HJ34.8 in the first step followed by HJ32.11 in the second step. FIG. 18C shows the affinity of antibodies to MTBR-tau243 fragments.

FIG. 19 shows short MTBR-tau243 species are diverse and only short fragment is associated with AD tau tangles in biofluid. The figure also shows the regions of tau to which HJ8.5, HJ8.7, Tau1, E2814, 77G7, and HJ34.8 antibodies bind. The short MTBR-tau243 and MTBR-tau212-221 fragments are captured by HJ32.11 antibody.

FIGS. 20A-B show the concentration of fragments precipitated by the two methods in FIG. 18. FIG. 20A shows HJ34.8 antibody precipitates a sub-set of MTBR-tau species in CSF. FIG. 20B shows the depletion effect of MTBR-tau243 by HJ34.8 antibody in the second immunoprecipitation and/or immunodepletion step using HJ32.11 antibody.

FIGS. 21A-B show the correlation between the concentration of MTBR-tau243 detected by chemical extraction (CX) and each of the two immunoprecipitation and/or immunodepletion methods in FIG. 18. (FIG. 21A) Correlation of MTBR-tau243 using immunoprecipitation and/or immunodepletion using Tau1/HJ8.5/HJ8.7. (FIG. 21B) Correlation of MTBR-tau243 using immunoprecipitation and/or immunodepletion using Tau1/HJ8.5/HJ8.7/HJ34.8. Tau species precipitated by HJ34.8 do not improve the correlation with the CSF MTBR-tau243 prepared by the chemical extraction method.

FIGS. 22A-B show the correlation between the concentration of MTBR-tau243 detected by Tau PET and each of the two immunoprecipitation and/or immunodepletion methods previously described. (FIG. 22A) Correlation of MTBR-tau243 using immunoprecipitation and/or immunodepletion using Tau1/HJ8.5/HJ8.7. (FIG. 22B) Correlation of MTBR-tau243 using immunoprecipitation and/or immunodepletion using Tau1/HJ8.5/HJ8.7/HJ34.8. The tau species precipitated by HJ34.8 is not correlated with tau-PET, so the depletion at the first step is conducted. Eventually, the second immunoprecipitation using HJ32.11 shows better correlation with tau-PET.

FIGS. 23A-D show MTBR-tau243 is the most associated with Tau-PET even in amyloid (+) only population, but not with Amyloid-PET (contrast characteristic with pT217). pT205 is the intermediate characteristic (between MTBR243 and pT217) to recapitulate both amyloid and tau pathology.

FIGS. 24A-C show MTBR-tau243 maintains the discrimination power to identify tau pathology in Amyloid-positive sub-group (B).

FIG. 25 shows an exemplary sequential immunodepletion or immunoprecipitation protocol to detect short fragments of MTBR-tau243 in plasma.

FIGS. 26A-B show plasma MTBR-tau243 development is challenging compared to CSF.

FIG. 27 is a scheme to identify the endogenous MTBR-tau243 species which surrogates AD tau pathology.

FIG. 28 shows MTBR-tau243 sequence quantified in CSF protocol using trypsin digestion. The MTBR-tau243-254 have multiple lysine (K) residues around MTBR-tau243. The tau sequence shown (SRLQTAPVPMPDLKNVKSKIGSTENLKHQP; SEQ ID NO: 22) is amino acids 241-270 of tau-441.

FIG. 29 shows a modified one-step immunoprecipitation method using HJ32.11 beads and ArgC digestion in CSF for detecting MTBR-tau243.

FIGS. 30A-B show the drastic increase in the amounts of MTBR-tau243-256V (SEQ ID NO: 15) and 243-256V @255N-deamidation (SEQ ID NO: 16) fragment detected in samples with high Tau-Pet levels.

FIGS. 31A-B show the correlation of of MTBR-tau243-255 (SEQ ID NO: 17) (FIG. 31A) and MTBR-tau 243-255 with @255N-deamidation (SEQ ID NO: 10) (FIG. 31B) to Tau-PET levels in CSF. The MTBR-tau243-255 with @255N-deamidation species showed a better specificity to low, middle, and high Tau-PET levels compared to the non-deamidated fragment.

FIGS. 32A-B show the correlation of of MTBR-tau243-257 (SEQ ID NO: 18) (FIG. 32A) and MTBR-tau 243-257 with @255N-deamidation (SEQ ID NO: 11) (FIG. 32B) to Tau-PET in CSF. The MTBR-tau243-257 with @255N-deamidation species showed a better specificity to low, middle, and high Tau-PET levels compared to the non-deamidated fragment.

FIGS. 33A-B show the correlation of MTBR-tau243-252D (SEQ ID NO: 13) (FIG. 33A) and MTBR-tau 243-258S with @255N-deamidation (SEQ ID NO: 14) (FIG. 33B) to Tau-PET in CSF. The MTBR-tau243-258S with @255N-deamidation species showed a better specificity to low, middle, and high Tau-PET levels compared to the non-deamidated fragment.

FIGS. 34A-B show the correlation of MTBR-tau243-253L (SEQ ID NO: 12) (FIG. 34A) and MTBR-tau 243-254K with @244-deamidation (SEQ ID NO: 14) (FIG. 34B) to Tau-PET in CSF. Both species plateau from middle to high Tau-PET.

FIGS. 35A-B show MTBR-tau243-254 (SEQ ID NO: 21) and MTBR-tau243-256V (SEQ ID NO: 15) detected in plasma samples. The amount of MTBR-tau243-256V detected increases dramatically in samples with high Tau-PET levels.

FIGS. 36A-B show MTBR-tau243-254 (SEQ ID NO: 21), MTBR-tau243-256V (SEQ ID NO: 15), and MTBR-tau243-256V @255N-deamidation (SEQ ID NO: 16) detected in plasma samples.

FIGS. 37A-F show LC chromatographs and mass spectrum of MTBR-tau243-256V @255N-deamidation (SEQ ID NO: 16).

FIG. 38 shows an exemplary method of detecting MTBR-tau243-256 (SEQ ID NO: 21) and MTBR-tau243-256V-dN (SEQ ID NO: 15) in plasma.

FIG. 39 shows an exemplary method of detecting MTBR-tau243-256 (SEQ ID NO: 21) and MTBR-tau243-256V-dN (SEQ ID NO: 15) in plasma.

FIGS. 40A-C show how the results of detecting MTBR-tau243 in CSF translates to plasma (FIG. 40A), and a comparison of MTBR-tau243 detection in plasma vs. tau PET in plasma (FIG. 40B) and CSF (FIG. 40C).

FIG. 41 shows how the 243-256V @255N-deamidation (SEQ ID NO: 16) fragment provides meaningful information about the presence of tangles based on the use of HJ32.11 and HJ34.8 in a method of detection in plasma as compared to CSF.

FIG. 42 shows a possible model explaining why plasma 243-256V @255N-deamidation (SEQ ID NO: 16) in plasma is very specific to mature tangles, as compared to detecting MTBR-tau243 in CSF.

FIGS. 43A-C show CSF pT217/T217, CSF MTBR-tau243, and plasma MTBR-tau243-256dN for Alzheimer's disease staging (n=55). Plasma MTBR-tau243-256dN was notably increased after AD onset (CDR≥1).

FIG. 44 shows that MTBR-tau212 is highly associated with MTBR-tau243 (Rho>0.9), in samples from a Knight Alzheimer's Disease Research Center cohort, suggesting that MTBR-tau212-221 is equivalent to Tau-PET as a marker.

FIG. 45 shows that MTBR-tau212 is highly associated with MTBR-tau243 (Rho>0.9) in samples from a Biofinder2 cohort, suggesting that MTBR-tau212-221 is equivalent to Tau-PET as a marker.

### DETAILED DESCRIPTION

The inventors have discovered that fragments of tau containing MTBR-tau243 (that is, fragments containing amino acid of tau-441 as disclosed herein) can be detected in either cerebrospinal fluid (CSF) or plasma, and developed methods for performing the detection. They have also discovered that, surprisingly, MTBR-tau243 is a specific marker for insoluble tau aggregates (e.g., neurofibrillary tangles) associated with tauopathies, including Alzheimer's disease (AD). MTBR-tau243 fragments disclosed herein may be used as an alternative to tau-positron emission tomograpahy (PET) assays. Thus, the inventors have discovered a simpler, less expensive approach to diagnosing tauopathies.

The inventors have also surprisingly discovered that the MTBR-tau243 fragments disclosed herein can be used to determine whether a patient's symptoms are due to AD. About 20-30% of patients who are at least 65 years old have amyloid plaques, but no symptoms. Detecting amyloid plaques using available markers such as Aβ42, pTau-217, pTau-181, and pTau-231 can lead to a false positive diagnosis of AD, because the presence of amyloid plaques is not specific to AD symptoms. Detecting the presence of MTBR-tau243 fragments as disclosed herein indicates that a patient's symptoms are due to AD, and not just associated with it.

### 1. Definitions

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

Discussed below are components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules of the compound are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Other aspects and iterations of the invention are described more thoroughly below.

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “a” or “an” entity refers to one or more of that entity; for example, a “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone).

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

By “specifically binds,” it is meant that a binding molecule, e.g., an antibody or antigen-binding fragment thereof binds to an epitope via its antigen binding domain, and that the binding entails some recognition between the antigen binding domain and the epitope. According to this definition, a binding molecule is said to “specifically bind” to an epitope when it binds to that epitope, via its antigen-binding domain more readily than it would bind to a random, unrelated epitope. Thus, the term “specifically binds,” as used herein with regards to epitope binding agents, means that an epitope binding agent does not cross react to a significant extent with other epitopes on the protein of interest (e.g., Tau), or on other proteins in general.

The term “antibody”, as used herein, is used in the broadest sense and encompasses various antibody and antibody-like structures, including but not limited to full-length monoclonal, polyclonal, and multispecific (e.g., bispecific, trispecific, etc.) antibodies, as well as heavy chain antibodies and antibody fragments provided they exhibit the desired antigen-binding activity. The domain(s) of an antibody that is involved in binding an antigen is referred to as a “variable region” or “variable domain,” and is described in further detail below. A single variable domain may be sufficient to confer antigen-binding specificity. Preferably, but not necessarily, antibodies useful in the discovery are produced recombinantly. Antibodies may or may not be glycosylated, though glycosylated antibodies may be preferred. An “isolated” antibody is one which has been separated from a component of its natural environment. In some embodiments, an antibody is purified to greater than 95% or 99% purity as determined by methods known in the art.

In addition to antibodies described herein, it may be possible to design an antibody mimetic or an aptamer using methods known in the art that functions substantially the same as an antibody of the invention. An “antibody mimetic” refers to a polypeptide or a protein that can specifically bind to an antigen but is not structurally related to an antibody. Antibody mimetics have a mass of about 3 kDa to about 20 kDa. Non-limiting examples of antibody mimetics are affibody molecules, affilins, affimers, alphabodies, anticalins, avimers, DARPins, and monobodies. Aptamers are a class of small nucleic acid ligands that are composed of RNA or single-stranded DNA oligonucleotides and have high specificity and affinity for their targets. Aptamers interact with and bind to their targets through structural recognition, a process similar to that of an antigen-antibody reaction. Aptamers have a lower molecular weight than antibodies, typically about 8-25 kDa. As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art. See, for instance U.S. Pat. No. 7,939,313, herein incorporated by reference in its entirety.

The terms “full length antibody” and “intact antibody” may be used interchangeably, and refer to an antibody having a structure substantially similar to a native antibody structure or having heavy chains that contain an Fc region as defined herein. The basic structural unit of a native antibody comprises a tetramer. Each tetramer is composed of two identical pairs of polypeptide chains, each pair having one “light” chain (about 25 kDa) and one “heavy” chain (about 50-70 kDa). Light chains are classified as gamma, mu, alpha, and lambda. Heavy chains are classified as gamma, mu, alpha, delta, or epsilon, and define the antibody's isotype as IgG, IgM, IgA, IgD and IgE, respectively. The amino-terminal portion of each light and heavy chain includes a variable region of about 100 to 110 or more amino acid sequences primarily responsible for antigen recognition (VL and VH, respectively). The carboxy-terminal portion of each chain defines a constant region primarily responsible for effector function. Within light and heavy chains, the variable and constant regions are joined by a “J” region of about 12 or more amino acid sequences, with the heavy chain also including a “D” region of about 10 more amino acid sequences. Intact antibodies are properly cross-linked via disulfide bonds, as is known in the art.

The variable domains of the heavy chain and light chain of an antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, anti-bodies that bind a particular antigen may be isolated using a VH or VL domain from an antibody that binds the antigen to screen a library of complementary VL or VH do-mains, respectively. See, e.g., Portolano et al., J. Immunol. 150:880-887 (1993); Clarkson et al., Nature 352:624-628 (1991).

“Framework region” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence: FR1-HVR1-FR2-HVR2-FR3-HVR3-FR4. The FR domains of a heavy chain and a light chain may differ, as is known in the art.

The term “hypervariable region” or “HVR” as used herein refers to each of the regions of a variable domain which are hypervariable in sequence (also commonly referred to as “complementarity determining regions” or “CDR”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). As used herein, “an HVR derived from a variable region” refers to an HVR that has no more than two amino acid substitutions, as compared to the corresponding HVR from the original variable region. Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); (d) CDR1-IMGT (positions 27-38), CDR2-IMGT (positions 56-65), and CDR3-IMGT regions (positions 105-116 or 105-117), which are based on IMGT unique numbering (Lefranc, “The IMGT unique numbering for Immunoglobulins, T cell receptors and Ig-like domains,” The Immunologist, 1999, 7:132-136; Lefranc et al., Nucleic Acids Research, 2009, 37 (Database issue): D1006-D1012; Ehrenmann et al., “Chapter 2: Standardized Sequence and Structure Analysis of Antibody Using IMGT,” in Antibody Engineering Volume 2, Eds. Roland E. Kontermann and Stefan Dubel, 2010, Springer-Verlag Berlin Heidelberg, doi: 10.1007/978-3-642-01147-4; imgt.org/IMGTScientificChart/Nomenclature/IMGT-FRCDRdefinition), and (e) combinations of (a), (b), (c), and/or (d), as defined below for various antibodies of this disclosure. Unless otherwise indicated, HVR residues and other residues in the variable domain (e.g., FR residues) that are assigned sequence identification numbers are numbered based on IMGT unique numbering, supra.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “variant Fc region” comprises an amino acid sequence that can differ from that of a native Fc region by virtue of one or more amino acid substitution(s) and/or by virtue of a modified glycosylation pattern, as compared to a native Fc region or to the Fc region of a parent polypeptide. In an example, a variant Fc region can have from about one to about ten amino acid substitutions, or from about one to about five amino acid substitutions in a native sequence Fc region or in the Fc region of the parent polypeptide. The variant Fc region herein may possess at least about 80% homology, at least about 90% homology, or at least about 95% homology with a native sequence Fc region and/or with an Fc region of a parent polypeptide.

An “antibody fragment” refers to a molecule other than an intact antibody that comprises a portion of an intact antibody that binds the antigen to which the intact antibody binds. Non-limiting examples of antibody fragments include but are not limited to Fv, Fab, Fab′, Fab′-SH, F(ab′)2; single-chain forms of antibodies and higher or-der variants thereof; single-domain antibodies, and multi-specific antibodies formed from antibody fragments.

Single-chain forms of antibodies, and their higher order forms, may include, but are not limited to, single-domain antibodies, single chain variant fragments (scFvs), divalent scFvs (di-scFvs), trivalent scFvs (tri-scFvs), tetravalent scFvs (tetra-scFvs), diabodies, and triabodies and tetrabodies. ScFv's are comprised of heavy and light chain variable regions connected by a linker. In most instances, but not all, the linker may be a peptide. A linker peptide is preferably from about 5 to 30 amino acids in length, or from about 10 to 25 amino acids in length. Typically, the linker allows for stabilization of the variable domains without interfering with the proper folding and creation of an active binding site. In preferred embodiments, a linker peptide is rich in glycine, as well as serine or threonine. ScFvs can be used to facilitate phage display or can be used for flow cytometry, immunohistochemistry, or as targeting domains. Methods of making and using scFvs are known in the art. ScFvs may also be conjugated to a human constant domain (e.g. a heavy constant domain is derived from an IgG do-main, such as IgG1, IgG2, IgG3, or IgG4, or a heavy chain constant domain derived from IgA, IgM, or IgE). Diabodies, triabodies, and tetrabodies and higher order variants are typically created by varying the length of the linker peptide from zero to several amino acids. Alternatively, it is also well known in the art that multivalent binding antibody variants can be generated using self-assembling units linked to the variable domain.

A “single-domain antibody” refers to an antibody fragment consisting of a single, monomeric variable antibody domain.

Multi-specific antibodies include bi-specific antibodies, tri-specific, or anti-bodies of four or more specificities. Multi-specific antibodies may be created by combining the heavy and light chains of one antibody with the heavy and light chains of one or more other antibodies. These chains can be covalently linked.

An antibody of the disclosure may be a Dual-affinity Re-targeting Antibody (DART). The DART format is based on the diabody format that separates cognate variable domains of heavy and light chains of the 2 antigen binding specificities on 2 separate polypeptide chains. Whereas the 2 polypeptide chains associate noncovalently in the diabody format, the DART format provides additional stabilization through a C-terminal disulfide bridge. DARTs can be produced in high quantity and quality and reveal exceptional stability in both formulation buffer and human serum.

“Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

A “heavy chain antibody” refers to an antibody that consists of two heavy chains. A heavy chain antibody may be an IgG-like antibody from camels, llamas, alpacas, sharks, etc., or an IgNAR from a cartiliaginous fish.

A “humanized antibody” refers to a non-human antibody that has been modified to reduce the risk of the non-human antibody eliciting an immune response in humans following administration but retains similar binding specificity and affinity as the starting non-human antibody. A humanized antibody binds to the same or similar epitope as the non-human antibody. The term “humanized antibody” includes an antibody that is composed partially or fully of amino acid sequences derived from a human antibody germline by altering the sequence of an antibody having non-human hypervariable regions (“HVR”). The simplest such alteration may consist simply of substituting the constant region of a human antibody for the murine constant region, thus resulting in a human/murine chimera which may have sufficiently low immunogenicity to be acceptable for pharmaceutical use. Preferably, the variable region of the antibody is also humanized by techniques that are by now well known in the art. For example, the framework regions of a variable region can be substituted by the corresponding human framework regions, while retaining one, several, or all six non-human HVRs. Some framework residues can be substituted with corresponding residues from a non-human VL domain or VH domain (e.g., the non-human antibody from which the HVR residues are derived), e.g., to restore or improve specificity or affinity of the humanized antibody. Substantially human framework regions have at least about 75% homology with a known human framework sequence (i.e. at least about 75%, at least about 80%, at least about 85%, at least about 90%, at least about 95%, or at least about 99% sequence identity). HVRs may also be randomly mutated such that binding activity and affinity for the antigen is maintained or enhanced in the context of fully human germline framework regions or framework regions that are substantially human. As mentioned above, it is sufficient for use in the methods of this discovery to employ an antibody fragment. Further, as used herein, the term “humanized antibody” refers to an antibody comprising a substantially human framework region, at least one HVR from a nonhuman antibody, and in which any constant region present is substantially human. Substantially human constant regions have at least about 90% with a known human constant sequence (i.e. about 90%, about 95%, or about 99% sequence identity). Hence, all parts of a humanized antibody, except possibly the HVRs, are substantially identical to corresponding pairs of one or more germline human immunoglobulin sequences.

If desired, the design of humanized immunoglobulins may be carried out as follows or using similar methods familiar to those with skill in the art (for example, see Almagro, et al. Front. Biosci. 2008, 13 (5): 1619-33). A murine antibody variable region is aligned to the most similar human germline sequences (e.g. by using BLAST or similar algorithm). The CDR residues from the murine antibody sequence are grafted into the similar human “acceptor” germline. Subsequently, one or more positions near the CDRs or within the framework (e.g., Vernier positions) may be reverted to the original murine amino acid in order to achieve a humanized antibody with similar binding affinity to the original murine antibody. Typically, several versions of humanized antibodies with different re-version mutations are generated and empirically tested for activity. The humanized antibody variant with properties most similar to the parent murine antibody and the fewest murine framework reversions is selected as the final humanized antibody candidate.

The term “Aβ” refers to peptides derived from a region in the carboxy terminus of a larger protein called amyloid precursor protein (APP). The gene encoding APP is located on chromosome 21. There are many forms of Aβ that may have toxic effects: Aβ peptides are typically 37-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically. For example, two such Aβ isoforms include Aβ40 and Aβ42; with the Aβ42 isoform being particularly fibrillogenic or insoluble and associated with disease states. The term “Aβ” typically refers to a plurality of Aβ species without discrimination among individual Aβ species. Specific Aβ species are identified by the size of the peptide, e.g., Aβ42, Aβ40, Aβ38 etc.

As used herein, the term “Aβ42/Aβ40 value” means the ratio of the amount of Aβ42 in a sample obtained from a subject compared to the amount of Aβ40 in the same sample.

“Aβ amyloidosis” is defined as clinically abnormal Aβ deposition in the brain. A subject that is determined to have Aβ amyloidosis is referred to herein as “amyloid positive,” while a subject that is determined to not have Aβ amyloidosis is referred to herein as “amyloid negative.” There are accepted indicators of Aβ amyloidosis in the art. At the time of this disclosure, Aβ amyloidosis is directly measured by amyloid imaging (e.g., PiB PET, fluorbetapir, or other imaging methods known in the art) or indirectly measured by decreased cerebrospinal fluid (CSF) Aβ42 or a decreased CSF Aβ42/40 ratio. [11C]PIB-PET imaging with mean cortical binding potential (MCBP) score >0.18 is an indicator of Aβ amyloidosis, as is cerebral spinal fluid (CSF) Aβ42 concentration of about 1 ng/ml measured by immunoprecipitation and mass spectrometry (IP/MS)). Alternatively, a cut-off ratio for CSF Aβ42/40 that maximizes the accuracy in predicting amyloid-positivity as determined by PIB-PET can be used. Values such as these, or others known in the art and/or used in the examples, may be used alone or in combination to clinically confirm Aβ□amyloidosis. See, for example, Klunk W E et al. Ann Neurol 55 (3) 2004, Fagan A M et al. Ann Neurol, 2006, 59 (3), Patterson et. al, Annals of Neurology, 2015, 78 (3): 439-453, or Johnson et al., J. Nuc. Med., 2013, 54 (7): 1011-1013, each hereby incorporated by reference in its entirety. Subjects with Aβ amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer's Disease (AD), cerebral amyloid angiopathy (CAA), Lewy body dementia, and inclusion body myositis. Subjects with Aβ amyloidosis are at an increased risk of developing a disease associated with Aβ amyloidosis.

A “clinical sign of Aβ amyloidosis” refers to a measure of Aβ deposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk W E et al. Ann Neurol 55 (3) 2004, and Fagan A M et al. Ann Neurol 59 (3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ39, Aβ40, and/or total Aβ), as described in U.S. patents Ser. Nos. 14/366,831, 14/523,148 and 14/747,453, each hereby incorporated by reference in its entirety. Additional methods are described in Albert et al. Alzheimer's & Dementia 2007 Vol. 7, pp. 170-179; McKhann et al., Alzheimer's & Dementia 2007 Vol. 7, pp. 263-269; and Sperling et al. Alzheimer's & Dementia 2007 Vol. 7, pp. 280-292, each hereby incorporated by reference in its entirety. Importantly, a subject with clinical signs of Aβ amyloidosis may or may not have symptoms associated with Aβ deposition. Yet subjects with clinical signs of Aβ amyloidosis are at an increased risk of developing a disease associated with Aβ amyloidosis.

A “candidate for amyloid imaging” refers to a subject that has been identified by a clinician as an individual for whom amyloid imaging may be clinically warranted. As a non-limiting example, a candidate for amyloid imaging may be a subject with one or more clinical signs of Aβ amyloidosis, one or more Aβ plaque associated symptoms, one or more CAA associated symptoms, or combinations thereof. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

An “Aβ plaque associated symptom” or a “CAA associated symptom” refers to any symptom caused by or associated with the formation of amyloid plaques or CAA, respectively, being composed of regularly ordered fibrillar aggregates called amyloid fibrils. Exemplary Aβ plaque associated symptoms may include, but are not limited to, neuronal degeneration, impaired cognitive function, impaired memory, altered behavior, emotional dysregulation, seizures, impaired nervous system structure or function, and an increased risk of development or worsening of Alzheimer's disease or CAA. Neuronal degeneration may include a change in structure of a neuron (including molecular changes such as intracellular accumulation of toxic proteins, protein aggregates, etc. and macro level changes such as change in shape or length of axons or dendrites, change in myelin sheath composition, loss of myelin sheath, etc.), a change in function of a neuron, a loss of function of a neuron, death of a neuron, or any combination thereof. Impaired cognitive function may include but is not limited to difficulties with memory, attention, concentration, language, abstract thought, creativity, executive function, planning, and organization. Altered behavior may include, but is not limited to, physical or verbal aggression, impulsivity, decreased inhibition, apathy, decreased initiation, changes in personality, abuse of alcohol, tobacco or drugs, and other addiction-related behaviors. Emotional dysregulation may include, but is not limited to, depression, anxiety, mania, irritability, and emotional incontinence. Seizures may include but are not limited to generalized tonic-clonic seizures, complex partial seizures, and non-epileptic, psychogenic seizures. Impaired nervous system structure or function may include, but is not limited to, hydrocephalus, Parkinsonism, sleep disorders, psychosis, impairment of balance and coordination. This may include motor impairments such as monoparesis, hemiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “control population,” “normal population” or a sample from a “healthy” subject refers to a subject, or group of subjects, who are clinically determined to not have a tauopathy or Aβ amyloidosis, or a clinical disease associated with Aβ amyloidosis (including but not limited to Alzheimer's disease), based on qualitative or quantitative test results.

As used herein, the term “blood sample” refers to a biological sample derived from blood, preferably peripheral (or circulating) blood. The blood sample can be whole blood, plasma or serum, although plasma is typically preferred.

The term “isoform”, as used herein, refers to any of several different forms of the same protein variants, arising due to alternative splicing of mRNA encoding the protein, post-translational modification of the protein, proteolytic processing of the protein, genetic variations and somatic recombination. The terms “isoform” and “variant” are used interchangeably.

The term “tau” refers to a plurality of isoforms encoded by the gene MAPT (or homolog thereof), as well as species thereof that are C-terminally truncated in vivo, N-terminally truncated in vivo, post-translationally modified in vivo, or any combination thereof. As used herein, the terms “tau” and “tau protein” and “tau species” may be used interchangeably. In many animals, including but not limited to humans, non-human primates, rodents, fish, cattle, frogs, goats, and chicken, tau is encoded by the gene MAPT. In animals where the gene is not identified as MAPT, a homolog may be identified by methods well known in the art.

In humans, there are six isoforms of tau that are generated by alternative splicing of exons 2, 3, and 10 of MAPT. These isoforms range in length from 352 to 441 amino acids. Exons 2 and 3 encode 29-amino acid inserts each in the N-terminus (called N), and full-length human tau isoforms may have both inserts (2N), one insert (1N), or no inserts (0N). All full-length human tau isoforms also have three repeats of the microtubule binding domain (called R). Inclusion of exon 10 at the C-terminus leads to inclusion of a fourth microtubule binding domain encoded by exon 10. Hence, full-length human tau isoforms may be comprised of four repeats of the microtubule binding domain (exon 10 included: R1, R2, R3, and R4) or three repeats of the microtubule binding domain (exon 10 excluded: R1, R3, and R4). Human tau may or may not be post-translationally modified. For example, it is known in the art that tau may be phosphorylated, ubiquinated, glycosylated, and glycated. Human tau also may or may not be proteolytically processed in vivo at the C-terminus, at the N-terminus, or at the C-terminus and the N-terminus. Accordingly, the term “human tau” encompasses the 2N3R, 2N4R, IN3R, IN4R, 0N3R, and 0N4R isoforms, as well as species thereof that are C-terminally truncated in vivo, N-terminally truncated in vivo, post-translationally modified in vivo, or any combination thereof. Alternative splicing of the gene encoding tau similarly occurs in other animals.

The term “tau-441,” as used herein, refers to the longest human tau isoform (2N4R), which is 441 amino acids in length. The amino acid sequence of tau-441 is provided below:

(SEQ ID NO: 1)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRV
QSKIGSLDNITHVPGGGNKKIETHKLTFRENAKAKTDHGAEIVYKSPVVS
GDTSPRHLSNVSSTGSIDMVDSPQLATLADEVSASLAKQGL

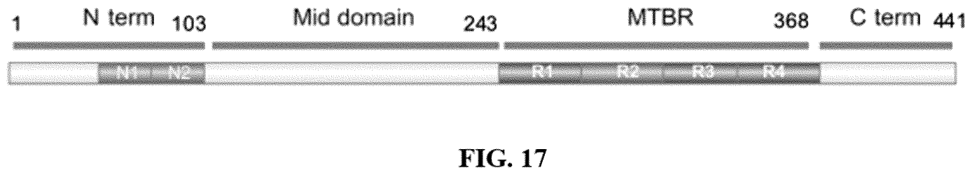

The N-terminus (N term), mid-domain, MTBR, and C-terminus (C term) are identified in FIG. 17 for this isoform. These regions will vary in a predictable way for other tau isoforms (e.g., 2N3R, 1 NR4, 1 N3R, 0N4R, and 0N3R). Accordingly, when amino acid positions are identified relative to tau-441, a skilled artisan will be able to determine the corresponding amino acid position for the other isoforms.

The term “N-terminal tau,” as used herein, refers to a tau peptide, or a plurality of tau peptides, that comprise(s) two or more amino acids, or all, of the N-terminus of tau (e.g., amino acids 1-103 of tau-441, etc.).

The term “mid-domain tau,” as used herein, refers to a tau peptide, or a plurality of tau peptides, that comprise(s) two or more amino acids, or all, of the mid-domain of tau (e.g., amino acids 104-243 of tau-441, etc.).

The term “MTBR tau,” as used herein, refers to a tau peptide, or a plurality of tau peptides, that comprise(s) two or more amino acids, or all, of the microtubule binding region (MTBR) of tau (e.g., amino acids 244-368 of tau-441, etc.).

The term “long-MTBR tau,” as used herein refers to a tau peptide, or a plurality of tau peptides, that comprise(s) two or more, or all, of amino acids 259-368 of tau-441.

The term “very short MTBR-tau243,” as used herein refers to a tau peptide, or a plurality of tau peptides, that comprise(s) two or more, or all, of amino acids 235-254 of tau-441.

The term “C-terminal tau,” as used herein, refers to a tau peptide, or a plurality of tau peptides, that comprise(s) two or more amino acids, or all, of the C-terminus of tau (e.g., amino acids 369-441 of tau-441, etc.).

An “endogenously cleaved fragment of tau” refers to a peptide fragment of a tau protein produced by an in vivo proteolytic cleavage event. A “proteolytic fragment of tau” refers to a peptide fragment of a tau protein produced by an in vitro proteolytic cleavage event. Cleaved fragments of tau peptides may be referred to herein by their amino acid numbering with reference to tau-441, where the first number indicates the fragment comprises the referred to amino acid and the second number indicates that last amino acid of the fragment. For instance, “243-255N-deamidation” or “243-255 (dN)” or “243-255N @255N deamidation” refer to a cleaved fragment of tau which includes amino acids 243-255, where amino acid 255N is the end of the fragment and is post-translationally modified by deamidation at 255N, (e.g., LQTAPVPMPDLK (dN) (SEQ ID NO: 10); also referred to as MTBR-tau243-255dN). Non-limiting examples of other cleaved fragments of tau peptides identified include 243-257K @255N-deamidation (SEQ ID NO: 11); 243-253L (SEQ ID NO: 12); 243-252D (SEQ ID NO: 13); 243-258S @255N-deamidation (SEQ ID NO: 14); 243-256V (SEQ ID NO: 15); 243-256V @255N-deamidation (SEQ ID NO: 16; also referred to as 243-256VdN, 243-256V (dN) and 243-dN-256V,); and 212-221 (SEQ ID NO: 17).

A disease associated with tau deposition in the brain is referred to herein as a “tauopathy”. The term “tau deposition” is inclusive of all forms pathological tau deposits including but not limited to neurofibrillary tangles, neuropil threads, and tau aggregates in dystrophic neurites. Tauopathies known in the art include, but are not limited to, progressive supranuclear palsy (PSP), dementia pugilistica, chronic traumatic encephalopathy, frontotemporal dementia and parkinsonism linked to chromosome 17, Lytico-Bodig disease, Parkinson-dementia complex of Guam, tangle-predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration (CBD), argyrophilic grain disease (AGD), Frontotemporal lobar degeneration (FTLD), Alzheimer's disease (AD), and frontotemporal dementia (FTD).

Tauopathies are classified by the predominance of tau isoforms found in the pathological tau deposits. Those tauopathies with tau deposits predominantly composed of tau with three MTBRs are referred to as “3R-tauopathies”. Pick's disease is a non-limiting example of a 3R-tauopathy. For clarification, pathological tau deposits of some 3R-tauopathies may be a mix of 3R and 4R tau isoforms with 3R isoforms predominant. Intracellular neurofibrillary tangles (i.e. tau deposits) in brains of subjects with Alzheimer's disease are generally thought to contain both approximately equal amounts of 3R and 4R isoforms. Those tauopathies with tau deposits predominantly composed of tau with four MTBRs are referred to as “4R-tauopathies”. PSP, CBD, and AGD are non-limiting examples of 4R-tauopathies, as are some forms of FTLD. Notably, pathological tau deposits in brains of some subjects with genetically confirmed FTLD cases, such as some V334M and R406W mutation carriers, show a mix of 3R and 4R isoforms.

A clinical sign of a tauopathy may be aggregates of tau in the brain, including but not limited to neurofibrillary tangles. Methods for detecting and quantifying tau aggregates in the brain are known in the art (e.g., tau PET using tau-specific ligands such as [18F]THK5317, [18F]THK5351, [18F]AV1451, [11C]PBB3, [18F]MK-6240, [18F]RO-948, [18F]P I-2620, [18F]GTP1, [18F]PM-PBB3, and [18F]JNJ64349311, [18F]JNJ-067), etc.).

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. Accordingly, a subject in need of treatment may or may not have any symptoms or clinical signs of disease.

The phrase “tau therapy” collectively refers to any imaging agent, therapeutic treatment, and/or a prophylactic or preventative measure contemplated for, or used with, subjects at risk of developing a tauopathy, or subjects clinically diagnosed as having a tauopathy. Non-limiting examples of imaging agents include functional imaging agents (e.g. fluorodeoxyglucose, etc.) and molecular imaging agents (e.g., Pittsburgh compound B, florbetaben, florbetapir, flutemetamol, radiolabeled tau-specific ligands, radionuclide-labeled antibodies, etc.). Non-limiting examples of therapeutic agents include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Ab antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, b-2 adrenergic receptor agonists, nicotinic acetylcholine receptor agonists, 5-HT2A inverse agonists, alpha-2c adrenergic receptor antagonists, 5-HT 1Aand 1 D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-COA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

### 2. Composition

a. Anti-MTBR Tau Antibodies

Provided herein is an anti-MTBR-tau antibody. The anti-MTBR tau antibodies disclosed herein may be described or specified in terms of the epitope(s) that they recognize or bind. The portion of a target polypeptide that specifically interacts with the antigen binding domain of an antibody is an “epitope.” Furthermore, it should be noted that an “epitope” on MTBR tau can be a linear epitope or a conformational epitope, and in both instances can include non-polypeptide elements, e.g., an epitope can include a carbohydrate or lipid side chain. The term “affinity” refers to a measure of the strength of the binding of an individual epitope with an antibody's antigen binding site. In one aspect, an antibody of the disclosure binds to a MTBR tau epitope located within amino acid amino acids 244-368 of tau-441 (SEQ ID NO: 1).

An “anti-MTBR tau antibody,” as used herein, refers to an isolated antibody that binds to human MTBR tau with an affinity constant or affinity of interaction (KD) between about 0.1 μM to about 10 μM, preferably about 0.1 μM to about 1 μM, more preferably about 0.1 μM to about 100 nM. Methods for determining the affinity of an antibody for an antigen are known in the art. Anti-MTBR tau antibodies useful herein include those which are suitable for administration to a subject in a therapeutic amount.

Anti-MTBR tau antibodies disclosed herein can also be described or specified in terms of their cross-reactivity. The term “cross-reactivity” refers to the ability of an antibody, specific for one antigen, to react with a second antigen; a measure of relatedness between two different antigenic substances. Thus, an antibody is cross-reactive if it binds to an epitope other than the one that induced its formation. The cross-reactive epitope generally contains many of the same complementary structural features as the inducing epitope, and in some cases, can actually fit better than the original. For example, certain antibodies have some degree of cross-reactivity, in that they bind related, but non-identical epitopes, e.g., epitopes with at least about 85%, at least about 90%, or at least about 95% identity (as calculated using methods known in the art) to a reference epitope. An antibody can be said to have little or no cross-reactivity if it does not bind epitopes with less than about 95%, less than about 90%, or less than about 85% identity to a reference epitope. An antibody can be deemed “highly specific” for a certain epitope, if it does not bind any other analog, ortholog, or homolog of that epitope.

The anti-MTBR tau antibody may comprise one or more of a heavy chain and a light chain variable region of HJ34.11. The anti-MTBR tau antibody may comprise one or more complementarity determining regions (CDR) 1-3 of one or more of the heavy and light chain variable regions of HJ34.11. The anti-MTBR tau antibody may comprise the heavy chain variable region CDR1-3 (H1-3) and the light chain variable region CDR1-3 (L1-3) of HJ34.11. In one example, the anti-MTBR tau antibody is HJ34.11 or an antigen binding fragment thereof. The anti-MTBR tau antibody may bind to an antigen contained within amino acids 226-264, and particularly amino acids 255-264, of tau relative to tau-441. The anti-MTBR tau antibody may bind to an endogenous peptide of tau containing amino acids 243-254 of tau relative to tau-441.

The sequences of the heavy and light chain variable regions of HJ34.11 are shown below. For heavy and light chain variable region sequences provided herein, it is known in the art that such sequences contain a signal peptide that may removed during post-translational processing of an antibody. Reference to a light or heavy chain variable region sequence encompasses a sequence lacking the respective signal peptide.

| | | SEQ |
|---|---|---|
| Antibody | | ID |
| portion | Sequence | NO: |
| HJ34.11 | MESQTQVFLSLLLWVSGTCGNIMITQSPSSLAVSAGEKVTMSCKSS | 2 |
| light | QSVLYSSDRKNYLAWYQQKPGQSPKLLIFWASTRESGVPDRFTGS | |
| chain | GSGTDFTLTINSVQAEDLAVYYCHQYLSSRTFGGGTKLEIK | |
| variable | | |
| region | | |
| HJ34.11 | MKCSWIIFFLMAVVTGVNSEVQLQQSGAELVKPGASVKLSCTASG | 3 |
| heavy | FNIKDYFMHWVKQRTEQGLEWIGRIDPEDDETKYAPKFQGKATIT | |
| chain | ADTSSNTVYLHLSSLTSEDTAVYYCARDGSTYSMDYWGQGTSVTV | |
| variable | SS | |
| region | | |

The anti-MTBR tau antibody may comprise one or more of a heavy chain and a light chain variable region of HJ32.8. The anti-MTBR tau antibody may comprise one or more complementarity determining regions (CDR) 1-3 of one or more of the heavy and light chain variable regions of HJ32.8. The anti-MTBR tau antibody may comprise the heavy chain variable region CDR1-3 and the light chain variable region CDR1-3 of HJ32.8. In one example, the anti-MTBR tau antibody is HJ32.8 or an antigen binding fragment thereof. The anti-MTBR tau antibody may bind to an antigen contained within amino acids 225-242, and particularly amino acids 225-235, of tau relative to tau-441. The anti-MTBR tau antibody may bind to an endogenous peptide of tau containing amino acids 243-254 of tau relative to tau-441.

The sequences of the heavy and light chain variable regions of HJ32.8 are shown below.

| | | SEQ |
|---|---|---|
| Antibody | | ID |
| portion | Sequence | NO: |
| HJ32.8 | MKLPVRLLVLMFWIPASSSDVLMTQTPLSLSVSLGDQVSISCRSSQS | 4 |
| light | IEHSNGNTYLEWYLQKPGQSPKLLIYKVSNRFSGVPDRFSGSGSGT | |
| chain | DFTLKISRVEAEDLGVYYCFQGLHVPYTFGGGTKLEIK | |
| variable | | |
| region | | |
| HJ32.8 | MKCSWIIFFLMAVVTGVNSEVQLQQSGAELVKPGASVKLSCTASG | 5 |
| heavy | FNIYDYFIHWVKQRTEQGLEWIGRIDPEDGEIKFAPKFQGKATITAD | |
| chain | TSSNTAYLQLSSLTSEDTAVYYCGCDFDYWGQGTTLTVSS | |
| variable | | |
| region | | |

The anti-MTBR tau antibody may comprise one or more of a heavy chain and a light chain variable region of HJ32.11. The anti-MTBR tau antibody may comprise one or more complementarity determining regions (CDR) 1-3 of one or more of the heavy and light chain variable regions of HJ32.11. The anti-MTBR tau antibody may comprise the heavy chain variable region CDR1-3 and the light chain variable region CDR1-3 of HJ32.11. In one example, the anti-MTBR tau antibody is HJ32.11 or an antigen binding fragment thereof. The anti-MTBR antibody may bind to an antigen contained within amino acids 225-242, and more particularly amino acids 235-242, of tau relative to tau-441. The anti-MTBR tau antibody may bind to an endogenous peptide of tau containing amino acids 243-254 of tau relative to tau-441. The anti-MTBR tau antibody may bind to an endogenous peptide of tau containing amino acids 243-256 of tau relative to tau-441, which may be MTBR-tau243-254, MTBR-tau243-256V or MTBR-tau243-256V @255N deamidation.

The sequences of the heavy and light chain variable regions of HJ32.11 are shown below.

| | | SEQ |
|---|---|---|
| Antibody | | ID |
| portion | Sequence | NO: |
| HJ32.11 | MKLPVRLLVLMFWIPASSSDVLMTQTPLSLPVSLGDQVSISCRSS | 6 |
| light | QSIVHSNGNTYLEWYLQKPGQSPKLLIYKVSNRFSGVPDRFSGSG | |
| chain | SGTDFTLKISRVEAEDLGVYYCFQGLHVPYTFGGGTKLEI | |
| variable | | |
| region | | |
| HJ32.11 | MKCSWIIFFLMAVVTGVNSEVQLQQSGAELVKPGASVRLSCTGS | 7 |
| heavy | GLNIKDYFIHWVKQRTKQGLEWIGRIDPEDDEIKYAPKFQGKATI | |
| chain | TADTSSNTAYLHLISLTSEDTAVYYCGWDFNYWGQGTTLTVSS | |
| variable | | |
| region | | |

The anti-MTBR tau antibody may comprise one or more of a heavy chain and a light chain variable region of HJ34.8. The anti-MTBR tau antibody may comprise one or more complementarity determining regions (CDR) 1-3 of one or more of the heavy and light chain variable regions of HJ34.8. The anti-MTBR tau antibody may comprise the heavy chain variable region CDR1-3 and the light chain variable region CDR1-3 of HJ34.8. In one example, the anti-MTBR tau antibody is HJ34.8 or an antigen binding fragment thereof. The anti-MTBR tau antibody may bind to an antigen contained within amino acids 226-264, and particularly amino acids 255-264, of tau relative to tau-441. The anti-MTBR tau antibody may bind to an endogenous peptide of tau containing amino acids 243-254 of tau relative to tau-441.

The sequences of the heavy and light chain variable regions of HJ34.8 are shown below.

| | | |
|---|---|---|
| HJ34.8 | MESQTQVFLSLLLWVSGTCGNIMITQSPSSLAVSAGEKVTMSCKS | 8 |
| light | SQSVLYSSDRKNYLAWYQQKPGQSPKLLIFWASTRESGVPDRFT | |
| chain | GSGSGTDFTLTINSVQAEDLAVYYCHQYLSSRTFGGGTKLEIK | |
| variable | | |
| region | | |
| HJ34.8 | MKCSWIIFFLMAVVTGVNSEVQLQQSGAELVKPGASVKLSCTAS | 9 |
| heavy | GFNIKDYFMHWVKQRTEQGLEWIGRIDPEDDETKYDPKFQGKAT | |
| chain | ITADTSFNTVYLHLSSLTSEDTAVYYCARDGSTYSMDYWGQGTS | |
| variable | VTVSS | |
| region | | |

In an exemplary embodiment, an anti-MTBR tau antibody according to the present disclosure comprises a VL that has one or more HVRs derived from SEQ ID NO: 2, SEQ ID NO: 4, SEQ ID NO: 6, or SEQ ID NO: 8; or a VH that has one or more HVRs derived from SEQ ID NO: 3, SEQ ID NO: 5, SEQ ID NO: 7, or SEQ ID NO: 9. The HVR derived from SEQ ID NO: 2, SEQ ID NO: 4, SEQ ID NO: 6, or SEQ ID NO: 8 may be L1, L2, L3, or any combination thereof. The HVR derived from SEQ ID NO: 3, SEQ ID NO: 5, SEQ ID NO: 7, or SEQ ID NO: 9 may be H1, H2, H3, or any combination thereof. The antibody comprising one or more HVRs derived from SEQ ID NO: 3, SEQ ID NO: 5, SEQ ID NO: 7, or SEQ ID NO: 9 may further comprise a light chain variable region (VL) comprising one or more HVRs derived from SEQ ID NO: 2, SEQ ID NO: 4, SEQ ID NO: 6, or SEQ ID NO: 8. The HVR may be L1, L2, L3, or any combination thereof. In various embodiments above, the antibody may be a humanized antibody, or the antibody may have a VL with 90, 91, 92, 93, 94, 95, 96, 97, 98, 99, or 100% identity to SEQ ID NO: 2, SEQ ID NO: 4, SEQ ID NO: 6, or SEQ ID NO: 8 and/or a VH with 90, 91, 92, 93, 94, 95, 96, 97, 98, 99 or 100% identity to SEQ ID NO: 3, SEQ ID NO: 5, SEQ ID NO: 7, or SEQ ID NO: 9. In each of the above embodiments, the anti-MTBR tau antibody may optionally comprise one or more constant regions, or a portion of a constant region, that is substantially human (i.e. at least 90%, 95%, or 99% sequence identity with a known human framework sequence). The present disclosure also encompasses the nucleic acid sequences encoding SEQ ID NO: 2, 3, 4, 5, 6, 7, 8, or 9, which can readily be determined by one of skill in the art, and may be incorporated into a vector or other large DNA molecule, such as a chromosome, in order to express an antibody of the disclosure.

In each of the above embodiments, L1, L2, L3, H1, H2, and H3 may be determined as
(a) amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996));
(d) CDR1-IMGT (positions 27-38), CDR2-IMGT (positions 56-65), and CDR3-IMGT regions (positions 105-116 or 105-117), which are based on IMGT unique numbering (Lefranc, “The IMGT unique numbering for Immunoglobulins, T cell receptors and Ig-like domains,” The Immunologist, 1999, 7:132-136; Lefranc et al., Nucleic Acids Research, 2009, 37 (Database issue): D1006-D1012; Ehrenmann et al., “Chapter 2: Standardized Sequence and Structure Analysis of Antibody Using IMGT,” in Antibody Engineering Volume 2, Eds. Roland E. Kontermann and Stefan Dubel, 2010, Springer-Verlag Berlin Heidelberg, doi: 10.1007/978-3-642-01147-4; imgt.org/IMGTScientificChart/Nomenclature/IMGT-FRCDRdefinition), and
(e) combinations of (a), (b), (c), and/or (d), as defined below for various antibodies of this disclosure.

In some embodiments, each of the exemplary antibodies described above may also contain a variant Fc region.

b. Components of the Composition

The present disclosure also provides pharmaceutical compositions encompassing an antibody described herein. Such compositions comprise an antibody as herein disclosed, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

### (1) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose de-rivatives, cellulose powder, cellulose esters (i.e., acetate and butyrate mixed esters), ethyl cellulose, methyl cellulose, hydroxypropyl cellulose, hydroxypropyl methylcell-lose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose mono-hydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

### (2) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

### (3) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

### (4) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

### (5) pH Modifier

In various embodiments, the excipient may be a pH modifier. By way of non-limiting example, the pH modifying agent may be sodium carbonate, sodium bicarbonate, sodium citrate, citric acid, or phosphoric acid.

### (6) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pre-gelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

### (7) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

### (8) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

### (9) Lubricant

In a further embodiment, the excipient may be a lubricant. Non-limiting examples of suitable lubricants include minerals such as talc or silica; and fats such as vegetable stearin, magnesium stearate, or stearic acid.

### (10) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

### (11) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

### (12) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

c. Administration Forms

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The present disclosure encompasses pharmaceutical compositions comprising an anti-MTBR tau antibody disclosed in the above, so as to facilitate administration and promote stability of the active agent. For example, an anti-MTBR tau antibody of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). Methods of preparing and administering anti-MTBR tau antibodies disclosed herein to a subject in need thereof are well known to or are readily determined by those skilled in the art. The route of administration of an anti-MTBR tau antibody can be, for example, peripheral, oral, parenteral, by inhalation or topical.

Pharmaceutical compositions for effective administration are deliberately designed to be appropriate for the selected mode of administration, and pharmaceutically acceptable excipients such as compatible carriers, dispersing agents, buffers, surfactants, preservatives, solubilizing agents, isotonicity agents, stabilizing agents and the like are used as appropriate.

Non-limiting examples of pharmaceutically acceptable carriers, include physiological saline, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, wool fat or a combination thereof.

Prevention of the action of microorganisms can be achieved by various antibacterial and antifungal agents, for example, parabens, chlorobutanol, phenol, ascorbic acid, thimerosal and the like. In many cases, isotonic agents can be included, for example, sugars, polyalcohols, such as mannitol, sorbitol, or sodium chloride in the composition.

Prolonged absorption of the injectable compositions can be brought about by including in the composition an agent which delays absorption, for example, aluminum monostearate and gelatin.

Compositions disclosed herein can be frozen or lyophilized for storage and reconstituted in a suitable sterile carrier prior to use.

In some embodiments, anti-MTBR tau antibodies may be formulated for parenteral administration. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions, as disclosed herein, can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an anti-MTBR tau antibody to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

As noted above, the agents or compositions described herein can also be used in combination with other therapeutic agents. Thus, in addition to the therapies described herein, one may also provide to the subject other therapies known to be efficacious for treatment of a disease, disorder, or condition.

The present disclosure encompasses pharmaceutical compositions comprising agents as disclosed above, so as to facilitate administration and promote stability of the active agent. For example, a compound of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and embodiments of the invention, the subject may be a human or any other animal.

### 3. Methods of Detecting Fragments of Tau

The present disclosure provides methods for detecting fragments of tau in a biological sample. The detecting may comprise confirming the presence of the fragments or quantifying the amount of the fragments. The fragments may be endogenously cleaved. In one example, the fragments of tau comprise one or more of a very short MTBR-tau243 or amino acids 212-254 of tau-441. The fragments of tau may be indicative of aggregated insoluble tau or tangles (e.g., neurofibrillary tangles) associated with a tauopathy. The detection and measuring may be performed by mass spectrometry or an immunoassay. The biological sample may be obtained from a subject having or suspected of having a tauopathy. The tauopathy may be AD.

The fragments of tau may comprise one or more of the peptides listed in Table 1, where the C-terminal amino acid represents the last amino acid of the C-terminus of the peptide.

TABLE 1
Cleaved fragments of tau

| NAME | SEQUENCE | SEQ ID NO: |
|---|---|---|
| 243-255N- | LQTAPVPMPDLK(dN) | 10 |
| deamidation | | |
| 243-257K @255N- | LQTAPVPMPDLK(dN)VK | 11 |
| deamidation | | |
| 243-253L | LQTAPVPMPDL | 12 |
| 243-252 | DLQTAPVPMPD | 13 |
| 243-258S @255N- | LQTAPVPMPDLK(dN)VKS | 14 |
| deamidation | | |
| 243-256V | LQTAPVPMPDLKNV | 15 |
| 243-256V @255N- | LQTAPVPMPDLK(dN)V | 16 |
| deamidation | | |
| 212-221 | TPSLPTPPTR | 17 |
| 243-257K | LQTAPVPMPDLKNVK | 18 |
| 243-255N | LQTAPVPMPDLKN | 19 |
| 243-254 @244 | LQTAPVPMPDLK | 20 |
| deamidation | | |
| 243-254 | LQTAPVPMPDLK | 21 |

In one example, the fragments of tau may comprise one or more of MTBR-tau243-256V, MTBR-tau243-256V (dN), and MTBR-tau212-221.

Detecting endogenously cleaved fragments of tau in a biological sample may comprise providing a biological sample and purifying cleaved fragments of tau from the biological sample. The cleaved fragment of tau may be performed without first exposing the cleaved fragment of tau to additional in vitro proteolytic cleavage. The cleaved fragment of tau may not be a tryptic tau peptide. A pre-clearing step (e.g., protein precipitation and/or immunodepleting) before or after purifying the cleaved fragment of tau may not be performed. Cleaved fragments of tau may be further cleaved in vitro with a protease before or after purification. The protease may comprise one or more of trypsin, a Lys-N endopeptidase, a Lys-C endopeptidase, an Asp-C endopeptidase, an Arg-N endopeptidase, and an Arg-C endopeptidase. In examples of detecting tau fragments in plasma, the protease comprises an Arg-C endopeptidase.

A sample of cleaved fragments of tau may be subject to one step or at least two steps of immunodepletion or immunoprecipitation. Each round of immunodepletion or immunoprecipitation may be performed using one or more anti-tau or anti-MTBR tau antibodies disclosed herein, or a combination thereof. After each imunodepletion or immunoprecipitation step, the purified tau fragments from one or more of the immunoprecipitated sample or the immunodepleted sample may be further cleaved with one or more proteases disclosed herein.

Liquid chromatography-mass spectrometry (LC/MS) or an immunoassay with a sample comprising a purified cleaved fragment of tau may be used to detect and measure the concentration (relative or absolute) of at least one cleaved fragment of tau. One or more cleaved fragments of tau may be used to detect and measure the amount of tau, such as the amount of insoluble tau aggregates, present in the biological sample. The immunoassay may comprise contacting a sample comprising a purified cleaved fragment of tau with an anti-MTBR tau antibody disclosed herein. In one example, the anti-MTBR tau antibody is capable of binding to MTBR-tau43 and comprises at least one of a light chain variable region and a heavy chain variable region of HJ32.11 or sequences thereof disclosed herein (for example, H1-H3 and L1-L3 of HJ32.11). In another example the anti-MTBR tau antibody is HJ32.11. In a further example, the anti-tau antibody competitively inhibits HJ32.11 binding. In one example, the immunoassay is an ELISA.

a. Detecting Tau in a Biological Sample

The method may comprise one or more of the following steps: (a) providing a biological sample selected from a blood sample or a CSF sample that comprises a cleaved fragment of tau, which may be endogenously cleaved; (b) purifying the cleaved fragment of tau from the biological sample without first exposing the cleaved fragment of tau to additional in vitro proteolytic cleavage; and (c) detecting the concentration of at least one purified cleaved fragment of tau. As used herein, detecting may comprise one or more of confirming the presence of, measuring, and quantifying. A blood sample disclosed herein may be a plasma sample.

In another example, the method comprises one or more of: (a) providing a biological sample selected from a blood sample or a CSF sample that comprises an endogeously cleaved fragment of tau; (b) purifying the cleaved fragment of tau from the biological sample, wherein the cleaved fragment of tau is optionally further cleaved in vitro with one or more proteases before or after purification; and (c) detecting the concentration of at least one purified cleaved fragment of tau.

The method may comprise one or more of: (a) providing a biological sample selected from a blood sample or a CSF sample; (b) purifying a cleaved fragment of tau from the biological sample; (c) optionally cleaving the purified cleaved fragment of tau with one or more proteases and then optionally desalting the resultant cleavage product by solid phase extraction to obtain a sample comprising proteolytic peptides of tau; and (d) performing liquid chromatography-mass spectrometry with the sample comprising proteolytic peptides of tau to detect and measure the amount of at least one proteolytic peptide of the cleaved fragment of tau.

In another example, a method of the present disclosure comprises decreasing in a biological sample by affinity depletion or immunoprecipitation of at least one peptide of tau comprising one or more of N-terminal tau, mid-domain tau, and long-MTBR tau. In one example, the peptides comprise N-terminal tau, mid-domain tau, and long-MTBR tau. Peptides of tau comprising N-terminal tau may be contacted with an antibody that binds to an antigen within N-terminal tau, which may be HJ8.5, HJ8.7, or both. Peptides of tau comprising mid-domain tau may be contacted with an antibody that binds to an antigen within mid-domain tau, which may be Tau1. Peptides of tau comprising the MTBR of tau may be contacted with an antibody that binds to an antigen within the MTBR, which may be HJ34.8. Peptides of tau comprising very short MTBR-tau243 may be contacted by an antibody that binds to an antigen within very short MTBR-tau243, which may be HJ32.11. The antibodies disclosed herein may be attached to a solid substrate, which may comprise a plate, a bead, or a column. In one example, the antibodies are attached to beads for use in immunodepletion or immunoprecipitation. For use in immunodepletion, immunoprecipitation, or immunodetection, the antibodies may be attached to a plate or a bead.

A biological sample comprising endogenously cleaved fragments of tau is contacted with one or more, or all of, HJ8.5, HJ.8.7, and Tau1. In one example, the biological sample is contacted with Tau1, HJ8.5, and HJ8.7. In another, the biological sample is contacted with Tau1, HJ8.5, HJ8.7, and HJ34.8. A sample depleted of tau peptides using the aforementioned antibodies may be proteolytically cleaved in vitro using one or more proteases disclosed herein, and the resulting in vitro cleaved tau fragments (proteolytic fragments) may be contacted with an antibody that binds to very short MTBR-tau243, such as HJ32.11. In one example, the proteases comprise an Arg-C endopeptidase.

The biological sample may be a CSF sample or a plasma sample. The biological sample may further comprise an isotope-labeled, tau internal standard. The method may comprise enriching a cleaved fragment of tau by a method that comprises purifying one or more cleaved fragments of tau from the affinity depleted sample. The enriched cleaved fragments of tau may be enriched for a very short MTBR-tau243 peptide. The very short MTBR-tau243 peptide may be detected and/or measured using an immunoassay, which may comprise use of HJ32.11. The sample comprising enriched very short MTBR-tau243 may be proteolytically cleaved with a protease. The resultant cleavage may be desalted by solid phase extraction to obtain a sample comprising peptides of tau. The peptides of tau may be detected by performing liquid chromatography-mass spectrometry (LC/MS) or an immunoassay of the sample comprising peptides of tau to detect and measuring the amount of at least one peptide of the cleaved fragment of tau.

While not required, in each of the above methods, removing additional proteins from the biological sample by protein precipitation and separation of the precipitated proteins to obtain a supernatant can be performed before or after purification of the cleaved fragment of tau.

### (1) Detecting Tau Peptides in CSF

Provided herein is a method of detecting a tau peptide in a CSF sample. The tau peptide may be a very short MTBR-tau243 peptide. The method may comprise contacting a CSF sample from a subject with anti-tau antibodies. In one example, an internal standard is added to the CSF sample. The internal standard may be one or both of ¹⁵N 0N3R-tau and ¹⁵N 2N4R-tau. The anti-tau antibodies may comprise one or more or all of HJ8.5, HJ8.7, and Tau1. The anti-tau antibodies may also comprise HJ34.8. Antibodies that competitively inhibit the aforementioned antibodies (e.g., N-terminal tau, mid-domain tau, and long-MTBR tau) may be used in the methods disclosed herein. As discussed herein, the antibodies may be attached to a solid substrate, which may be a bead. The sample may then be subjected to affinity depletion (immunodepletion) or immunoprecipitation, to form a first immunoprecipitate comprising immunoprecipitated tau peptides. An immunodepleted sample may be enriched for one or more of very short MTBR-tau243, MTBR-tau243-254, MTBR-tau243-256V, MTBR-tau243-256V (dN), and MTBR-tau212-221.

The immunoprecipitated tau peptides may be subjected to in vitro proteolytic cleavage by contacting tau peptides in the sample with a protease disclosed herein, which may comprise trypsin, and may further comprise a Lys-C endopeptidase. In one example, the protease does not comprise an Arg-C endopeptidase. In the resulting sample, a tau protein, which may be indicative of amyloid plaques, such is p-tau, may be detected. The p-tau may be one or more of p-tau181, p-tau205, p-tau217, and p-tau231.

The immunodepleted sample may be contacted with an antibody capable of binding to very short MTBR-tau43, which may be HJ32.11. In one example, an internal standard is added to the immunodepleted sample. The internal standard may be ¹³C¹⁵N 2N4R-tau. The immunodepleted sample may then be subjected to immunoprecipitation to form a second immunoprecipitate. Very short MTBR-tau243 in the second immunoprecipitate may be detected and/or measured in an immunoassay comprising contacting the very short MTBR-tau243 with an anti-tau antibody, which may be HJ32.11. The second immunoprecipitate may also be subjected to in vitro proteolytic cleavage by contacting tau peptides in the second immunoprecipitate with one or more proteases disclosed herein, which may comprise trypsin. Proteolytic peptides in the second immunoprecipitate may comprise one or more of MTBR-tau243-254, MTBR-tau243-256V, MTBR-tau243-256V (dN), and MTBR-tau212-221, which may be detected and/or measured using LC/MS. Before performing LC/MS, the second immunoprecipitate may be desalted.

b. Detecting Tau Peptides in Plasma

Provided herein is a method of detecting a tau peptide in a plasma sample. The tau peptide may be one or more of very short MTBR-tau243, MTBR-tau243-254, MTBR-tau243-256V, MTBR-tau243-256V (dN), and MTBR-tau212-221. In one example the peptide is MTBR-tau243-256V (dN). The method may comprise contacting a plasma sample from a subject with an anti-tau antibody. In one example, an internal standard is added to the CSF sample. The internal standard may be ¹⁵N 2N4R-tau. The anti-tau antibody may comprise one or more or all of HJ8.5, HJ8.7, and Tau1. The anti-tau antibody may also comprise HJ34.8. The sample may then be subjected to affinity depletion or immunoprecipitation, to form a first immunoprecipitate comprising immunoprecipitated tau peptides, and an immunodepleted sample which may be enriched for very short MTBR-tau243.

The immunoprecipitated tau peptides may be subjected to in vitro proteolytic cleavage by contacting tau peptides in the sample with a protease disclosed herein, which may comprise trypsin. In one example, the protease does not comprise an Arg-C endopeptidase. In the resulting sample, a tau protein, which may be indicative of amyloid plaques, such is p-tau, may be detected. The p-tau may be one or more of p-tau181, p-tau217, and p-tau231.

The immunodepleted sample may be contacted with an antibody capable of binding to very short MTBR-tau43, which may be HJ32.11. In one example, an internal standard is added to the immunodepleted sample. The internal standard may be one or more of ¹³N¹⁵N 2N4R-tau and ¹⁵N 0N3R-tau. The immunodepleted sample may then be subjected to immunoprecipitation to form a second immunoprecipitate. Very short MTBR-tau243 in the second immunoprecipitate may be detected and/or measured in an immunoassay comprising contacting the very short MTBR-tau243 with an anti-tau antibody, which may be HJ32.11. The second immunoprecipitate may also be subjected to in vitro proteolytic cleavage by contacting tau peptides in the second immunoprecipitate with one or more proteases comprising an Arg-C endopeptidase. The proteases may further comprise other proteases disclosed herein. Tau peptides in the second immunoprecipitate may comprise one or more of MTBR-tau243-254, MTBR-tau243-256V, MTBR-tau243-256 (dN), and MTBR-tau212-221, each of which may be detected and/or measured using LC/MS. In particular, one or more of MTBR-tau243-256V (dN) and MTBR-tau212-221 may be detected and/or measured. Prior to performing LC/MS, the second immunoprecipitate may be desalted.

In another example, the plasma sample is contacted with HJ32.11. The plasma sample may be supplemented with an internal reference disclosed herein. The contacted sample may be subjected to immunoprecipitation. Very short MTBR-tau243 in the resulting immunoprecipitate may be detected and/or measured in an immunoassay comprising contacting the immunoprecipitate with an anti-tau antibody capable of binding to very short MTBR-tau243, such as HJ32.11. The resulting immunoprecipitate sample may also be subjected to in vitro proteolytic cleavage with one or more proteolytic enzymes comprising an Arg-C endopeptidase. Proteolytically cleaved tau peptides, which may comprise one or more of MTBR-tau243-256, MTBR-tau243-256 (dN), and MTBR-tau212-221, may be detected and/or measured using LC/MS. In particular, one or more of MTBR-tau243-256 (dN) and MTBR-tau212-221 may be detected and/or measured. Prior to performing LC/MS, the second immunoprecipitate may be desalted.

c. Detecting Tau Peptides Other than Very Short MTBR-Tau243

The present disclosure further contemplates in each of the above methods determining the presence/absence of one or more proteins in the biological sample and/or measuring the concentration of one or more additional protein in the biological sample. In some embodiments, the one or more protein may be a protein depleted from the biological sample prior to purification of tau. For instance, in certain embodiments, N-terminal tau and/or mid-domain tau species may be identified and/or quantified separately from tau species (e.g., MTBR tau, C-terminal tau) quantified by the methods disclosed herein. In some embodiment, post-translation modifications such as phosphorylation of tau at specific residues can be measure and quantified in addition to the cleaved fragment of tau. Alternatively, or in addition, Aβ (e.g. Aβ42/40), ApoE, or any other protein of interest may be identified and/or quantified either by processing a portion of the biological sample in parallel, by depleting the protein of interest from the biological sample prior to utilization in the methods disclosed herein, or by depleting the protein of interest from the biological sample during the sample processing steps disclosed herein.

The biological sample, suitable internal standards, and the steps of, purifying tau, optionally depleting one or more proteins, optionally cleaving purified tau with a protease, and detecting the cleaved fragment of tau are described in more detail below.

d. Biological Sample

Suitable biological samples include a blood sample or a cerebrospinal fluid (CSF) sample obtained from a subject. In some embodiments, the subject is a human. A human subject may be waiting for medical care or treatment, may be under medical care or treatment, or may have received medical care or treatment. In various embodiments, a human subject may be a healthy subject, a subject at risk of developing a neurodegenerative disease, a subject with signs and/or symptoms of a neurodegenerative disease, or a subject diagnosed with a neurodegenerative disease. In further embodiments, the neurodegenerative disease may be a tauopathy. In specific examples, the tauopathy may be Alzheimer's disease (AD), progressive supranuclear palsy (PSP), corticobasal degeneration (CBD), or frontotemporal lobar degeneration (FTLD). In other embodiments, the subject is a laboratory animal. In a further embodiment, the subject is a laboratory animal genetically engineered to express human tau and optionally one or more additional human protein (e.g., human Aβ, human ApoE, etc.).

CSF may have been obtained by lumbar puncture with or without an indwelling CSF catheter. Multiple blood or CSF samples contemporaneously collected from the subject may be pooled. Blood may have been collected by venipuncture with or without an intravenous catheter, or by a finger stick (or the equivalent thereof). Once collected, blood or CSF samples may have been processed according to methods known in the art (e.g., centrifugation to remove whole cells and cellular debris; use of additives designed to stabilize and preserve the specimen prior to analytical testing; etc.). Blood or CSF samples may be used immediately or may be frozen and stored indefinitely. Prior to use in the methods disclosed herein, the biological sample may also have been modified, if needed or desired, to include protease inhibitors, isotope labeled internal standards, detergent(s) and chaotropic agent(s), and/or to optionally deplete other analytes (e.g. proteins peptides, metabolites).

The size of the sample used can and will vary depending upon the sample type, the health status of the subject from whom the sample was obtained, and the analytes to be analyzed (in addition to tau). CSF samples volumes may be about 0.01 mL to about 5 mL, or about 0.05 mL to about 5 mL. In a specific example, the size of the sample may be about 0.05 mL to about 1 mL CSF. Plasma sample volumes may be about 0.01 mL to about 20 mL.

In some embodiments, a single sample is obtained from a subject. Alternatively, samples may be obtained from a subject over time. As such, more than one sample may be collected from a subject over time. For instance, 2, 3, 4, 5, 6, 7, 8, 9, 10, 11, 12, 13, 14, 15, 16 or more samples may be collected from a subject over time. When more than on sample is collected from a subject over time, samples may be collected every 1, 2, 3, 4, 5, 6, 7, 8, 9, 10, 11, 12 or more days. In some embodiments, a sample is collected a month apart, 3 months apart, 6 months apart, 1 year apart, 2 years apart, 5 years apart, 10 years apart, 20 years apart or more.

e. Isotope-Labeled, Internal Tau Standard

Isotope-labeled tau may be used as an internal standard to account for variability throughout sample processing and optionally to calculate an absolute concentration. Generally, an isotope-labeled, internal tau standard is added before significant sample processing, and it can be added more than once if needed.

Multiple isotope-labeled internal tau standards are described herein. All have a heavy isotope label incorporated into at least one amino acid residue. One or more full-length isoforms may be used. Alternatively, or in addition, tau isoforms with post-translational modifications and/or peptide fragments of tau may also be used, as is known in the art. Generally speaking, the labeled amino acid residues that are incorporated should increase the mass of the peptide without affecting its chemical properties, and the mass shift resulting from the presence of the isotope labels must be sufficient to allow the mass spectrometry method to distinguish the internal standard (IS) from endogenous tau analyte signals. As shown herein, suitable heavy isotope labels include, but are not limited to 2H, 13C, and 15N. Typically, about 1-10 ng of internal standard is usually sufficient.

f. Purifying Tau

Another step of the methods disclosed herein comprises purifying tau, in particular an endogenously cleaved and proteolytically cleaved fragments of tau. In some examples, the cleaved fragment of tau is N-terminal-independent, mid-domain-independent, and/or C-terminal-independent. The purified tau may be partially purified or completely purified.

In some embodiments, a method of the present disclosure comprises purifying tau by affinity purification. Affinity purification refers to methods that enrich for a protein of interest by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. Purifying a cleaved fragment of tau by affinity purification comprises contacting a sample comprising an endogenously cleaved fragment tau with a suitable immobilized ligand, one or more wash steps, and elution of the cleaved fragment tau from the immobilized ligand.

In some embodiments, a method of the present disclosure comprises purifying a cleaved fragment tau by affinity purification using at least one epitope-binding agent that specifically binds to an epitope within amino acids 225-258 of tau-441, inclusive, or within amino acids 235-258 of tau-441, inclusive, or within amino acids 235-242, inclusive, (or within similarly defined regions for other full-length isoforms). In various embodiments, one, two, three or more epitope-binding agents may be used. When two or more epitope-binding agents are used, they may be used sequentially or simultaneously. Non-limiting examples of suitable epitope-binding agents are disclosed herein. In various embodiments, one, two, three or more epitope-binding agents may be used. When two or more epitope-binding agents are used, they may be used sequentially or simultaneously.

In each of the above embodiments, the epitope-binding agent may comprise an antibody or an aptamer. In some embodiments, an epitope-binding agent that specifically binds to an epitope within a cleaved fragment tau is HJ32.11, or is an epitope-binding agent that binds the same epitope as HJ32.11 and/or competitively inhibits HJ32.11.

In some embodiments, a method of the present disclosure comprises purifying tau by solid phase extraction. Purifying tau by solid phase extraction comprises contacting a sample comprising tau with a solid phase comprising a sorbent that adsorbs tau, one or more wash steps, and elution of tau from the sorbent. Suitable sorbents include reversed-phase sorbents. Suitable reversed phase sorbents are known in the art and include, but are not limited to alkyl-bonded silicas, aryl-bonded silicas, styrene/divynlbenzene materials,N-vinylpyrrolidone/divynlbenzene materials. In an exemplary embodiment, the reversed phase material is a polymer comprising N-vinylpyrrolidone and divinylbenzene or a polymer comprising styrene and divinylbenzene. In an exemplary embodiment, a sorbent is Oasis HLB (Waters). Prior to contact with the supernatant comprising tau, the sorbent is typically preconditioned per manufacturer's instructions or as is known in the art (e.g., with a water miscible organic solvent and then the buffer comprising the mobile phase). In addition, the supernatant may be optionally acidified, as some reversed-phase materials retain ionized analytes more strongly than others. The use of volatile components in the mobile phases and for elution is preferred, as they facilitate sample drying. In exemplary embodiments, a wash step may comprise the use of a liquid phase comprising about 0.05% v/v trifluoroacetic acid (TFA) to about 1% v/v TFA, or an equivalent thereof. In some examples, the wash may be with a liquid phase comprising about 0.05% v/v to about 0.5% v/v TFA or about 0.05% v/v to about 0.1% v/v TFA. In some examples, the wash may be with a liquid phase comprising about 0.1% v/v to about 1.0% v/v TFA or about 0.1% v/v to about 0.5% v/v TFA. Bound tau is then eluted with a liquid phase comprising about 20% v/v to about 50% v/v acetonitrile (ACN), or an equivalent thereof. In some examples, tau is may be eluted with a liquid phase comprising about 20% v/v to about 40% v/v ACN, or about 20% v/v to about 30% v/v ACN. In some examples, tau is may be eluted with a liquid phase comprising about 30% v/v to about 50% v/v ACN, or about 30% v/v to about 40% v/v ACN. The eluate may be dried by methods known in the art (e.g., vacuum drying (e.g., speed-vac), lyophilization, evaporation under a nitrogen stream, etc.).

g. Depleting One or More Proteins

Methods of the present disclosure may comprise a step wherein one or more protein is depleted from a sample. The term “deplete” means to diminish in quantity or number. Accordingly, a sample depleted of a protein may have any amount of the protein that is measurably less than the amount in the original sample, including no amount of the protein.

Protein(s) may be depleted from a sample by a method that specifically targets one or more protein, for example by affinity depletion, solid phase extraction, or other method known in the art. Targeted depletion of a protein, or multiple proteins, may be used in situations where downstream analysis of that protein is desired (e.g., identification, quantification, analysis of post-translation modifications, etc.). For instance, Aβ peptides may be identified and quantified by methods known in the art following affinity depletion of Aβ with a suitable epitope-binding agent. As another non-limiting example, apolipoprotein E (ApoE) status may be determined by methods known in the art following affinity depletion of ApoE and identification of the ApoE isoform. Targeted depletion may also be used to isolate other proteins for subsequent analysis including, but not limited to, apolipoprotein J, synuclein, soluble amyloid precursor protein, alpha-2 macroglobulin, S100B, myelin basic protein, an interleukin, TNF, TREM-2, TDP-43, YKL-40, VILIP-1, NFL, prion protein, pNFH, and DJ-1. Targeted depletion of certain tau proteins is also used herein to enrich for other tau proteins and/or eliminate proteins that cofound the mass spectrometry analysis. For instance, in certain embodiments of the present disclosure, N-terminal tau proteins, mid-domain tau proteins, and/or long MTBR-tau proteins are depleted from a sample prior to further sample processing for analysis. Downstream analysis of the depleted tau proteins may or may not occur, but both options are contemplated by the methods of the present disclosure.

In some embodiments, targeted depletion may occur by affinity depletion. Affinity depletion refers to methods that deplete a protein of interest from a sample by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. The molecule may also be a polymer or other material that selectively absorbs a protein of interest. As a non-limiting example, polyhydroxymethylene substituted by fat oxethylized alcohol (e.g., PHM-L LIPOSORB, Sigma Aldrich) may be used to selectively absorb lipoproteins (including ApoE) from serum. Two or more affinity depletion agents may be combined to sequentially or simultaneously deplete multiple proteins.

In some embodiments, a method of the present disclosure comprises affinity depleting one or more protein from a sample using at least one epitope-binding agent that specifically binds to an epitope within amino acids 1 to 243 of tau-441, inclusive (or within a similarly defined region for 0N or IN isoforms). In various embodiments, one, two, three or more epitope-binding agents may be used. When two or more epitope-binding agents are used, they may be used sequentially or simultaneously.

In some embodiments, a method of the present disclosure comprises affinity depleting one or more protein from a sample using an epitope-binding agent that specifically binds to an epitope within the N-terminus of tau (e.g., amino acids 1 to 103 of tau-441, inclusive), and an epitope-binding agent that specifically binds to an epitope within the mid-domain of tau (e.g., amino acids 104 to 243 of tau-441, inclusive). The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive, and an epitope-binding agent that specifically binds to an epitope within amino acids 104 to 243 of tau-441, inclusive (or within similarly defined regions for 0N or IN isoforms). The epitope-binding agents may be used sequentially or simultaneously.

In some embodiments, a method of the present disclosure comprises affinity depleting one or more protein from a sample using an epitope-binding agent that specifically binds to an epitope within amino acids 1 to 103 of tau-441, inclusive (or within a similarly defined region for 0N or 1N isoforms); an epitope-binding agent that specifically binds to an epitope within amino acids 104 to 243 of tau-441, inclusive (or within a similarly defined region for 0N or IN isoforms); and an epitope binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive (or within a similarly defined region for 0N or IN isoforms); an epitope-binding agent that specifically binds to an epitope within amino acids 104 to 243 of tau-441, inclusive (or within a similarly defined region for 0N or IN isoforms); and an epitope binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 103 of tau-441, inclusive (or within a similarly defined region for 0N or IN isoforms); and an epitope-binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive (or within a similarly defined region for 0N or IN isoforms); and an epitope-binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 104 to 243 of tau-441, inclusive (or within a similarly defined region for 0N or IN isoforms); and an epitope binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 260 to 441 of tau-441, inclusive.

In each of the above embodiments, the epitope binding agent may comprise an antibody or an aptamer. In some embodiments, the epitope-binding agent that specifically binds to amyloid beta is HJ5.1, or is an epitope-binding agent that binds the same epitope as HJ5.1 and/or competitively inhibits HJ5.1. In some embodiments, the epitope-binding agent that specifically binds to that specifically binds to an epitope within amino acids 1 to 103 of tau-441, inclusive, is HJ8.5, or is an epitope-binding agent that binds the same epitope as HJ8.5 and/or competitively inhibits HJ8.5. In some embodiments, the epitope-binding agent that specifically binds to that specifically binds to an epitope within amino acids 104 to 221 of tau-441, inclusive, is Tau1, or is an epitope-binding agent that binds the same epitope as Tau1 and/or competitively inhibits Tau1. In some embodiments, the epitope-binding agent that specifically binds to that specifically binds to an epitope within amino acids 260 to 441 of tau-441, inclusive, is HJ34.8, or is an epitope-binding agent that binds the same epitope as HJ34.8 and/or competitively inhibits HJ43.8. In some embodiments, the epitope-binding agent that specifically binds to that specifically binds to an epitope within amino acids 260 to 441 of tau-441, inclusive, is HJ34.8, or is an epitope-binding agent that binds the same epitope as 77G7, RD3, RD4, UCB1017, or PT76, is an epitope-binding agent that binds the same epitope as HJ34.8 and/or competitively inhibits 77G7, RD3, RD4, UCB1017, or PT76 described in Vandermeeren et al., J Alzheimers Dis, 2018, 65:265-281, and antibodies E2814 and 7G6 described in Roberts et al., Acta Neuropathol Commun, 2020, 8:13, as well as other epitope-binding agents that specifically bind the same epitopes as those antibodies. Methods for identifying epitopes to which an antibody specifically binds, and assays to evaluate competitive inhibition between two antibodies, are known in the art.

Alternatively, protein(s) may be depleted from a sample by a more general method, for example by ultrafiltration or protein precipitation with an acid, an organic solvent or a salt. Generally speaking, these methods are used to reliably reduce high abundance and high molecular weight proteins, which in turn enriches for low molecular weight and/or low abundance proteins and peptides (e.g., tau, Aβ, etc.).

In some embodiments, proteins may be depleted from a sample by precipitation. Briefly, precipitation comprises adding a precipitating agent to a sample and thoroughly mixing, incubating the sample with precipitating agent to precipitate proteins, and separating the precipitated proteins by centrifugation or filtration. The resulting supernatant may then be used in downstream applications. The amount of the reagent needed may be experimentally determined by methods known in the art. Suitable precipitating agents include perchloric acid, trichloroacetic acid, acetonitrile, methanol, and the like. In an exemplary embodiment, proteins are depleted from a sample by acid precipitation. In a further embodiment, proteins are depleted from a sample by acid precipitation using perchloric acid.

As a non-limiting example, proteins may be depleted from a sample by acid precipitation using perchloric acid. As used herein, “perchloric acid” refers to 70% perchloric acid unless otherwise indicated. In some embodiments, perchloric acid is added to a final concentration of about 1% v/v to about 15% v/v. In other embodiments, perchloric acid is added to a final concentration of about 1% v/v to about 10% v/v. In other embodiments, perchloric acid is added to a final concentration of about 1% v/v to about 5% v/v. In other embodiments, perchloric acid is added to a final concentration of about 3% v/v to about 15% v/v. In other embodiments, perchloric acid is added to a final concentration of about 3% v/v to about 10% v/v. In other embodiments, perchloric acid is added to a final concentration of about 3% v/v to about 5% v/v. In other embodiments, perchloric acid is added to a final concentration of 3.5% v/v to about 15% v/v, 3.5% v/v to about 10% v/v, or 3.5% v/v to about 5% v/v. In other embodiments, perchloric acid is added to a final concentration of about 3.5% v/v. Following addition of the perchloric acid, the sample is mixed well (e.g., by a vortex mixer) and held at a cold temperature, typically for about 10 minutes or longer, to facilitate precipitation. For example, samples may be held for about 10 minutes to about 60 minutes, about 20 minutes to about 60 minutes, or about 30 minutes to about 60 minutes. In other example, samples may be held for about 15 minutes to about 45 minutes, or about 30 minutes to about 45 minutes. In other examples, samples may be held for about 15 minutes to about 30 minutes, or about 20 minutes to about 40 minutes. In other examples, samples are held for about 30 minutes. The sample is then centrifuged at a cold temperature to pellet the precipitated protein, and the supernatant (i.e., the acid soluble fraction), comprising soluble tau, is transferred to a fresh vessel. As used in the above context, a “cold temperature” refers to a temperature of 10° C. or less. For instance, a cold temperature may be about 1° C., about 2° C., about 3° C., about 4° C., about 5° C., about 6° C., about 7° C., about 8° C., about 9° C., or about 10° C. In some embodiments, a narrower temperature range may be preferred, for example, about 3° C. to about 5° C., or even about 4° C. In certain embodiments, a cold temperature may be achieved by placing a sample on ice.

Two or more methods from one or both of the above approaches may be combined to sequentially or simultaneously deplete multiple proteins. For instance, one or more proteins may be selectively depleted (targeted depletion) followed by depletion of high abundance/molecular weight proteins. Alternatively, high abundance/molecular weight proteins may be first depleted followed by targeted depletion of one or more proteins. In still another alternative, high abundance/molecular weight proteins may be first depleted followed by a first round of targeted depletion of one or more proteins and then a second round of targeted depletion of one or more different protein(s) than targeted in the first round. Other iterations will be readily apparent to a skilled artisan.

h. Cleaving Purified Tau with a Protease

Another step of the methods disclosed herein optionally comprises cleaving purified tau with a protease. Cleaving purified tau with a protease comprises contacting a sample comprising purified tau with a protease under conditions suitable to digest tau. When affinity purification is used, digestion may occur after eluting tau from the immobilized ligand or while tau is bound. Suitable proteases include but are not limited to trypsin, Lys-N, Lys-C, Asp-C, Arg-N, and Arg-C. For detecting very short MTBR-tau243, the proteases comprise Arg-C. The resultant cleavage product is a composition comprising proteolytic peptides of tau. When the protease is trypsin, the resultant cleavage product comprises tryptic peptides of tau. Following proteolytic cleavage, the resultant cleavage product may be desalted by solid phase extraction.

i. Detection and Quantifying

Another step of the method disclosed herein comprises detecting the amount of cleaved fragments of tau from the processed biological sample. All suitable method of detecting an amount of tau protein are contemplated within the scope of the disclosure. Methods of detecting and quantifying tau protein are described in detail below.

### (1) LC-MS

The step of detecting and quantifying comprises performing liquid chromatography-mass spectrometry (LC-MS) with a sample comprising peptides of tau to detect and measure the concentration of at least one peptide of tau. Thus, in practice, the disclosed methods use one or more tau peptide to detect and measure the amount of tau protein present in the biological sample.

The proteolytic peptides of tau that indicate the presence of endogenously cleaved fragments of tau may comprise one or more of the peptides listed in Table 1. When using an alternative enzyme for digestion, the resulting proteolytic peptides may differ slightly but can be readily determined by a person of ordinary skill in the art. Without wishing to be bound by theory, it is believed that a variation in the amount of a cleaved fragments of tau peptides between two biological samples of the same type reflects a difference in the cleaved fragments of tau that make up those biological samples. As disclosed herein, the amounts of certain proteolytic peptides of cleaved fragments of tau, as well ratios of certain proteolytic peptides of tau, may provide clinically meaningful information to guide treatment decisions. Thus, methods that allow for detection and quantification of cleaved fragments of tau have utility in the diagnosis and treatment of many neurodegenerative diseases.

Proteolytic peptides of tau may be separated by a liquid chromatography system interfaced with a high-resolution mass spectrometer. Suitable LC-MS systems may comprise a <1.0 mm ID column and use a flow rate less than about 100 μl/min. In preferred embodiments, a nanoflow LC-MS system is used (e.g., about 50-100 μm ID column and a flow rate of <1 μL/min, preferably about 100-800 nL/min, more preferably about 200-600 nL/min). In an exemplary embodiment, an LC-MS system may comprise a 0.05 mM ID column and use a flow rate of about 400 nL/min.

Tandem mass spectrometry may be used to improve resolution, as is known in the art, or technology may improve to achieve the resolution of tandem mass spectrometry with a single mass analyzer. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer, Orbitrap Fusion™ Lumos™ Mass Spectrometer, Orbitrap Tribrid™ Eclipse™ Mass Spectrometer, Q Exactive Mass Spectrometer, each from ThermoFisher Scientific). In an exemplary embodiment, an LC-MS system may comprise a mass spectrometer selected from Orbitrap Fusion™ Tribrid™ Mass Spectrometer, Orbitrap Fusion™ Lumos™ Mass Spectrometer, Orbitrap Tribrid™ Eclipse™ Mass Spectrometer, or a mass spectrometer with similar or improved ion-focusing and ion-transparency at the quadrupole. Suitable mass spectrometry protocols may be developed by optimizing the number of ions collected prior to analysis (e.g., AGC setting using an orbitrap) and/or injection time. In an exemplary embodiment, a mass spectrometry protocol outlined in the Examples is used.

### (2) Immunoassays

In another embodiment, cleaved fragments of tau can be measured and quantified by immunoassay. In a specific embodiment, cleaved fragments of tau are detected and quantified using an ELISA.

Methods for assessing an amount of protein expression using epitope binding agent-based methods are known in the art and all suitable methods for assessing an amount of protein known to one of skill in the art are contemplated within the scope of the present disclosure.

Thus, in some embodiments, the method to assess an amount of tau protein is an epitope binding agent-based method. In general, an epitope binding agent-based method of assessing an amount of protein expression comprises contacting a sample comprising a polypeptide with an epitope binding agent specific for the polypeptide under conditions effective to allow for formation of a complex between the epitope binding agent and the polypeptide. Epitope binding agent-based method may occur in solution, or the epitope binding agent or sample may be immobilized on a solid surface. Non-limiting examples of suitable surfaces include microtiter plates, test tubes, beads, resins, and other polymers.

An epitope binding agent may be attached to the substrate in a wide variety of ways, as will be appreciated by those in the art. The epitope binding agent may either be synthesized first, with subsequent attachment to the substrate, or may be directly synthesized on the substrate. The substrate and the epitope binding agent may be derivatized with chemical functional groups for subsequent attachment of the two. For example, the substrate may be derivatized with a chemical functional group including, but not limited to, amino groups, carboxyl groups, oxo groups or thiol groups. Using these functional groups, the epitope binding agent may be attached directly using the functional groups or indirectly using linkers.

The epitope binding agent may also be attached to the substrate non-covalently. For example, a biotinylated epitope binding agent may be prepared, which may bind to surfaces covalently coated with streptavidin, resulting in attachment. Alternatively, an epitope binding agent may be synthesized on the surface using techniques such as photopolymerization and photolithography. Additional methods of attaching epitope binding agents to solid surfaces and methods of synthesizing biomolecules on substrates are well known in the art, i.e. VLSIPS technology from Affymetrix (e.g., see U.S. Pat. No. 6,566,495, and Rockett and Dix, Xenobiotica 30 (2): 155-177, both of which are hereby incorporated by reference in their entirety).

Contacting the sample with an epitope binding agent under effective conditions for a period of time sufficient to allow formation of a complex generally involves adding the epitope binding agent composition to the sample and incubating the mixture for a period of time long enough for the epitope binding agent to bind to any antigen present. After this time, the complex will be washed and the complex may be detected by any method well known in the art. Methods of detecting the epitope binding agent-polypeptide complex are generally based on the detection of a label or marker. The term “label”, as used herein, refers to any substance attached to an epitope binding agent, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni2+, Flag tags, myc tags, heavy metals, and enzymes (including alkaline phosphatase, peroxidase, and luciferase). Methods of detecting an epitope binding agent-polypeptide complex based on the detection of a label or marker are well known in the art.

In some embodiments, an epitope binding agent-based method is an immunoassay. Immunoassays can be run in a number of different formats. Generally speaking, immunoassays can be divided into two categories: competitive immmunoassays and non-competitive immunoassays. In a competitive immunoassay, an unlabeled analyte in a sample competes with labeled analyte to bind an antibody. Unbound analyte is washed away and the bound analyte is measured. In a noncompetitive immunoassay, the antibody is labeled, not the analyte. Non-competitive immunoassays may use one antibody (e.g. the capture antibody is labeled) or more than one antibody (e.g. at least one capture antibody which is unlabeled and at least one “capping” or detection antibody which is labeled.) Suitable labels are described above.

In an embodiment, the epitope binding agent method is an immunoassay. In another embodiment, the epitope binding agent method is selected from the group consisting of an enzyme linked immunoassay (ELISA), a fluorescence based assay, a dissociation enhanced lanthanide fluoroimmunoassay (DELFIA), a radiometric assay, a multiplex immunoassay, and a cytometric bead assay (CBA). In some embodiments, the epitope binding agent-based method is an enzyme linked immunoassay (ELISA). In other embodiments, the epitope binding agent-based method is a radioimmunoassay. In still other embodiments, the epitope binding agent-based method is an immunoblot or Western blot. In alternative embodiments, the epitope binding agent-based method is an array. In another embodiment, the epitope binding agent-based method is flow cytometry. In different embodiments, the epitope binding agent-based method is immunohistochemistry (IHC). IHC uses an antibody to detect and quantify antigens in intact tissue samples. The tissue samples may be fresh-frozen and/or formalin-fixed, paraffin-embedded (or plastic-embedded) tissue blocks prepared for study by IHC. Methods of preparing tissue block for study by IHC, as well as methods of performing IHC are well known in the art.

### 4. Uses of Cleaved Fragments of Tau Measurements

The present disclosure also encompasses the use of measurements of cleaved fragments of tau in blood or CSF as biomarkers of pathological features and/or clinical symptoms of tauopathies to diagnose, stage, choose treatments appropriate for a given disease stage, and modify a given treatment regimen (e.g., change a dose, switch to a different drug or treatment modality, etc.). The pathological feature may be an aspect of tau pathology (e.g., amount of tau deposition, presence/absence of a post-translational modification, amount of a post-translation modification, etc.). Alternatively, or in addition to tau deposition, a pathological feature may be tau-independent. For instance, amyloid beta (Aβ) deposition in the brain or in arteries of the brain when the tauopathy is Alzheimer's disease. The clinical symptom may be dementia, as measured by a clinically validated instrument (e.g., MMSE, CDR-SB, etc.), or any other clinical symptom associated with the tauopathy.

Accordingly, in one aspect, the present disclosure provides a method of measuring tauopathy-related pathology in a subject, the method comprising quantifying one or more cleaved fragments of tau in a biological sample obtained from a subject, such as a blood sample or a CSF sample, wherein the amount(s) of the quantified cleaved fragments of tau is/are a representation of tauopathy-related pathology in the brain of the subject. The tauopathy may be a 3R-tauopathy, a mixed 3R/4R-tauopathy, or a 4R-tauopathy. The disease-related pathology may be tau deposition, tau post-translational modification, amyloid plaques in the brain and/or arteries of the brain, or other pathological feature known in the art. The subject may or may not have clinical symptoms of the tauopathy. A cleaved fragment of tau quantified may comprise one or more of the amino acid sequences in Table 1. In further embodiments, two or more cleaved fragments of tau are quantified.

In another aspect, the present disclosure provides a method of diagnosing a tauopathy in a subject, the method comprising quantifying one or more cleaved fragments of tau in a biological sample obtained from a subject, such as a blood sample or a CSF sample, and diagnosing a tauopathy when the quantified cleaved fragment of tau is/are about 1.56 or above, where σ is the standard deviation defined by the normal distribution measured in a control population that does not have clinical signs or symptoms of a tauopathy and is amyloid negative as measured by PET imaging and/or Aβ42/40 measurement in CSF. The tauopathy may be a 3R-tauopathy, a mixed 3R/4R-tauopathy, or a 4R-tauopathy. The subject may or may not have clinical symptoms of disease. A cleaved fragment of tau quantified may comprise one or more of the amino acid sequences in Table 1. In further embodiments, two or more cleaved fragments of tau are quantified.

In another aspect, the present disclosure provides a method of measuring tauopathy disease stability in a subject, the method comprising quantifying one or more cleaved fragment of tau in a first biological sample obtained from a subject and then in a second biological sample obtained from the same subject at a later time (e.g., weeks, months or years later), and calculating the difference between the quantified cleaved fragment of tau between the samples, wherein a statistically significant increase in the quantified cleaved fragment of tau in the second sample indicates disease progression, a statistically significant decrease in the quantified cleaved fragment of tau in the second sample indicates disease improvement, and no change indicates stable disease. The tauopathy may be a 3R-tauopathy, a mixed 3R/4R-tauopathy, or a 4R-tauopathy. The subject may or may not have clinical symptoms of disease, and may or may not be receiving a tau therapy. In some examples, a tau therapy is administered one or more times to the subject in the period of time between collection of the first and second biological sample, and the measure of disease stability is an indication of the effectiveness, or lack thereof, of the tau therapy. In preferred embodiments, a cleaved fragment of tau quantified comprises one or more of the amino acid sequences in Table 1. In further embodiments, two or more cleaved fragments of tau are quantified.

In another aspect, the present disclosure provides a method of treating a subject with a tauopathy, the method comprising quantifying a cleaved fragment of tau in a biological sample obtained from a subject, such as a blood sample or a CSF sample; and providing a tau therapy to the subject to improve a measurement of disease-related pathology or a clinical symptom, wherein the subject has a quantified cleaved fragment of tau at least 1 standard deviation, preferably at least 1.3 standard deviations, more preferably at least 1.5 standard deviations or even more preferably at least 2 standard deviations, above or below the mean of a control population (i.e., differs by 1σ, 1.3σ, 1.5σ, or 1.5σ, respectively, where σ is the standard deviation defined by the normal distribution measured in a control population does not have clinical signs or symptoms of a tauopathy and that is amyloid negative as measured by PET imaging and/or Aβ42/40 measurement in CSF. In addition to using a threshold (e.g. at least 1 standard deviation above or below the mean), in some embodiments the extent of change above or below the mean may be used as criteria for treating a subject. The tauopathy may be a 3R-tauopathy, a mixed 3R/4R-tauopathy, or a 4R-tauopathy. The measurement of disease-related pathology may be tau deposition as measured by PET imaging, tau post-translational modification as measured by mass spectrometry or other suitable method, amyloid plaques in the brain or arteries of the brain as measured by PET imaging, amyloid plaques as measured by Aβ42/40 in CSF, or other pathological features known in the art. The clinical symptom may be dementia, as measured by a clinically validated instrument (e.g., MMSE, CDR-SB, etc.) or other clinical symptoms known in the art for 3R- and 4R-tauopathies. In preferred embodiments, a cleaved fragment of tau quantified comprises one or more of the amino acid sequences in Table 1. In further embodiments, two or more cleaved fragments of tau are quantified. Many tau therapies target a specific pathophysiological change. For instance, Aβ targeting therapies are generally designed to decrease Aβ production, antagonize Aβ aggregation or increase brain Aβ clearance; tau targeting therapies are generally designed to alter tau phosphorylation patterns, antagonize tau aggregation (general antagonism of tau or antagonism of a specific tau isoform), or increase NFT clearance; a variety of therapies are designed to reduce CNS inflammation or brain insulin resistance; etc. However, not all tauopathies share the same pathophysiological changes. Therefore, the efficacy of these various tau therapies can be improved by administering them to subjects that are correctly identified as having a tau pathology, including determining the stage of disease the subject is in thereby more efficiently altering tau phosphorylation patterns, antagonizing tau aggregation, or increasing NFT clearance based on the specific pathological state of the subject.

Tau species which contain MTBR-tau243 (in reference to tau-441), as disclosed herein, are diversly present in biological fluids such as CSF and blood, although not all tau species which contain MTBR-tau243 show disease specific correlations. The present disclosure provides cleaved fragments of tau containing MTBR-tau243 which directly correlate with the Tau-PET status. Thus, detecting these cleaved fragments of tau in biological fluids provides a sensitive and specific methods for measuring tau pathology. Suitable biological samples are described herein. Assaying these cleaved fragments of tau include purifying the same from a biological sample. Suitable methods for purifying cleaved fragments of tau are described herein, as are epitope-binding agents.

In each of the above aspects, suitable cleaved fragments of tau may include, but are not limited to, cleaved fragments of tau comprising or consisting of one or more of the amino sequences in Table 1, or combinations thereof.

Exemplary uses of cleaved fragments of tau can serve to illustrate various aspects discussed above, but such discussions do not limit the scope of the invention. Cleaved fragments of tau are described in detail in the Examples. Generally speaking, these tau fragments contain MTBR-tau243 and are cleaved endogenously at their C-terminus. Measuring the amount of endogenously cleaved fragments of tau is one means by which to measure, in a given sample, the amount of this specific group of tau species. As shown in the Examples, increases in the amount of blood and/or CSF cleaved fragments of tau recapitulate direct measures of tau deposition in the brain associated with Alzheimer's disease (AD). Stated another way, the amount of blood and/or CSF cleaved fragments of tau is a representation of AD-related pathology (e.g., tau deposition in the brain). These amounts can therefore be used to measure AD-related pathology, to determine a subject's tau status, and to diagnose AD stage in subjects, among other things. The amount of blood and/or CSF cleaved fragments of tau also recapitulates changes measured during clinical stages of AD, for example as defined by the results of tau-PET testing. Accordingly, the amount of cleaved fragments of tau can also aid to diagnose and stage AD in subjects across the entire disease spectrum (e.g., pre-clinical to clinical). After diagnosing and/or staging disease, treatments may then be provided to the subject to decrease, or prevent any further increase, in the amount of cleaved fragments of tau in blood and/or CSF and/or to decrease, or prevent any further increase, of another clinical sign or symptom of AD. Choice of treatment may be further guided by knowledge of the specific disease stage that is informed by the amount of cleaved fragments of tau—for instance, therapies designed to prevent Aβ deposition, reverse Aβ deposition, prevent tau deposition, reverse tau deposition, and improve clinical signs of disease would be used in subjects with different, albeit potentially overlapping, amount of cleaved fragments of tau.

In a specific embodiment, the present disclosure provides a method of measuring Alzheimer disease (AD)-related pathology in a subject, the method comprising providing a CSF or blood sample obtained from a subject, wherein the CSF or blood sample is purified for a cleaved fragment of tau; and quantifying, in the sample, the cleaved fragment of tau. In some embodiments, the cleaved fragment of tau comprises one or more of the peptides having an amino acid sequence in Table 1, where the C-terminal amino acid represents the last amino acid on the C-terminus of the peptide, or a combination thereof, wherein the amount of the cleaved fragment of tau, or their ratios, is a representation of AD-related pathology in a brain of a subject.

In another specific embodiment, the present disclosure provides a method of measuring Alzheimer disease (AD)-related tau deposition in a brain of a subject, the method comprising providing a CSF or blood sample obtained from a subject, wherein the CSF or blood sample is purified for a cleaved fragment of tau; and quantifying, in the sample, the cleaved fragment of tau. In some embodiments, the cleaved fragment of tau comprises one or more of the peptides having an amino acid sequence in Table 1, where the C-terminal amino acid represents the last amino acid on the C-terminus of the peptide, or a combination thereof, wherein the amount of the cleaved fragment of tau is a representation of AD-related tau deposition in a brain of a subject.

In another specific embodiment, the present disclosure provides a method of diagnosing Alzheimer's disease, the method comprising providing a CSF or blood sample obtained from a subject, and quantifying, in the sample, the cleaved fragment of tau. In some embodiments, the cleaved fragment of tau comprises one or more of the peptides having an amino acid sequence in Table 1, where the C-terminal amino acid represents the last amino acid on the C-terminus of the peptide, or a combination thereof. In some embodiments, the method comprises quantifying p-tau (e.g., tau181, p-tau205, p-tau217, and p-tau231), Aβ (e.g., Aβ42/40), or a combination thereof. In some embodiments, a subject is diagnosed with Alzheimer's disease when the quantified cleaved fragment of tau differs by about 1.5σ or more from the mean of a control population, where σ is the standard deviation defined by the normal distribution measured in a control population does not have clinical signs or symptoms of a tauopathy and that is amyloid negative as measured by PET imaging (for instance by PiB-PET SUVR as described in Ann Neurol 2016; 80:379-387) and/or Aβ42/40 measurement in CSF (for instance, a cutoff value for CSF Aβ42/40 calculated from PiB-PET SUVR (Ann Neurol 2016; 80:379-387) that maximizes sensitivity %+Specificity %).

In another specific embodiment, the present disclosure provides a method of measuring Alzheimer disease (AD) progression in a subject, the method comprising providing a first CSF or blood sample and a second CSF or blood sample, wherein each sample is obtained from a single subject, and each sample is purified for a cleaved fragment of tau; and for each sample, quantifying the cleaved fragment of tau. In some embodiments, the cleaved fragment of tau comprises one or more of the peptides having an amino acid sequence in Table 1, where the C-terminal amino acid represents the last amino acid on the C-terminus of the peptide, or a combination thereof. In some embodiments, the method comprises p-tau or Aβ, or a combination thereof. The method further comprising calculating the difference between the quantified cleaved fragment of tau in the second sample and the first sample, wherein a statistically significant increase in the quantified cleaved fragment of tau in the second sample indicates progression of the subject's Alzheimer's disease.

In another specific embodiment, the present disclosure provides a method of measuring tau deposition in a brain in a subject, the method comprising providing a CSF or blood sample obtained from a subject, wherein the CSF or blood sample is purified for a cleaved fragment of tau; and quantifying, in the sample, the cleaved fragment of tau. In some embodiments, the cleaved fragment of tau comprises one or more of the peptides having an amino acid sequence in Table 1, where the C-terminal amino acid represents the last amino acid on the C-terminus of the peptide, or a combination thereof, wherein the amount of the cleaved fragment of tau, or their ratios, is a representation of tau deposition in a brain of a subject.

The specific embodiments that follow are directed to methods that comprise a method of measuring a cleaved fragment of tau in a biological sample can be found in Section II, incorporated herein by reference.

In each of the preceding methods additional biomarkers can be detected and measured to aid in staging and/or determining the pathology of the subject and/or disease progression of the subject. For example, the method may include detecting the amount of Aβ. In some embodiments an Aβ42/40 value is determined to aid in measuring amyloid plaque specific abnormalities. Aβ42/40 values which deviate from a normal control population without amyloid pathology begin to occur some 20 years before symptom on set and proved a high sensitivity measure of these changes. In addition to or in the place of an Aβ measurement, each of the preceding methods may include detecting the amount of tau phosphorylation at specific residues. In some embodiments, phosphorylation of tau at 217, 181, 231, 205, 153, 111, 208, and/or any combination thereof, are determined to aid in measuring amyloid plaque specific abnormalities. Aβ42/40 is the first to change, then p-tau217/181/231 shortly thereafter and those are associated with amyloid plaques. Then about 10 years later, p-tau205 is increased and associated with both Aβ plaques and tau tangles, similar to total-tau (n-terminal to mid-domain tau). In some embodiments, a composite value of ptau phosphorylation and Aβ42/40 value can be measured and determined. For example, pT217 x Aβ42/40 value is determined to aid in measuring amyloid plaque specific abnormalities and is found to be highly sensitive and specific. Finally, about 20 years after Aβ42/40 abnormalities, cleaved fragment of tau as disclosed herein is increased and correlates with tau pathology only (not amyloid pathology). Thus, each marker when measured and combined in the methods disclosed herein provides a more precise ability to determine a subjects stage, pathology and/or disease progression which has not been available prior to the present disclosure. Aβ42/40 is a measure of pre-amyloid plaque abnormalities; Aβ42/40 & ptau217% abnormal is a measure of amyloid plaque abnormalities, ptau205 is a measure of amyloid plaque plus neurodegeneration; and cleaved fragment of tau as disclosed herein is a measure of tau tangles and clinical onset of dementia.

Alternatively or in addition to the above, additional markers such as a measurement of total tau, in any of the above embodiments, a ratio calculated from the measured phosphorylation level(s), or a ratio calculated from the measured phosphorylation level(s) and total tau, may be used. Mathematical operations other than a ratio may also be used. For instance, the examples use of a cleaved fragment of tau as disclosed herein, Aβ values, and/or site-specific tau phosphorylation values can be used in various statistical models (e.g., linear regressions, LME curves, LOESS curves, etc.) in conjunction with other known biomarkers (e.g. MAPT status, APOE ε4 status, age, sex, cognitive test scores, functional test scores, etc.). Selection of measurements and choice of mathematical operations may be optimized to maximize specificity of the method. For instance, diagnostic accuracy may be evaluated by area under the ROC curve and in some embodiments, an ROC AUC value of 0.7 or greater is set as a threshold (e.g., 0.7, 0.75, 0.8, 0.85, 0.9, 0.95, etc.).

Brain amyloid plaques in humans are routinely measured by amyloid-positron emission tomography (PET). For instance, 11C-Pittsburgh compound B (PiB) PET imaging of cortical Aβ-plaques is commonly used to detect Aβ-plaque pathology. The standard uptake value ratio (SUVR) of cortical PiB-PET reliably identifies significant cortical Aβ-plaques and is used to classify subjects as PIB positive (SUVR ≥1.25) or negative (SUVR <1.25). Accordingly, in the above embodiments, a control population without brain amyloid plaques as measured by PET imaging may refer to a population of subjects that have a cortical PiB-PET SUVR <1.25. Other values of PiB binding (e.g., mean cortical binding potential) or analyses of regions of interest other than the cortical region may also be used to classify subjects as PIB positive or negative. Other PET imaging agents may also be used.

In another specific embodiment, the present disclosure provides a method of treating a subject in need thereof or a method of selecting a therapeutic agent for a subject in need thereof, the methods comprising providing a CSF or blood sample obtained from a subject, wherein the CSF or blood sample is purified for a cleaved fragment of tau; and quantifying, in the sample, the cleaved fragment of tau. In some embodiments, the cleaved fragment of tau comprises one or more of the peptides having an amino acid sequence in Table 1, where the C-terminal amino acid represents the last amino acid on the C-terminus of the peptide, or a combination thereof, wherein the amount of the cleaved fragment of tau, or their ratios, is a representation of tau deposition in a brain of a subject. In some embodiments, the subject is administered a therapeutic agent when the amount of cleaved fragment of tau differs by about 1.5σ or more from the mean from a healthy control population, where σ is the standard deviation defined by the normal distribution measured in a control population that does not have clinical signs or symptoms of a tauopathy and is amyloid negative as measured by PET imaging and/or Aβ42/40 measurement in CSF, and wherein the amount of the quantified cleaved fragment of tau is a representation of tau pathology in a brain of a subject. In some embodiments, administering a treatment to the subject to alter tau pathology alters or stabilizes the amount of the quantified cleaved fragment of tau.

In another specific embodiment, the present disclosure provides a method treating a subject in need thereof or a method of selecting a therapeutic agent for a subject in need thereof, the methods comprising providing a CSF or blood sample obtained from a subject, wherein the CSF or blood sample is purified for a cleaved fragment of tau; quantifying, in the sample, the cleaved fragment of tau; and administering a treatment to the subject to alter tau pathology, wherein the amount of cleaved fragment of tau differs by about 1.5σ or more from the mean from a healthy control population, where σ is the standard deviation defined by the normal distribution measured in a control population that does not have clinical signs or symptoms of a tauopathy and is amyloid negative as measured by PET imaging and/or Aβ42/40 measurement in CSF, and wherein the amount of the quantified cleaved fragment of tau is a representation of tau pathology in a brain of a subject. In some embodiments, the cleaved fragment of tau comprises one or more of the peptides having an amino acid sequence in Table 1, where the C-terminal amino acid represents the last amino acid on the C-terminus of the peptide, or a combination thereof, wherein the amount of the cleaved fragment of tau, or their ratios, is a representation of tau deposition in a brain of a subject.

The treatment may comprise one or more of lecanemab, donanemab, AADvac1, ACI-3024, ACI-35, APNmAb005, ASN51, AZP2006, BIIB076, BIIB080, BIIB113, Bepranemab, Dasatinib+Quercetin, E2814, Epothilone D, Gosuranemab, JNJ-63733657, LM™, LY3372689, Lu AF87908, MK-2214, NIO752, OLX-07010, PNT001, PRX005, RG7345, Rember™, Semorinemab, TPI 287, Tideglusib, Tilavonemab, Zagotenemab, an anti-tau monoclonal antibody, an anti-tau anti-sense oligonucleotide, an anti-tau small interfering RNA, an tau production inhibitor, and a tau active vaccine.

In some embodiments the treatment is a pharmaceutical composition comprising a cholinesterase inhibitor, an N-methyl D-aspartate (NMDA) antagonist, an antidepressant (e.g., a selective serotonin reuptake inhibitor, an atypical antidepressant, an aminoketone, a selective serotonin and norepinephrine reuptake inhibitor, a tricyclic antidepressant, etc.), a gamma-secretase inhibitor, a beta-secretase inhibitor, an anti-Aβ antibody (including antigen-binding fragments, variants, or derivatives thereof), an anti-tau antibody (including antigen-binding fragments, variants, or derivatives thereof), an anti-TREM2 antibody (including antigen-binding fragments, variants or derivatives thereof, a TREM2 agonist, stem cells, dietary supplements (e.g. lithium water, omega-3 fatty acids with lipoic acid, long chain triglycerides, genistein, resveratrol, curcumin, and grape seed extract, etc.), an antagonist of the serotonin receptor 6, a p38alpha MAPK inhibitor, a recombinant granulocyte macrophage colony-stimulating factor, a passive immunotherapy, an active vaccine (e.g. CAD106, AF20513, etc.), a tau protein aggregation inhibitor (e.g. TRx0237, methylthionimium chloride, etc.), a therapy to improve blood sugar control (e.g., insulin, exenatide, liraglutide pioglitazone, etc.), an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a β-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a Glutaminyl-peptide cyclotransferase inhibitor, a selective inhibitor of APP production, a monoamine oxidase B inhibitor, a glutamate receptor antagonist, a AMPA receptor agonist, a nerve growth factor stimulant, a HMG-COA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, a statin, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A. In some embodiments the treatment is a pharmaceutical composition comprising a tau targeting therapy, including but not limited to active pharmaceutical ingredients that alter tau phosphorylation patterns, antagonize tau aggregation, or increase clearance of pathological tau isoforms and/or aggregates. In some embodiments, the treatment is an anti-AB antibody, an anti-tau antibody, an anti-TREM2 antibody, a TREM2 agonist, a gamma-secretase inhibitor, a beta-secretase inhibitor, a kinase inhibitor, a phosphatase activator, a vaccine, or a tau protein aggregation inhibitor.

In some embodiments, the subject is administered a therapeutic agent or a therapeutic agent is selected which prevents amyloid deposition from increasing when the detected Aβ42/40 value deviates from the mean of a healthy control population and optionally pTau217/tau217, ptau205/tau205, pTau181, pTau 231, and/or a cleaved fragment of the present disclosure do not significantly deviate from the mean of a healthy control population. In an exemplary embodiment, the detected Aβ42/40 value deviates significantly below the mean of a healthy control population. In some embodiments, the subject is administered a therapeutic agent or a therapeutic agent is selected which prevents amyloid deposition from increasing and/or reduces a subject's existing plaque load when the detected Aβ42/40 value and pTau217/tau217 (or pTau217 x Aβ42/40 composite value) significantly deviates from the mean of a healthy control population and optionally ptau205/tau205, and/or a cleaved fragment of the present disclosure do not significantly deviate from the mean of a healthy control population. In an exemplary embodiment, the detected Aβ42/40 value deviates significantly below the mean of a healthy control population, the detected pTau217/tau217 is above the mean of a healthy control population and/or the pTau217 x Aβ42/40 composite value is above the mean of a healthy control population. In some embodiments, the subject is administered a therapeutic agent or a therapeutic agent is selected which prevents amyloid deposition from increasing and/or reduces a subject's existing plaque load and/or treats or prevents neurodegeneration and/or prevents Tau tangles or related pathology when the detected Aβ42/40 value, pTau217/tau217 (or pTau217 x Aβ42/40 composite value), and pTau205/tau205 value significantly deviate from the mean of a healthy control population and optionally a cleaved fragment of the present disclosure do not significantly deviate from the mean of a healthy control population. In an exemplary embodiment, the detected Aβ42/40 value deviates significantly below the mean of a healthy control population, the detected pTau217/tau217 is above the mean of a healthy control population and/or the pTau217 x Aβ42/40 composite value is above the mean of a healthy control population and/or the pTau205/tau205 value is above the mean of a healthy control population. In some embodiments, the subject is administered a therapeutic agent or a therapeutic agent is selected which prevents amyloid deposition from increasing and/or reduces a subject's existing plaque load and/or treats or prevents neurodegeneration and/or prevents tau tangles from increasing and/or reduces a subject's existing tangles or related pathology when the detected Aβ42/40 value, pTau217/tau217 (or pTau217 x Aβ42/40 composite value), pTau205/tau205 and a cleaved fragment of the present disclosure significantly deviate from the mean of a healthy control population. In an exemplary embodiment, the detected Aβ42/40 value deviates significantly below the mean of a healthy control population, the detected pTau217/tau217 is above the mean of a healthy control population and/or the pTau217 x Aβ42/40 composite value is above the mean of a healthy control population and/or the pTau205/tau205 value is above the mean of a healthy control population and/or a cleaved fragment of the present disclosure value is above the mean of a health control population.

In each of the above embodiments, a pharmaceutical composition administered to a subject may comprise an imaging agent. Non-limiting examples of imaging agents include functional imaging agents (e.g. fluorodeoxyglucose, etc.) and molecular imaging agents (e.g., Pittsburgh compound B, florbetaben, florbetapir, flutemetamol, radionuclide-labeled antibodies, etc.).

Another aspect of the present disclosure is a method of selecting a subject into a clinical trial, in particular a clinical trial for an Aβ or tau therapy, provided all other criteria for the clinical trial have been met. In one embodiment, a method of a method of selecting a subject into a clinical trial may comprise (a) providing a CSF or blood sample obtained from a subject, wherein the CSF or blood sample is purified for a cleaved fragment of tau; (b) quantifying, in the sample, the cleaved fragment of tau and (c) selecting the subject into a clinical trial for an Aβ therapy when the cleaved fragment of tau value is about the same as a healthy control population and the subject's Aβ42/40 value is below the man of a healthy control population. In some embodiments, the cleaved fragment of tau comprises one or more of the peptides having an amino acid sequence in Table 1, where the C-terminal amino acid represents the last amino acid on the C-terminus of the peptide, or a combination thereof, wherein the amount of the cleaved fragment of tau, or their ratios, is a representation of tau deposition in a brain of a subject.

In another embodiment, a method of selecting a subject into a clinical trial may comprise (a) providing a CSF or blood sample obtained from a subject, wherein the CSF or blood sample is purified for a cleaved fragment of tau; (b) quantifying, in the sample, the cleaved fragment of tau and (c) excluding the subject into a clinical trial an Aβ therapy when the cleaved fragment of tau value is above the mean of a healthy control population and the subject's Aβ42/40 value is about the same or below the mean of a healthy control population. In some embodiments, the cleaved fragment of tau comprises one or more of the peptides having an amino acid sequence in Table 1, where the C-terminal amino acid represents the last amino acid on the C-terminus of the peptide, or a combination thereof, wherein the amount of the cleaved fragment of tau, or their ratios, is a representation of tau deposition in a brain of a subject.

In another embodiment, a method of selecting a subject into a clinical trial, in particular a clinical trial for a tau therapy, provided all other criteria for the clinical trial have been met. In one embodiment, a method of a method of selecting a subject into a clinical trial may comprise (a) providing a CSF or blood sample obtained from a subject, wherein the CSF or blood sample is purified for a cleaved fragment of tau; (b) quantifying, in the sample, the cleaved fragment of tau and (c) selecting the subject into a clinical trial a tau therapy when the cleaved fragment of tau value is above the mean of a healthy control population and optionally when the subject's Aβ42/40 value is about the same or below the mean of a healthy control population. In some embodiments, the cleaved fragment of tau comprises one or more of the peptides comprising an amino acid sequence in Table 1, where the C-terminal amino acid represents the last amino acid on the C-terminus of the peptide, or a combination thereof, wherein the amount of the cleaved fragment of tau, or their ratios, is a representation of tau deposition in a brain of a subject.

In another embodiment, a method of selecting a subject into a clinical trial, in particular a clinical trial for a tau therapy, provided all other criteria for the clinical trial have been met. In one embodiment, a method of a method of selecting a subject into a clinical trial may comprise (a) providing a CSF or blood sample obtained from a subject, wherein the CSF or blood sample is purified for a cleaved fragment of tau; (b) quantifying, in the sample, the cleaved fragment of tau and (c) excluding the subject into a clinical trial a tau therapy when the cleaved fragment of tau value is about the same as the mean of a healthy control population. In some embodiments, the cleaved fragment of tau comprises one or more of the peptides comprising an amino acid sequence in Table 1, where the C-terminal amino acid represents the last amino acid on the C-terminus of the peptide, or a combination thereof, wherein the amount of the cleaved fragment of tau, or their ratios, is a representation of tau deposition in a brain of a subject. The phrase “a control population without brain amyloid plaques as measured by PET imaging” is defined above.

Alternatively or in addition to using a measurement of a cleaved fragment of tau as disclosed herein, site-specific tau phosphorylation, optionally with a measurement of total tau, in any of the above embodiments, a ratio calculated from the measured phosphorylation level(s), or a ratio calculated from the measured phosphorylation level(s) and total tau, may be used. A ratio calculated from the measured phosphorylation level(s) may be a ratio between pT181 and pT205, pT217 and pT205, or pT181 and pT217. A ratio calculated from the measured phosphorylation level(s) and total tau may be a ratio between pT181 and total tau, p-T205 and total tau, or pT217 and total tau. Mathematical operations other than a ratio may also be used. For instance, the examples use site-specific tau phosphorylation values in various statistical models (e.g., linear regressions, LME curves, LOESS curves, etc.) in conjunction with other known biomarkers (e.g. APOE ε4 status, age, sex, cognitive test scores, functional test scores, etc.).

The design of clinical trials for AD and tau therapies can be greatly aided by the methods disclosed herein. Many clinical trials are designed to test the efficacy of imaging agents or therapeutic agents that target a specific pathophysiological change which occurs prior to the onset of AD symptoms. As discussed above in Section III, the efficacy of these various agents can be improved by administering the agents to subjects that have certain site-specific tau phosphorylation levels, as measured by methods disclosed herein and illustrated. Similarly, clinical trials selecting subjects with symptoms of Aβ pathology or tau only pathology would also benefit from being able to accurately discriminate an enrollee's pathology in order to determine if efficacy is associated with a particular disease state. Accordingly, measuring tau phosphorylation levels as described herein prior to selecting a subject in a clinical trial, in particular into a treatment arm of a clinical trial, may result in smaller trials and/or improved outcomes. In some instances, methods described herein may be developed and used as a companion diagnostic for a therapeutic agent.

In each of the above embodiments, a subject may be enrolled into a treatment arm of the clinical trial. The “treatment” is defined above. Subjects enrolled in the treatment arm of a clinical trial may be administered a pharmaceutical composition. In some embodiments, a pharmaceutical composition may comprise an imaging agent. Non-limiting examples of imaging agents include functional imaging agents (e.g. fluorodeoxyglucose, etc.) and molecular imaging agents (e.g., Pittsburgh compound B, florbetaben, florbetapir, flutemetamol, radionuclide-labeled antibodies, etc.). Alternatively, a pharmaceutical composition may comprise an active pharmaceutical ingredient. Non-limiting examples of active pharmaceutical ingredients include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Aβ antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-COA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A.

In each of the above embodiments, a subject may or may not be symptomatic. An “asymptomatic subject,” as used herein, refers to a subject that does not show any signs or symptoms of a tauopathy. Alternatively, a subject may exhibit signs or symptoms (e.g., memory loss, misplacing things, changes in mood or behavior, etc.,) but not show sufficient cognitive or functional impairment for a clinical diagnosis. A symptomatic or an asymptomatic subject may have Aβ amyloidosis; however, prior knowledge of Aβ amyloidosis is not a requisite for treatment. In still further embodiments, a subject may have AD. In any of the aforementioned embodiments, a subject may carry one of the gene mutations known to cause an inherited tauopathy. In alternative embodiments, a subject may not carry a gene mutation known to cause an inherited tauopathy.

### 5. Kits

Also provided are kits. Such kits can include an agent or composition described herein and, in certain embodiments, instructions for administration or measuring an endogenous fragment of tau. Such kits can facilitate performance of the methods described herein. When supplied as a kit, the different components of the composition can be packaged in separate containers and admixed immediately before use. Components include, but are not limited to systems, assays, epitope biding agents, reagents, internal standards, or software. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or from a randomized group of subjects. A reference value can be used in place of a control or reference sample, which was previously obtained from a healthy subject or a group of healthy subject. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### EXAMPLES

The present invention has multiple aspects, illustrated by the following non-limiting examples.

### Example 1

### CSF MTBR-Tau243 Biomarker of Tau Tangle Pathology

Given the growing interest in tau-targeted therapeutics for Alzheimer's disease (AD), there is a critical need for reliable and specific biomarkers of insoluble, aggregated tau to understand AD pathophysiology and to evaluate the effects of treatments¹. Positron emission tomography (PET) with radio-ligands that bind to fibrillar forms of tau reflect the burden of insoluble AD-specific tau aggregates in the brain, including neurofibrillary tangles (NFT) and neuropil threads²⁻⁶. Tau-PET imaging studies have shown that insoluble tau aggregates are strongly associated with cognitive decline even during the early pre-symptomatic stages of AD⁷ and tau-PET is the most accurate prognostic marker of AD available today⁸. However, PET imaging is highly expensive and needs a complex infrastructure, which reduces its use to only highly specialized centers. In contrast, fluid biomarkers are less expensive and are more clinically accessible. The most widely used fluid biomarkers of tau are N-terminal or mid-domain total tau (t-tau) and phosphorylated tau species resulting from cleavage near residue 224 of tau^{9,10}, including tau phosphorylated at residues 181, 217, and 231 (p-tau181, p-tau217, and p-tau231)¹¹⁻¹⁷. But, these biomarkers are strongly associated with increasing burden of amyloid plaques more than insoluble tau aggregates¹⁸⁻²⁰. For instance, plasma and CSF concentrations of these p-tau species are already increased in preclinical AD many years before widespread insoluble tau aggregates in the neocortex are observed²¹⁻²⁴. Further, recent clinical trials have demonstrated substantial reductions of CSF or plasma concentrations of t-tau, p-tau181, and p-tau217²⁵⁻²⁸ in response to anti-amyloid passive immunotherapies which substantially remove amyloid plaques. Neuropathological and imaging studies have also reported strong associations between these fluid biomarkers and amyloid plaques^{19,20,29}. In addition, animal studies have found that CSF t-tau and p-tau are increased in mouse models with amyloid β (Aβ) pathology, even when no aggregated tau pathology is observed^{23,30-32}. Taken together, these findings indicate that plasma and CSF concentrations of N-terminal to mid-domain t-tau and p-tau do not directly represent insoluble tau aggregates, but rather reflect a response to amyloid plaque pathology. Thus, there is currently no fluid biomarker that specifically reflects AD-related tau pathology.

In this study, we therefore evaluated a novel CSF biomarker of insoluble tau aggregates. Importantly, tau species that contain the microtubule-binding region (MTBR-tau) are a major component of insoluble tau aggregates in the brain³³⁻³⁷, but these fragments have been poorly investigated as candidate biomarkers. In an initial study, with a small sample size of controls and AD patients (n=35), we showed preliminary results that MTBR-tau was present in human CSF and that a specific MTBR-tau species containing residue 243 (MTBR-tau243) was strongly associated with tau-PET and disease progression³³. Here, we expanded these results to two large independent sporadic AD cohorts, the Swedish BioFINDER-2 study and the Charles F. and Joanne Knight Alzheimer Disease Research Center (Knight ADRC), covering the whole AD continuum, with available amyloid- and tau-PET images. In this study, we compared the performance of MTBR-tau243 to other CSF phosphorylated tau measures, including p-tau181, p-tau205, p-tau217, and p-tau231 phosphorylation occupancies (i.e., % p-tau to total tau ratio) which are also reported as biomarkers to recapitulate AD pathologies^{21,29}, and we showed that MTBR-tau243 was the fluid biomarker most strongly associated with tau-PET. We also investigated the proportion of variation in CSF biomarker levels explained by amyloid- and tau-PET measures of pathology. Then, we evaluated longitudinal CSF biomarker changes to investigate their rate of change based on the presence or absence of amyloid and tau pathologies to indicate which are increasing with amyloid vs. tau pathologies. Finally, we assessed whether prediction of continuous AD-related measures could be improved by the combination of multiple biomarkers, and found that MTBR-tau243, together with p-tau205, could optimally predict tau-PET measures and cognitive impairment.

### Results

### Participants Characteristics

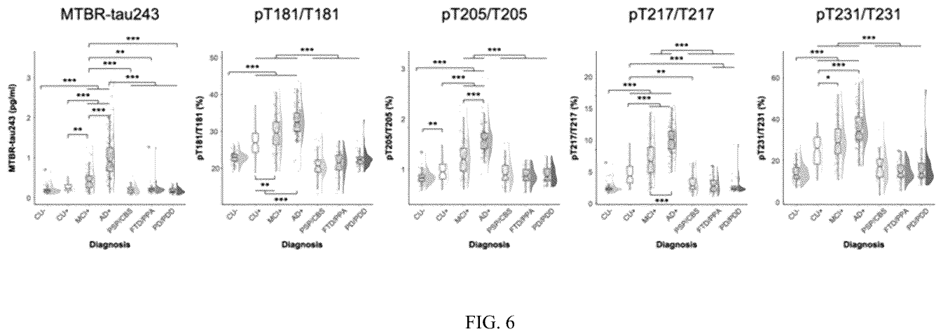

The BioFINDER-2 cohort included 448 individuals, the majority of whom had cognitive impairment (281, 63%): 81 cognitively unimpaired Aβ negative (CU−), 79 cognitively unimpaired Aβ positive (CU+), 90 Aβ positive with mild cognitive impairment (MCI+), 102 Aβ positive with AD dementia (AD+) and 96 with other dementias (non-AD) (Table 2). The average age was 70.9±8.4 years (mean±standard deviation), 221 (49.3%) were women, and 258 (57.6%) were APOE ε4 carriers. The Knight ADRC cohort included 219 individuals, most of whom were cognitively unimpaired (171, 78%): 83 CU−, 88 CU+, 35 very mild AD, and 13 AD+. The average age was 71.2±6.6 years, 112 (51.1%) were women, and 96 (43.8%) were APOE ε4 carriers (Table 3). CSF biomarkers were measured in the BioFINDER-2 and the Knight ADRC cohorts, including MTBR-tau243 concentration, as well as the phosphorylation occupancy at different tau residues (percent pT181/T181, pT205/T205, pT217/T217, and pT231/T231). The phosphorylation occupancy represents the percentage of soluble tau phosphorylated at a certain amino acid position (see methods for details), which is a more specific measure of phosphorylation not confounded with total tau concentrations, and superior to the corresponding pTau concentration in prediction of abnormal Aβ status^{29,38} In FIG. 6 and Table 4, we compared the CSF levels of all biomarkers in all diagnostic groups in the BioFINDER-2 cohort. We observed that MTBR-tau243 concentrations were not increased in other non-AD tauopathies such as progressive supranuclear palsy (PSP) or frontotemporal dementia (FTD), thus suggesting a high specificity for AD-related tau. Further, we did not observe any significant difference between MTBR-tau243 concentrations in CU+ compared to CU−. Of note, we found that two outliers (one in CU− and the other in FTD) which had very high levels of MTBR-tau243 were MAPT R406W mutation carriers who were amyloid negative, but clearly tau-PET positive (indicated in FIG. 6).

Table 4 shows associations between CSF biomarkers and Aβ-PET and tau-PET. Linear regression models were used to access the associations between CSF biomarkers and amyloid-PET (Centiloids) and tau-PET (SUVR) in Braak I-IV ROI adjusting for age and sex. Amyloid-positive participants were selected based on CSF Aβ42/40 previously validated cutoffs (CSF Aβ42/40<0.08 in BioFINDER-2 and CSF Aβ42/40<0.0673 in Knight ADRC). P comparison (p comp.) was calculated to assess differences between the strongest association (Ref.) and each of the other CSF biomarkers using bootstrapping (n=500) from adjusted B. Significant p comparison (<0.05) suggests weaker associations. Abbreviations: Aβ, amyloid; CI, confidence interval; CSF, cerebrospinal fluid; MTBR, microtubule binding region; PET, positron emission tomography; SUVR, standardized uptake value ratio.

TABLE 2
BioFINDER-2 participants characteristics

| Overall | | CU− | | CU+ | | MCI+ | | AD+ | | Non-AD | |
|---|---|---|---|---|---|---|---|---|---|---|---|
| n= | 448 | n= | 81 | n= | 79 | n= | 90 | n= | 102 | n= | 96 |

Demographics

| | | | | | | | | | | | | |
|---|---|---|---|---|---|---|---|---|---|---|---|---|
| Age, years | 448 | 70.9 | 81 | 69.9 | 79 | 70.5 | 90 | 71.7 | 102 | 72.5 | 96 | 69.8 |
| | | (8.44) | | (9.66) | | (9.54) | | (7.34) | | (6.86) | | (8.71) |
| Women, n (%) | 448 | 221 | 81 | 40 | 79 | 40 | 90 | 38 | 102 | 57 | 96 | 46 |
| | | 49.3% | | 49.4% | | 50.6% | | 42.2% | | 55.9% | | 47.9% |
| APOE-ε4carriers, n | 447 | 258 | 81 | 27 | 79 | 58 | 89 | 65 | 102 | 76 | 96 | 32 |
| (%) | | 57.6% | | 33.3% | | 73.4% | | 72.2% | | 74.5% | | 33.3% |
| Years of education | 443 | 12.2 | 81 | 12 | 79 | 12.2 | 89 | 12.5 | 101 | 11.9 | 93 | 12.5 |
| | | (3.74) | | (3.21) | | (3.36) | | (4.59) | | (3.85) | | (3.54) |

CSF Aβ measures

| | | | | | | | | | | | | |
|---|---|---|---|---|---|---|---|---|---|---|---|---|
| CSF Aβ42/40 | 427 | 0.0687 | 81 | 0.108 | 79 | 0.0553 | 85 | 0.0492 | 100 | 0.0447 | 82 | 0.0922 |
| | | (0.0293) | | (0.0132) | | (0.0134) | | (0.0130) | | (0.0113) | | (0.0217) |
| CSF Aβ42/40 | 427 | 290 | 81 | 0 | 79 | 79 | 90 | 90 | 102 | 102 | 82 | 26 |
| positivity, n (%) | | 64.7% | | 0% | | 100% | | 100% | | 100% | | 27.1% |

Amyloid-and Tau-PET measures

| | | | | | | | | | | | | |
|---|---|---|---|---|---|---|---|---|---|---|---|---|
| Amyloid-PET, | 268 | 38.4 | 81 | 4.48 | 79 | 41.6 | 88 | 71.6 | 7 | 115 | 11 | 12.6 |
| Centiloids | | (44.4) | | (9.26) | | (36.5) | | (35) | | (23.3) | | (25.1) |
| Amyloid-PET | 268 | 148 | 81 | 2 | 79 | 55 | 88 | 79 | 7 | 7 | 11 | 3 |
| positivity. n (%) | | 33.0% | | 2.5% | | 69.6% | | 87.8% | | 6.9% | | 3.1% |
| Tau-PET Braak I-IV, | 443 | 1.53 | 81 | 1.17 | 79 | 1.23 | 90 | 1.51 | 101 | 2.4 | 92 | 1.17 |
| SUVR | | (0.612) | | (0.0888) | | (0.206) | | (0.447) | | (0.599) | | (0.124) |
| Tau-PET positivity. n | 443 | 162 | 81 | 1 | 79 | 11 | 90 | 45 | 101 | 101 | 92 | 4 |
| (%) | | 36.2% | | 1.2% | | 119% | | 50.0% | | 99.0% | | 4.2% |

CSFtau by mass spectrometry

| | | | | | | | | | | | | |
|---|---|---|---|---|---|---|---|---|---|---|---|---|
| pT181/T181(%) | 448 | 26.9 | 81 | 22.8 | N | 27.2 | 90 | 29.5 | 102 | 32.6 | 96 | 21.8 |
| | | (5.63) | | (1.63) | | (3.83) | | (4.85) | | (4.14) | | (3.61) |
| pT205/T205(%) | 448 | 1.14 | 81 | 0.789 | 79 | 0.973 | 90 | 1.24 | 102 | 1.7 | 96 | 0.863 |
| | | (0.452) | | (0.144) | | (0.284) | | (0.41) | | (0.322) | | (0.239) |
| pT217/T217(%) | 448 | 6.56 | 81 | 2.78 | 79 | 5.71 | 90 | 8.02 | 102 | 11.9 | 96 | 3.44 |
| | | (4.12) | | (0.831) | | (2.51) | | (3.24) | | (2.53) | | (1.7) |
| pT231/T231(%) | 448 | 12.3 | 81 | 7.09 | 79 | 12.7 | 90 | 14.8 | 102 | 17.9 | 96 | 8.14 |
| | | (5.69) | | (2.15) | | (4.16) | | (4.59) | | (4.46) | | (3.61) |
| MTBR-tau243 (pg/ml) | 448 | 0.445 | 81 | 0.192 | 79 | 0.281 | 90 | 0.449 | 102 | 0.992 | 96 | 0.207 |
| | | (0.424) | | (0.0885) | | (0.165) | | (0.279) | | (0.502) | | (0.139) |

Cognitive measures

| | | | | | | | | | | | | |
|---|---|---|---|---|---|---|---|---|---|---|---|---|
| MMSE, mean (SD) | 447 | 25.8 | 81 | 29.1 | 79 | 28.8 | 90 | 26.8 | 101 | 19.8 | 96 | 25.8 |
| | | (4.57) | | (1.13) | | (1.33) | | (1.88) | | (4.44) | | (3.97) |

Data are presented as mean (SD).
Values in square brackets indicate the % in total number within the group.
Abbreviations: AD+, Alzheimer's disease dementia amyloid positive; CSF, cerebrospinal fluid; CU−, cognitively unimpaired amyloid negative; CU+, cognitively unimpaired amyloid positive; MCI+, mild cognitive impairment amyloid positive; MMSE, Mini-Mental State Examination; MTBR, microtubule binding region; non-AD, non-Alzheimer's disease dementia; PET, positron emission tomography; SUVR, standardized uptake value ratio.

TABLE 3
Knight ADRC participants characteristics

| | ALL | | CU− | | CU+ | |
|---|---|---|---|---|---|---|
| Characteristic | n= | 219 | n= | 83 | n= | 88 |

Demographics

| Age, years | 219 | 71.2 | (6.55 | 83 | 68.5 | (6.55) | 88 | 71.4 | (11.3) |
|---|---|---|---|---|---|---|---|---|---|
| Women, n (%) | 219 | 112, 51.1% | | 83 | 39, 47.0% | | 88 | 50, 56.8% | |
| Race | 219 | 9/206/4 | | 83 | 7/76/0 | | 88 | 2/83/3 | |
| (Black/white/Other), n | | | | | | | | | |
| (%) | | | | | | | | | |
| APOE-ε4carriers, n (%) | 217 | 93, 43.8% | | 83 | 17, 20.4% | | 87 | 49, 56.3% | |
| Years of education | 219 | 16.3 | (2.81) | 83 | 16.6 | (2.81 | 88 | 16.4 | (2.39) |

CSF Aβ measures

| | | | | | | | | | |
|---|---|---|---|---|---|---|---|---|---|
| CSF Aβ42/Aβ40 | 219 | 0.0574 | (0.0094) | 83 | 0.0895 | (0.0094) | 88 | 0.0481 | (0.0182) |
| CSF Aβ42/40 positivity, n | 219 | 136 | (62.1%) | 83 | 0 | (0%) | 88 | 88 | (100%) |
| (%) | | | | | | | | | |

Amyloid-and tau-PET measures

| Amyloid-PET, Centiloid | 219 | 41.8 | (28) | 83 | 9.17 | (28) | 88 | 48.6 | (29.7) |
|---|---|---|---|---|---|---|---|---|---|
| Amyloid-PET status (n, | 219 | 123, 56.2% | | 83 | 4, 4.8% | | 88 | 72, 81.8% | |
| % positive) | | | | | | | | | |
| Interval between LP and | 219 | 0.115 | (0.29) | 83 | 0.101 | (0.29) | 88 | 0.0986 | (0.214) |
| amyloid-PET | | | | | | | | | |
| Tau-PET Braak I-IV, | 219 | 1.18 | (0.289) | 83 | 1.14 | (0.289) | 88 | 1.19 | (0.102) |
| SUVR | | | | | | | | | |
| Tau-PET positivity, n (%) | 219 | 26, 16.4% | | 83 | 1, 0% | | 88 | 6, 6.8% | |
| Interval between LP and | 219 | 0.112 | (0.17) | 83 | 0.112 | (0.17) | 88 | 0.11 | (0.203) |
| tau-PET (years) | | | | | | | | | |

CSF tau by mass spectrometry

| | | | | | | | | | |
|---|---|---|---|---|---|---|---|---|---|
| pT181/T181(%) | 219 | 27.8 | (4.31) | 83 | 24.5 | (4.31) | 88 | 29.9 | (6.57) |
| pT205/T205(%) | 219 | 1.04 | (0.65) | 83 | 0.87 | (0.65) | 88 | 1.08 | (0.46) |
| pT217/T217(%) | 219 | 5.78 | (4.84) | 83 | 3.06 | (4.84) | 88 | 7.68 | (4.77) |
| pT231/T231(%) | 184 | 14.7 | (8.13) | 64 | 4.76 | (8.13) | 78 | 22.2 | (19.6) |
| MTBR-tau243 (pg/ml) | 219 | 0.207 | (0.35) | 83 | 0.14 | (0.35) | 88 | 0.23 | (0.116) |

Cognitive measures

| | | | | | | | | | |
|---|---|---|---|---|---|---|---|---|---|
| MMSE | 219 | 28.5 | (2.73) | 83 | 29.5 | (2.73) | 88 | 29.2 | (1.12) |

| | Very mild AD | | | AD+ | | |
|---|---|---|---|---|---|---|
| Characteristic | n= | 35 | | n= | 13 | |
| Demographics | | | | | | |
| Age, years | 35 | 75.5 | (6.55) | 13 | 74.9 | (7.2) |
| Women, n (%) | 35 | 17, 48.6% | | 13 | 6, 46.2% | |
| Race | 35 | 0/34/1 | | 13 | 0/13/0 | |
| (Black/white/Other), n | | | | | | |
| (%) | | | | | | |
| APOE-ε4carriers, n (%) | 34 | 22, 64.7% | | 13 | 8, 61.5% | |
| Years of education | 35 | 15.4 | (2.81) | 13 | 15.5 | (2.7) |
| CSF Aβ measures | | | | | | |
| CSF Aβ42/Aβ40 | 35 | 0.0447 | (0.0094) | 13 | 0.0378 | (0.0123) |
| CSF Aβ42/40 positivity, n | 35 | 35 | (100%) | 13 | 13 | (100%) |
| (%) | | | | | | |
| Amyloid-and tau-PET measures | | | | | | |
| Amyloid-PET, Centiloid | 35 | 77.9 | (28) | 13 | 107 | (37.1) |
| Amyloid-PET status (n, | 35 | 34, 97.1% | | 13 | 13, 100% | |
| % positive) | | | | | | |
| Interval between LP and | 35 | 0.14 | (0.29) | 13 | 0.186 | (0.222) |
| amyloid-PET | | | | | | |
| Tau-PET Braak I-IV, | 35 | 1.31 | (0.289) | 13 | 1.57 | (0.526) |
| SUVR | | | | | | |
| Tau-PET positivity, n (%) | 35 | 17, 48.6% | | 13 | 13, 100% | |
| Interval between LP and | 35 | 0.164 | (0.17) | 13 | 0.0821 | (0.257) |
| tau-PET (years) | | | | | | |
| CSF tau by mass spectrometry | | | | | | |
| pT181/T181(%) | 35 | 33.7 | (4.31) | 13 | 31.8 | (4.37) |
| pT205/T205(%) | 35 | 1.65 | (0.65) | 13 | 1.98 | (0.54) |
| pT217/T217(%) | 35 | 11.5 | (4.84) | 13 | 12.9 | (3.14) |
| pT231/T231(%) | 31 | 25.5 | (8.13) | 8 | 41.2 | (17.4) |
| MTBR-tau243 (pg/ml) | 35 | 0.451 | (0.35) | 13 | 0.701 | (0.335) |
| Cognitive measures | | | | | | |
| MMSE | 35 | 27.1 | (2.73) | 13 | 22.2 | (3.18) |

Abbreviations: AD+, Alzheimer's disease dementia amyloid positive; CSF, cerebrospinal fluid; CU−, cognitively unimpaired amyloid negative; CU+, MMSE, Mini-Mental State Examination; MTBR, microtubule binding region; PET, positron emission tomography; SUVR, standardized uptake value ratio. Parenthesis in rows: standard deviation.

TABLE 4
Associations between CSF biomarkers and AB-PET and tau-PET

| | BioFinder-2 | | | | Knight ADRC | | | |
|---|---|---|---|---|---|---|---|---|
| Biomarker | β std[95% CI] | p (β) | p (comp.) | R² | β std[95% CI] | p (β) | p (comp.) | R² |

Aβ-PET
All participants

| | | | | | | | | |
|---|---|---|---|---|---|---|---|---|
| pT1B1/T1B1 | 0.66 | <0.001 | <0.001 | 0.47 | 0.67 | <0.001 | <0.001 | 0.47 |
| | [0.57, 0.75] | | | | [0.56, 0.78] | | | |
| p1205/T205 | 0.66 | <0001 | <0.001 | 0.47 | 0.69 | <0.001 | <0.001 | 0.49 |
| | [0.57, 0.75] | | | | [0.58, 0.78] | | | |
| pT217/T217 | 0.81 | <0.001 | Ref. | 0.68 | 0.87 | <0.001 | Ref | 0.74 |
| | [0.74, 0.88] | | | | [0.79, 0.95] | | | |
| p1231/T231 | 0.75 | <0.001 | 0.008 | 0.60 | 0.68 | <0.001 | <0.001 | 0.45 |
| | [0.68, 0.83] | | | | [051, 0.73] | | | |
| MTBR-tau243 | 0.56 | <0.001 | <0.001 | 0.33 | 0.61 | <0101 | <0.001 | 0.37 |
| | [0.46, 0.66] | | | | [0.49, 0.74] | | | |

Aβ-positive participants

| | | | | | | | | |
|---|---|---|---|---|---|---|---|---|
| pT181/T181 | 0.52 | <0.001 | <0.001 | 0.32 | 0.46 | <0.001 | <0.001 | 0.22 |
| | [0.39, 0.64] | | | | [0.30, 0.63] | | | |
| pT205/T205 | 0.2 | <0.001 | 0.088 | 0.45 | 0.62 | <0.001 | 0.074 | 0.37 |
| | [0.51, ⁶0.74] | | | | [0.47, 0.76] | | | |
| pT217/T217 | 0.69 | <0.001 | Ref. | 0.53 | 0.71 | <0.001 | Ref. | 0.50 |
| | [0.59 0.801 | | | | [0.57, 0.84] | | | |
| O231/T231 | 0.62 | <0.001 | 0.035 | 0.45 | 0.53 | <0.001 | <0.001 | 0.29 |
| | [0.51, 0.74] | | | | [0.37, 0.68] | | | |
| MTBR-tau243 | 0.46 | <0.001 | <0.001 | 0.25 | 0.46 | <0.001 | 6001 | 0.21 |
| | [0.32, 0.59] | | | | [0.29, 0.63] | | | |

Tau-PET
All participants

| | | | | | | | | |
|---|---|---|---|---|---|---|---|---|
| p1181/T181 | 0.65 | <0.001 | <0.001 | 0.43 | 0.40 | <0.001 | <0.001 | 0.16 |
| | [0.58, 0.72] | | | | [0.26, 0.54] | | | |
| pT205/T205 | 0.76 | <0.001 | <0.001 | 0.58 | 0.69 | <0.001 | 0.085 | 0.45 |
| | [0.70, 0.82] | | | | [0.57, 0.80] | | | |
| p1217/T217 | 0.77 | <0.001 | <0.001 | 0.59 | 0.61 | <0.001 | <0.001 | 0.35 |
| | [0.71, 0.83] | | | | [0.49, 0.73] | | | |
| pT231/T231 | 0.60 | <0.001 | <0.001 | 0.36 | 0.45 | <0.001 | <0.001 | 0.19 |
| | [0.52, 0.68] | | | | [0.31, 0.59] | | | |
| MTBR-tau243 | 0.85 | <0.001 | Ref. | 0.69 | 0.76 | <0.001 | Ref. | 0.51 |
| | [0.80, 0.90] | | | | [0.65, 0.87] | | | |

Aβ-positive participants

| | | | | | | | | |
|---|---|---|---|---|---|---|---|---|
| pT181/11 81 | 0.58 | <0.001 | <0.001 | 0.34 | 0.25 | 0.007 | <0.001 | 0.05 |
| | [0.49, 0.68] | | | | [0.07, 0.43] | | | |
| pT205/T205 | 0.74 | <0.001 | 0.001 | 0.55 | 0.67 | <0.001 | 0.092 | 0.42 |
| | [0.67, 0.82] | | | | [0.52, 0.81] | | | |
| p1217/T217 | 0.76 | <0.001 | 0.001 | 0.58 | 0.58 | <0.001 | 0.001 | 0.32 |
| | [0.69, 0.84] | | | | [0.43, 0.73] | | | |
| p123111231 | 0.51 | <0.001 | <0.001 | 0.27 | 0.31 | 0.001 | <0.001 | 0.08 |
| | [0.41, 0.61] | | | | [0.13, 0.48] | | | |
| MTBR-tau243 | 0.84 | <0.001 | Ref. | 0.67 | 0.76 | <0.001 | Ref. | 0.53 |
| | [0.77, 0.91] | | | | [0.63, 0.89] | | | |

TABLE 5
Proportion of variation of CSF biomarker levels explained by amyloid and tau

| | BioFINDER-2 | | | | | | | Knight ADRC | | | | | | |
|---|---|---|---|---|---|---|---|---|---|---|---|---|---|---|
| | | | | ppR² | | | | | | | ppR² | | | |
| | pR² | ppR² | pR² | Tau | | | | pR² | ppR² | pR² | Tau | | | |
| Biomarker | Aβ | Aβ (%) | Tau | (%) | R² | ΔR² | p | Aβ | Aβ (%) | Tau | (%) | R² | ΔR² | p |

Amyloid-PET

| | | | | | | | | | | | | | | |
|---|---|---|---|---|---|---|---|---|---|---|---|---|---|---|
| pT181/T181 | 0.32 | 55.7% | 0.13 | 22.0% | 0.58 | −0.19 | 0.006 | 0.25 | 70.1% | 0.02 | 4.9% | 0.36 | −0.24 | 0.003 |
| pT205/T205 | 0.29 | 45.5% | 0.25 | 39.7% | 0.64 | −0.04 | 0.657 | 0.27 | 45.2% | 0.27 | 45.4% | 0.59 | 0.00 | 0.990 |
| pT217/T217 | 0.57 | 74.7% | 0.19 | 24.7% | 0.77 | −0.38 | <0.001 | 0.51 | 75.1% | 0.14 | 19.9% | 0.68 | −0.38 | <0.001 |
| pT231/T231 | 0.46 | 76.0% | 0.02 | 3.3% | 0.6 | −0.44 | <0.001 | 0.31 | 68.8% | 0.04 | 8.6% | 0.45 | −0.27 | <0.001 |
| MTBR-tau243 | 0.14 | 22.3% | 0.38 | 60.6% | 0.63 | 0.24 | <0.001 | 0.09 | 16.0% | 0.36 | 66.7% | 0.54 | 0.27 | <0.001 |

CSF Aβ42/40

| | | | | | | | | | | | | | | |
|---|---|---|---|---|---|---|---|---|---|---|---|---|---|---|
| pT181/T181 | 0.33 | 52.9% | 0.21 | 33.7% | 0.62 | −0.12 | 0.014 | 0.41 | 82.8% | 0.02 | 3.5% | 0.5 | −0.39 | <0.001 |
| pT205/T205 | 0.13 | 19.6% | 0.43 | 66.8% | 0.64 | 0.3 | <0.001 | 0.20 | 36.5% | 0.32 | 57.6% | 0.56 | 0.12 | 0.125 |
| pT217/T217 | 0.54 | 66.6% | 0.46 | 56.5% | 0.82 | −0.08 | 0.044 | 0.71 | 87.6% | 0.23 | 28.0% | 0.81 | −0.48 | <0.001 |
| pT231/T231 | 0.46 | 69.8% | 0.13 | 19.1% | 0.67 | −0.34 | <0.001 | 0.51 | 84.0% | 0.04 | 7.0% | 0.61 | −0.47 | <0.001 |
| MTBR-tau243 | 0.16 | 21.4% | 0.55 | 75.1% | 0.73 | 0.39 | <0.001 | 0.20 | 33.5% | 0.38 | 63.8% | 0.59 | 0.18 | 0.014 |

TABLE 6
Actual p-values of the differences in CSF biomarker levels

| | | | | |
|---|---|---|---|---|
| Cohort | Predictor | pR² | R² | AICc |

All participants
Aβ-PET

| | | | | |
|---|---|---|---|---|
| BioFINDER-2 | pT205/T205 | 0.06 | 0.77 | 370.2 |
| | pT217/T217 | 0.16 | | |
| | Aβ42/40 | 0.11 | | |
| Knight ADRC | pT205/7205 | 0.02 | 0.73 | 261.7 |
| | pT217fT217 | 0.17 | | |
| | Aβ42/40 | 0.03 | | |

Tau-PET - Braak I-IV region

| | | | | |
|---|---|---|---|---|
| BioFINDER-2 | pT205/T205 | 0.22 | 0.75 | 614.1 |
| | MTBR-tau243 | 0.40 | | |
| Knight ADRC | pT205/T205 | 0.14 | 0.58 | 339.5 |
| | MTBR-tau243 | 0.24 | | |

MMSE

| | | | | |
|---|---|---|---|---|
| BioFINDER-2 | pT205/T205 | 0.1 | 0.48 | 754.6 |
| | MTBR-tau243 | 0.12 | | |
| Knight ADRC | pT205/T205 | 0.05 | 0.34 | 415.2 |
| | MTBR-tau243 | 0.11 | | |

Aβ-positive participants

| | | | | |
|---|---|---|---|---|
| BioFINDER-2 | pT205/T205 | 0.09 | 0.61 | 341 |
| | pT217/T217 | 0.03 | | |
| | pT231/T231 | 0.02 | | |
| | Aβ42/40 | 0.01 | | |
| | Age | 0.03 | | |
| Knight ADRC | pT205/T205 | 0.04 | 0.51 | 258.5 |
| | pT217/T217 | 0.25 | | |

Tau-PET - Braak I-IV region

| | | | | |
|---|---|---|---|---|
| BioFINDER-2 | pT205/T205 | 0.23 | 0.74 | 433.9 |
| | MTBR-tau243 | 0.41 | | |
| Knight ADRC | pT205/T205 | 0.14 | 0.58 | 239.9 |
| | MTBR-tau243 | 0.28 | | |

MMSE

| | | | | |
|---|---|---|---|---|
| BioFINDER-2 | pT205/T205 | 0.09 | 0.41 | 609.9 |
| | MTBR-tau243 | 0.12 | | |
| Knight ADRC | pT205/T205 | 0.04 | 0.36 | 291.1 |
| | MTBR-tau243 | 0.13 | | |
| | Education | 0.06 | | |

Table 7 shows actual p-values of the differences in CSF biomarker levels (BioFINDER-2, n=448) by diagnostic groups were tested using ANCOVA adjusted for age and sex. Post-hoc analyses were performed two-sided using the Tuckey test. Amyloid-positive participants were selected based on CSF Aβ42/40 (CSF Aβ42/40<0.08). Abbreviations: AD+, Alzheimer's disease dementia amyloid positive; CBS, corticobasal syndrome; CU−, cognitively unimpaired amyloid negative; CU+, cognitively unimpaired amyloid positive; FTD, frontotemporal dementia; MCI+, mild cognitive impairment amyloid positive; MTBR, microtubule binding region; PD, Parkinson's disease; PDD, Parkinson's disease dementia; PPA, primary progressive aphasia; PSP, progressive supranuclear palsy.

TABLE 7
CSF biomarkers by diagnosis

| Biomarker | pT181/T181 | pT205/T205 | p217/T217 | pT231/T231 | MTBR-tau243 |
|---|---|---|---|---|---|
| CU+ vs. CU− | <0.001 | 0.002 | <0.001 | <0.001 | 0.466 |
| MCI+ vs. CU− | <0.001 | <0.001 | <0.001 | <0.001 | <0.001 |
| ADD+ vs. CU− | <0.001 | <0.001 | <0.001 | <0.001 | <0.001 |
| PSP/CBS vs. CU− | 0.560 | 0.431 | 0.354 | 0.402 | 1 |
| FTD/PPA vs. CU− | 0.440 | 0.988 | 0.918 | 0.999 | 0.980 |
| PD/PDD vs. CU− | 1 | 0.964 | 0.960 | 0.737 | 1 |
| MCI+ vs. CU+ | 0.001 | <0.001 | <0.001 | 0.015 | 0.003 |
| ADD+ vs. CU+ | <0.001 | <0.001 | <0.001 | <0.001 | <0.001 |
| PSP/CBS vs. CU+ | <0.001 | 0.968 | 0.003 | <0.001 | 0.860 |
| FTD/PPA vs. CU+ | <0.001 | 0.25 | <0.001 | <0.001 | 0.995 |
| PD/PDD vs. CU+ | <0.001 | 0.428 | <0.001 | <0.001 | 0.668 |
| ADD+ vs. MCI+ | <0.001 | <0.001 | <0.001 | <0.001 | <0.001 |
| PSP/CBS vs. MCI+ | <0.001 | <0.001 | <0.001 | <0.001 | 0.001 |
| FTD/PPA vs. MCI+ | <0.001 | <0.001 | <0.001 | <0.001 | 0.006 |
| PD/PDD vs. MCI+ | <0.001 | <0.001 | <0.001 | <0.001 | <0.001 |
| PSP/CBS vs. ADD+ | <0.001 | <0.001 | <0.001 | <0.001 | <0.001 |
| FTD/PPA vs. ADD+ | <0.001 | <0.001 | <0.001 | <0.001 | <0.001 |
| PD/PDD vs. ADD+ | <0.001 | <0.001 | <0.001 | <0.001 | <0.001 |
| FTD/PPA vs. PSP/CBS | 1 | 0.935 | 0.974 | 0.825 | 0.998 |
| PD/PDD vs. PSP/CBS | 0.780 | 0.977 | 0.963 | 0.999 | 1 |
| PD/PDD vs. FTD/PPA | 0.706 | 1 | 1 | 0.971 | 0.979 |

Abbreviations:
Aβ, amyloid;
CI, confidence interval;
CSF, cerebrospinal fluid;
MTBR, microtubule binding region

Table 8 shows linear regression models were used to assess the associations between CSF biomarkers and CSF Aβ42/40 adjusting for age and sex (BioFINDER-2: n=427, Knight ADRC: n=219; except for pT231/T231 in which n=184). P comparison (p comp.) was calculated to assess differences between the strongest association (Ref.) and each of the other CSF biomarkers using bootstrapping (n=500) from adjusted β. Significant p comparison (<0.05) suggests weaker associations. Association p-values were based on two-sided tests and bootstrapping p-values from one-sided tests, all unadjusted for multiple comparisons.

TABLE 8
Associations between CSF biomarkers and CSF Aβ42/40

| | BioFINDER-2 | | | | Knight ADRC | | | |
|---|---|---|---|---|---|---|---|---|
| | β std | | | | β std | | | |
| Biomarker | [95% CI] | p (β) | p (comp.) | R² | [95% CI] | p (β) | p (comp.) | R² |
| All participants | | | | | | | | |
| pT181/T181 | −0.70 | <0.001 | <0.001 | 0.52 | −0.69 | <0.001 | <0.001 | 0.53 |
| | [−0.77, −0.63] | | | | [−0.79, −0.58] | | | |
| pT205/T205 | −0.59 | <0.001 | <0.001 | 0.38 | −0.59 | <0.001 | <0.001 | 0.41 |
| | [−0.67, −0.52] | | | | [−0.71, −0.48] | | | |
| pT217/T217 | −0.8 | <0.001 | Ref. | 0.66 | −0.88 | <0.001 | Ref. | 0.79 |
| | [−0.86, −0.74] | | | | [−0.95, −0.81] | | | |
| pT231/T231 | −0.77 | <0.001 | 0.026 | 0.62 | −0.72 | <0.001 | 0.001 | 0.61 |
| | [−0.83, −0.71] | | | | [−0.81, −0.63] | | | |
| MTBR-tau243 | −0.63 | <0.001 | <0.001 | 0.41 | −0.59 | <0.001 | <0.001 | 0.39 |
| | [−0.70, −0.55] | | | | [−0.71, −0.47] | | | |

Abbreviations: Aβ, amyloid; CI, confidence interval; CSF, cerebrospinal fluid; MTBR, microtubule binding region.

Table 9 shows linear regression models were used to assess the associations between CSF biomarkers and tau-PET in different Braak regions (SUVR) adjusting for age and sex (BioFINDER-2: n=443, Knight ADRC: n=219; except for pT231/T231 in which n=184). Amyloid-positive participants (BioFINDER-2: n=287, Knight ADRC: n=136; except for pT231/T231 in which n=117) were selected based on CSF Aβ42/40 previously validated cut-offs (CSF Aβ42/40<0.08 in BioFINDER-2 and CSF Aβ42/40<0.0673 in Knight ADRC). P comparison (p comp.) was calculated to assess differences between the strongest association (Ref.) and each of the other CSF biomarkers using bootstrapping (n=500) from adjusted B. Significant p comparison (<0.05) suggests weaker associations. Association p-values were based on two-sided tests and bootstrapping p-values from one-sided tests, all unadjusted for multiple comparisons.

TABLE 9
Associations between CSF biomarkers and tau-PET SUVR in different Braak regions

| | All participants | | | | | | | |
|---|---|---|---|---|---|---|---|---|
| | BioFINDER-2 | | | | Knight ADRC | | | |
| | β std | p | p | | β std | p | p | |
| Biomarker | [95% CI] | (β) | (comp.) | R² | [95% CI] | (β) | (comp.) | R² |

Braak I-II

| | | | | | | | | |
|---|---|---|---|---|---|---|---|---|
| pT181/ | 0.68 | <0.001 | <0.001 | 0.48 | 0.43 | <0.001 | <0.001 | 0.23 |
| T181 | [0.61, 0.75] | | | | [0.30, 0.56] | | | |
| pT205/ | 0.77 | <0.001 | 0.001 | 0.60 | 0.62 | <0.001 | 0.474 | 0.40 |
| T205 | [0.71, 0.83] | | | | [0.50, 0.74] | | | |
| pT217/ | 0.81 | <0.001 | 0.007 | 0.65 | 0.58 | <0.001 | 0.119 | 0.36 |
| T217 | [0.75, 0.86] | | | | [0.46, 0.70] | | | |
| pT231/ | 0.64 | <0.001 | <0.001 | 0.42 | 0.43 | <0.001 | <0.001 | 0.21 |
| T231 | [0.57, 0.71] | | | | [0.29, 0.57] | | | |
| MTBR- | 0.85 | <0.001 | Ref. | 0.70 | 0.65 | <0.001 | Ref. | 0.42 |
| tau243 | [0.80, 0.91] | | | | [0.53, 0.77] | | | |

Braak III-IV

| | | | | | | | | |
|---|---|---|---|---|---|---|---|---|
| pT181/ | 0.65 | <0.001 | <0.001 | 0.42 | 0.28 | <0.001 | <0.001 | 0.09 |
| T181 | [0.57, 0.72] | | | | [0.13, 0.42] | | | |
| pT205/ | 0.76 | <0.001 | <0.001 | 0.57 | 0.56 | <0.001 | 0.470 | 0.30 |
| T205 | [0.70, 0.82] | | | | [0.43, 0.69] | | | |
| pT217/ | 0.77 | <0.001 | <0.001 | 0.58 | 0.44 | <0.001 | 0.001 | 0.19 |
| T217 | [0.70, 0.83] | | | | [0.30, 0.58] | | | |
| pT231/ | 0.59 | <0.001 | <0.001 | 0.36 | 0.32 | <0.001 | <0.001 | 0.11 |
| T231 | [0.52, 0.67] | | | | [0.17, 0.47] | | | |
| MTBR- | 0.84 | <0.001 | Ref. | 0.68 | 0.59 | <0.001 | Ref. | 0.32 |
| tau243 | [0.79, 0.90] | | | | [0.46, 0.72] | | | |

Braak V-VI

| pT181/ | 0.57 | <0.001 | <0.001 | 0.32 | 0.36 | <0.001 | <0.001 | 0.13 |
|---|---|---|---|---|---|---|---|---|
| T181 | [0.49, 0.65] | | | | [0.21, 0.50] | | | |
| pT205/ | 0.69 | <0.001 | 0.013 | 0.46 | 0.65 | <0.001 | 0.095 | 0.40 |
| T205 | [0.62, 0.76] | | | | [0.54, 0.77] | | | |
| pT217/ | 0.67 | <0.001 | <0.001 | 0.44 | 0.56 | <0.001 | <0.001 | 0.30 |
| T217 | [0.60, 0.75] | | | | [0.44, 0.69] | | | |
| pT231/ | 0.52 | <0.001 | <0.001 | 0.27 | 0.40 | <0.001 | <0.001 | 0.15 |
| T231 | [0.44, 0.6] | | | | [0.25, 0.54] | | | |
| MTBR- | 0.76 | <0.001 | Ref. | 0.54 | 0.73 | <0.001 | Ref. | 0.47 |
| tau243 | [0.69, 0.82] | | | | [0.61, 0.84] | | | |
| | Amyloid-positive participants | | | | | | | |
| | BioFINDER-2 | | | | Knight ADRC | | | |
| | β std | p | p | | β std | p | p | |
| Biomarker | [95% CI] | (β) | (comp.) | R² | [95% CI] | (β) | (comp.) | R² |

Braak I-II

| | | | | | | | | |
|---|---|---|---|---|---|---|---|---|
| pT181/ | 0.63 | <0.001 | <0.001 | 0.40 | 0.26 | 0.005 | <0.001 | 0.07 |
| T181 | [0.53, 0.72] | | | | [0.08, 0.44] | | | |
| pT205/ | 0.76 | <0.001 | 0.018 | 0.58 | 0.58 | <0.001 | 0.887 | 0.33 |
| T205 | [0.69, 0.84] | | | | [0.43, 0.74] | | | |
| pT217/ | 0.78 | <0.001 | 0.039 | 0.62 | 0.49 | <0.001 | 0.093 | 0.24 |
| T217 | [0.71, 0.86] | | | | [0.33, 0.65] | | | |
| pT231/ | 0.54 | <0.001 | <0.001 | 0.30 | 0.25 | 0.005 | <0.001 | 0.07 |
| T231 | [0.44, 0.64] | | | | [0.08, 0.43] | | | |
| MTBR- | 0.83 | <0.001 | Ref. | 0.66 | 0.59 | <0.001 | Ref. | 0.34 |
| tau243 | [0.76, 0.9] | | | | [0.44, 0.75] | | | |

Braak III-IV

| | | | | | | | | |
|---|---|---|---|---|---|---|---|---|
| pT181/ | 0.57 | <0.001 | <0.001 | 0.33 | 0.19 | 0.043 | <0.001 | 0.01 |
| T181 | [0.48, 0.67] | | | | [0.01, 0.37] | | | |
| pT205/ | 0.74 | <0.001 | 0.001 | 0.55 | 0.60 | <0.001 | 0.47 | 0.33 |
| T205 | [0.66, 0.82] | | | | [0.45, 0.761 | | | |
| pT217/ | 0.76 | <0.001 | 0.001 | 0.57 | 0.45 | <0.001 | 0.004 | 0.18 |
| T217 | [0.68, 0.83] | | | | [0.28, 0.62] | | | |
| pT231/ | 0.51 | <0.001 | <0.001 | 0.26 | 0.20 | 0.034 | <0.001 | 0.02 |
| T231 | [0.41, 0.61] | | | | [0.01, 0.38] | | | |
| MTBR- | 0.84 | <0.001 | Ref. | 0.67 | 0.64 | <0.001 | Ref. | 0.37 |
| tau243 | [0.77, 0.90] | | | | [0.49, 0.79] | | | |

Braak V-VI

| | | | | | | | | |
|---|---|---|---|---|---|---|---|---|
| pT181/ | 0.52 | <0.001 | <0.001 | 0.28 | 0.21 | 0.022 | <0.001 | 0.02 |
| T181 | [0.42, 0.62] | | | | [0.03, 0.40] | | | |
| pT205/ | 0.68 | <0.001 | 0.009 | 0.47 | 0.64 | <0.001 | 0.085 | 0.37 |
| T205 | [0.60, 0.77] | | | | [0.49, 0.79] | | | |
| pT217/ | 0.68 | <0.001 | <0.001 | 0.47 | 0.53 | <0.001 | <0.001 | 0.26 |
| T217 | [0.60, 0.771 | | | | [0.37, 0.69] | | | |
| pT231/ | 0.47 | <0.001 | <0.001 | 0.23 | 0.26 | 0.004 | <0.001 | 0.05 |
| T231 | [0.37, 0.57] | | | | [0.09, 0.44] | | | |
| MTBR- | 0.76 | <0.001 | Ref. | 0.56 | 0.73 | <0.001 | Ref. | 0.47 |
| tau243 | [0.68, 0.84] | | | | [0.59, 0.86] | | | |

Abbreviations: Aβ, amyloid; CI, confidence interval; CSF, cerebrospinal fluid; MTBR, microtubule binding region; PET, positron emission tomography.

TABLE 10
Characteristics of BioFINDER-2 participants with longitudinal CSF available

| Overall | | CU− | | CU+ | | MCI+ | | AD + | | Non-AD | |
|---|---|---|---|---|---|---|---|---|---|---|---|
| n= | 220 | n= | 79 | n= | 49 | n= | 44 | n= | 29 | n= | 19 |

Demographics

| | | | | | | | | | | | | |
|---|---|---|---|---|---|---|---|---|---|---|---|---|
| Age, years | 220 | 73.0 | 79 | 72.0 | 49 | 74.3 | 44 | 73.1 | 29 | 75.7 | 19 | 69.9 |
| | | (8.74) | | (9.48) | | (7.72) | | (9.27) | | (7.73) | | (7.25) |
| Women, n (%) | 220 | 107 | 79 | 39 | 49 | 23 | 44 | 23 | 29 | 16 | 19 | 6 |
| | | (48.6%) | | (49.4%) | | (46.9%) | | (52.3%) | | (55.2%) | | (31.6%) |
| APOE-e4 carriers, | 220 | 121 | 79 | 26 | 49 | 36 | 44 | 33 | 29 | 21 | 19 | 5 |
| n (%) | | (55.0%) | | (32.9%) | | (73.5%) | | (75.0%) | | (72.4%) | | (26.3%) |
| Years of | 220 | 12.1 | 79 | 11.9 | 49 | 12.0 | 44 | 12.8 | 29 | 11.4 | 19 | 13.0 |
| education | | (3.94) | | (3.21) | | (3.52) | | (5.21) | | (3.83) | | (4.55) |

Amyloid- and tau-status

| | | | | | | | | | | | | |
|---|---|---|---|---|---|---|---|---|---|---|---|---|
| AT status, n (%) | 220 | | 79 | | 49 | | 44 | | 29 | | 19 | |
| A−T− | | 93 | | 79 | | 0 | | 0 | | 0 | | 14 |
| | | (42.3%) | | (100%) | | (0%) | | (0%) | | (0%) | | (73.7%) |
| A+T− | | 66 | | 0 | | 40 | | 21 | | 0 | | 5 |
| | | (30.0%) | | (0%) | | (81.6%) | | (47.7%) | | (0%) | | (26.3%) |
| A+T+ | | 61 | | 0 | | 9 | | 23 | | 29 | | 0 |
| | | (27.7%) | | (0%) | | (18.4%) | | (52.3%) | | (100%) | | (0%) |

CSF tau by mass spectrometry

| | | | | | | | | | | | | |
|---|---|---|---|---|---|---|---|---|---|---|---|---|
| pT181/T181 (%) | 220 | 0.09 | 79 | −0.05 | 49 | 0.42 | 44 | 0.24 | 29 | −0.36 | 19 | 0.17 |
| | | (1.00) | | (0.73) | | (1.09) | | (0.79) | | (1.61) | | (0.62) |
| pT205/T205 (%) | 220 | 0.021 | 79 | 0.008 | 49 | 0.024 | 44 | 0.043 | 29 | 0.026 | 19 | 0.015 |
| | | (0.064) | | (0.043) | | (0.058) | | (0.074) | | (0.096) | | (0.059) |
| pT217/T217 (%) | 220 | 0.15 | 79 | 0.02 | 49 | 0.30 | 44 | 0.35 | 29 | −0.01 | 19 | 0.10 |
| | | (0.41) | | (0.22) | | (0.29) | | (0.47) | | (0.70) | | (0.17) |
| pT231/T231 (%) | 218 | 0.18 | 79 | −0.02 | 49 | 0.38 | 44 | 0.50 | 28 | −0.04 | 18 | 0.03 |
| | | (1.10) | | (0.79) | | (0.92) | | (1.19) | | (1.82) | | (0.77) |
| MTBR-tau243 | 220 | 0.035 | 79 | 0.003 | 49 | 0.017 | 44 | 0.067 | 29 | 0.125 | 19 | 0.002 |
| (pg/ml) | | (0.073) | | (0.012) | | (0.026) | | (0.091) | | (0.116) | | (0.008) |

Abbreviations: AD+, Alzheimer's disease dementia amyloid positive; CSF, cerebrospinal fluid; CU−, cognitively unimpaired amyloid negative; CU+, cognitively unimpaired amyloid positive; MCI+, mild cognitive impairment amyloid positive; MTBR, microtubule binding region; non-AD, non-Alzheimer's disease dementia.
Parenthesis in rows: standard deviation

Longitudinal CSF data was only available in BioFINDER-2. Amyloid-positive participants were selected based on CSF Aβ42/40 previously validated cut-offs (CSF Aβ42/40<0.08 in BioFINDER-2). Tau positivity was assessed based on tau-PET SUVR in the meta-ROI (SUVR>1.32 in both BioFINDER-2). Amyloid (A) and tau (T) status are shown in Table 10.

Table 11 shows differences in CSF longitudinal rates of change by baseline Amyloid (A) and Tau (T) status was assessed using were tested using a Kruskal-Wallis tests and pairwise Wilcoxon tests for post-hoc comparisons. P-values come from two-sided tests uncorrected for multiple comparisons. Cohen's d among different groups were calculated from individual participant slopes from linear regression models. Amyloid-positive participants were selected based on CSF Aβ42/40 previously validated cut-offs (CSF Aβ42/40<0.08 in BioFINDER-2, n=220, except for pT231/T231 in which n=218) or amyloid-PET (SUVR>1.03, n=174). Tau positivity was assessed based on tau-PET SUVR in the Braak I-IV ROI (SUVR>1.32). The greatest rate of change per A/T group is shown in bold.

TABLE 11
Longitudinal CSF biomarkers change by baseline AT status

| | A−T− vs A+T− | | A−T− vs A+T+ | | A+T− vs A+T+ | |
|---|---|---|---|---|---|---|
| Biomarker | Cohen's d | p | Cohen's d | p | Cohen's d | p |

CSF Aβ42/40

| | | | | | | |
|---|---|---|---|---|---|---|
| pT181/T181 | 0.66 | <0.001 | −0.22 | 0.011 | −0.69 | <0.001 |
| pT205/T205 | 0.38 | 0.011 | 0.44 | 0.014 | 0.08 | 0.788 |
| pT217/T217 | 1.39 | <0.001 | 0.21 | 0.617 | −0.56 | <0.001 |
| pT231/T231 | 0.71 | <0.001 | 0.02 | 0.98 | −0.48 | 0.007 |
| MTBR-tau243 | 0.78 | <0.001 | 1.48 | <0.001 | 1.13 | <0.001 |

Amyloid-PET

| | | | | | | |
|---|---|---|---|---|---|---|
| pT181/T181 | 0.78 | <0.001 | −0.17 | 0.277 | −0.8 | 0.001 |
| pT205/T205 | 0.32 | 0.024 | 0.42 | 0.041 | 0.06 | 0.738 |
| pT217/T217 | 1.34 | <0.001 | 0.42 | 0.178 | −0.54 | 0.011 |
| pT231/T231 | 0.82 | <0.001 | 0.14 | 0.368 | −0.58 | 0.023 |
| MTBR-tau243 | 0.78 | <0.001 | 1.72 | <0.001 | 1.12 | <0.001 |

TABLE 12
Associations between AD-biomarkers and MMSE

| | BioFINDER-2 | | | | Knight ADRC | | | |
|---|---|---|---|---|---|---|---|---|
| Biomarker | β std[95% CI] | p (β) | p (comp.) | R² | β std[95% CI] | p (β) | p (comp.) | R² |

All participants

| | | | | | | | | |
|---|---|---|---|---|---|---|---|---|
| pT181/T181 | −0.44 | <0.001 | <0.001 | 0.21 | −0.24 | 0.001 | <0.001 | 0.15 |
| | [−0.54, −0.35] | | | | [−0.38, −0.10] | | | |
| pT205/T205 | −0.63 | <0.001 | 0.328 | 0.40 | −0.48 | <0.001 | 0.262 | 0.30 |
| | [−0.72, −0.55] | | | | [−0.6, −0.35] | | | |
| pT217/T217 | −0.6 | <0.001 | 0.001 | 0.37 | −0.4 | <0.001 | 0.003 | 0.24 |
| | [−0.69, −0.52] | | | | [−0.53, −0.26] | | | |
| pT231/T231 | −0.47 | <0.001 | <0.001 | 0.23 | −0.26 | 0.001 | <0.001 | 0.16 |
| | [−0.56, −0.37] | | | | [−0.41, −0.12] | | | |
| MTBR-tau243 | −0.65 | <0.001 | Ref. | 0.41 | −0.54 | <0.001 | Ref. | 0.35 |
| | [−0.74, −0.57] | | | | [−0.67, −0.42] | | | |
| Aβ42/40 | 0.43 | <0.001 | <0.001 | 0.20 | 0.28 | <0.001 | 0.067 | 0.17 |
| | [0.34, 0.53] | | | | [0.13, 0.42] | | | |
| Amyloid-PET | −0.2 | <0.001 | <0.001 | 0.22 | −0.34 | <0.001 | 0.001 | 0.21 |
| | [−0.25, −0.15] | | | | [−0.48, −0.21] | | | |
| Tau-PET | −0.72 | <0.001 | 0.036 | 0.52 | −0.64 | <0.001 | <0.001 | 0.50 |
| | [−0.79, −0.64] | | | | [−0.74, −0.53] | | | |

Aβ-positive participants

| | | | | | | | | |
|---|---|---|---|---|---|---|---|---|
| pT181/T181 | −0.28 | <0.001 | <0.001 | 0.09 | −0.11 | 0.208 | <0.001 | 0.10 |
| | [−0.39, −0.17] | | | | [−0.29, 0.06] | | | |
| pT205/T205 | −0.53 | <0.001 | 0.510 | 0.29 | −0.44 | <0.001 | 0.222 | 0.28 |
| | [−0.63, −0.43] | | | | [−0.60, −0.28] | | | |
| pT217/T217 | −0.48 | <0.001 | 0.002 | 0.24 | −0.36 | <0.001 | 0.001 | 0.21 |
| | [−0.59, −0.38] | | | | [−0.52, −0.19] | | | |
| pT231/T231 | −0.31 | <0.001 | <0.001 | 0.11 | −0.14 | 0.113 | <0.001 | 0.12 |
| | [−0.42, −0.20] | | | | [−0.31, 0.03] | | | |
| MTBR-tau243 | −0.56 | <0.001 | Ref. | 0.31 | −0.54 | <0.001 | Ref. | 0.37 |
| | [−0.66, −0.46] | | | | [−0.69, −0.39] | | | |
| Aβ42/40 | 0.23 | <0.001 | <0.001 | 0.06 | 0.17 | 0.053 | 0.139 | 0.12 |
| | [0.11, 0.34] | | | | [0.00, 0.35] | | | |
| Amyloid-PET | −0.14 | <0.001 | <0.001 | 0.09 | −0.33 | <0.001 | 0.003 | 0.19 |
| | [−0.21, −0.07] | | | | [−0.49, −0.16] | | | |
| Tau-PET | −0.63 | <0.001 | 0.052 | 0.40 | −0.63 | <0.001 | <0.001 | 0.50 |
| | [−0.72, −0.54] | | | | [−0.76, −0.50] | | | |

Abbreviations: Aβ, amyloid; CI, confidence interval; CSF, cerebrospinal fluid; MMSE, Mini-Mental State Examination; MTBR, microtubule binding region; PET, positron emission tomography.

Table 12 shows linear regression models were used to assess the associations between CSF biomarkers and MMSE for BioFINDER-2 and Knight ADRC participants adjusting for age, sex and education (BioFINDER-2: n=342, Knight ADRC: n=219, except for pT231/T231 in which n=184). Amyloid-positive participants (BioFINDER-2: n=261, Knight ADRC: n=136, except for pT231/T231 in which n=117) were selected based on CSF Aβ42/40 previously validated cut-offs (CSF Aβ42/40<0.08 in BioFINDER-2 and CSF Aβ42/40<0.0673 in Knight ADRC). P comparison (p comp.) was calculated to assess differences between the strongest association of CSF tau markers (Ref.) and each of the other biomarkers using bootstrapping (n=500) from adjusted β. Significant p comparison (<0.05) suggests weaker associations, except in the case of Tau-PET. Non-AD cases were excluded from BioFINDER-2 cohort for this analysis. Association p-values were based on two-sided tests and bootstrapping p-values from one-sided tests, all unadjusted for multiple comparisons.

TABLE 13
Predicting AD-related continuous measure by CSF biomarkers

| | BioFINDER-2 | | Knight ADRC | | | BioFINDER-2 | | Knight ADRC | | | BioFINDER-2 | | Knight ADRC | |
|---|---|---|---|---|---|---|---|---|---|---|---|---|---|---|
| Model | R² | AICc | R² | AICc | Model | R² | AICc | R² | AICc | Model | R² | AICc | R² | AICc |

| | | |
|---|---|---|
| Amyloid-PET (CL) | Tau-PET (SUVR) | MMSE |

All participants

| | | | | | | | | | | | | | | |
|---|---|---|---|---|---|---|---|---|---|---|---|---|---|---|
| Base | 0.03 | 742.2 | 0.03 | 480.0 | Base | 0.01 | 1196.4 | 0.01 | 483.1 | Base | 0.02 | 970.3 | 0.10 | 468.5 |
| pT181/T181 | 0.53 | 549.5 | 0.43 | 387.9 | pT181/T181 | 0.43 | 961.8 | 0.15 | 456.1 | pT181/T181 | 0.21 | 896.1 | 0.07 | 471.3 |
| pT205/T205*† | 0.51 | 563.1 | 0.46 | 381.0 | pT205/T205*† | 0.59 | 827.3 | 0.44 | 384.5 | pT205/T205*† | 0.41 | 797.0 | 0.26 | 433.0 |
| pT217/T217*† | 0.73 | 404.5 | 0.73 | 265.2 | pT217/T217 | 0.60 | 813.1 | 0.36 | 409.6 | pT217/T217 | 0.37 | 817.6 | 0.18 | 449.8 |
| pT231/T231 | 0.61 | 503.5 | 0.45 | 381.7 | pT231/T231 | 0.38 | 1002.1 | 0.20 | 447.8 | pT231/T231 | 0.23 | 886.9 | 0.08 | 469.7 |
| MTBR-tau243 | 0.42 | 605 | 0.34 | 412.7 | MTBR- | 0.68 | 715.6 | 0.51 | 363.2 | MTBR- | 0.42 | 790.3 | 0.30 | 423.0 |
| | | | | | tau243*† | | | | | tau243*† | | | | |
| Aβ42/40*† | 0.59 | 514.8 | 0.61 | 325.2 | Aβ42/40 | 0.30 | 1051.8 | 0.19 | 447.6 | Aβ42/40 | 0.20 | 899.0 | 0.08 | 469.7 |
| Parsimonious | 0.77 | 370.2 | 0.73 | 261.7 | Parsimonious | 0.75 | 614.1 | 0.58 | 339.5 | Parsimonious | 0.48 | 754.6 | 0.34 | 415.2 |

| | | | | | | |
|---|---|---|---|---|---|---|
| — | — | Tau-PET | 0.52 | 724.9 | 0.44 | 385.8 |

Amyloid positive participants

| | | | | | | | | | | | | | | |
|---|---|---|---|---|---|---|---|---|---|---|---|---|---|---|
| Base | 0.05 | 495.8 | 0.01 | 341.4 | Base | 0.01 | 817.4 | 0.00 | 343.4 | Base | 0.02 | 742.9 | 0.11 | 330.3 |
| pT181/T181 | 0.35 | 429.0 | 0.21 | 314.4 | pT181/T181 | 0.34 | 698.6 | 0.06 | 334.8 | pT181/T181 | 0.10 | 718.6 | 0.02 | 339.9 |
| pT205/T205*† | 0.43 | 403.9 | 0.34 | 291.9 | pT205/T205*† | 0.56 | 585.7 | 0.42 | 277.6 | pT205/T205*† | 0.33 | 642.1 | 0.23 | 310.0 |
| pT217/T217*† | 0.56 | 359.4 | 0.49 | 262.3 | pT217/T217 | 0.58 | 569.8 | 0.33 | 294.6 | pT217/T217 | 0.28 | 658.0 | 0.15 | 322.5 |
| pT231/T231* | 0.43 | 405.6 | 0.27 | 304.3 | pT231/T231 | 0.26 | 730.3 | 0.09 | 330.7 | pT231/T231 | 0.12 | 711.3 | 0.02 | 339.6 |
| MTBR-tau243 | 0.34 | 431.0 | 0.20 | 315.5 | MTBR- | 0.66 | 506.4 | 0.51 | 256.8 | MTBR- | 0.35 | 633.0 | 0.28 | 302.6 |
| | | | | | tau243*† | | | | | tau243*† | | | | |
| Aβ42/40* | 0.27 | 449.0 | 0.19 | 316.3 | Aβ42/40 | 0.17 | 765.6 | 0.09 | 330.5 | Aβ42/40 | 0.07 | 725.8 | 0.02 | 339.6 |
| Parsimonious | 0.61 | 341.0 | 0.51 | 258.5 | Parsimonious | 0.74 | 433.9 | 0.58 | 239.9 | Parsimonious | 0.41 | 609.9 | 0.36 | 291.1 |

| | | | | | | |
|---|---|---|---|---|---|---|
| — | — | Tau-PET | 0.46 | 587.9 | 0.44 | 273.9 |

Abbreviations: Aβ, amyloid; AICc, corrected Akaike information criterion; CI, confidence interval; CL, Centiloids; CSF, cerebrospinal fluid; LASSO, least absolute shrinkage and selection operator; MMSE, Mini-Mental State Examination; MTBR, microtubule binding region; PET, positron emission tomography.

Table 13 shows linear regression models were used for predicting amyloid-PET (BioFINDER-2: n=256, Knight ADRC: n=184), tau-PET (BioFINDER-2: n=422, Knight ADRC: 184) and MMSE (BioFINDER-2: n=342, Knight ADRC: n=184). Base model included age and sex (and education for MMSE) as predictors. Parsimonious model was obtained with the optimal combination of CSF biomarkers and demographics (age and/or sex and/or education) assessed using a LASSO regression. Biomarkers included in the parsimonious models are depicted with * for BioFINDER-2 and with † for Knight ADRC. The other models used only individual CSF biomarkers as predictors. Tau-PET was used as predictor in an independent model for predicting MMSE as a comparison. Optimal models are shown in bold. Non-AD cases were excluded from BioFINDER-2 cohort for the cognition analyses. Amyloid-positive participants (BioFINDER-2: amyloid-PET: n=172, tau-PET: n=287, MMSE: n=261; Knight ADRC: n=117 for all cases) were selected based on CSF Aβ42/40 previously validated cut-offs (CSF Aβ42/40<0.08 in BioFINDER-2 and CSF Aβ42/40<0.0673 in Knight ADRC).

TABLE 14
Diagnostic accuracies of 4R specific CSF MTBR-tau to distinguish CBD from FTLD-tau and control.

| | N per | | | | | | | |
|---|---|---|---|---|---|---|---|---|
| Groups | group | Test | AUC | 95% CI | P value | Sensitivity (%) | Specificity (%) | Cutoff |
| CBD vs NC | 18 vs 29 | CSF MTBR-tau275/t- | 0.810 | 0.672 to 0.949 | 0.0004 | 93.1 | 72.2 | 0.00582 |
| | | tau | | | | | | |
| | | CSF MTBR-tau282/t- | 0.889 | 0.790 to 0.988 | <0.0001 | 89.7 | 77.9 | 0.01170 |
| | | tau | | | | | | |
| CBD vs | 18 vs 21 | CSF MTBR-tau275/t- | 0.701 | 0.530 to 0.872 | 0.0323 | 81.0 | 66.7 | 0.00559 |
| FTLD-TDP | | tau | | | | | | |
| | | CSF MTBR-tau282/t- | 0.770 | 0.619 to 0.921 | 0.0041 | 81.0 | 72.2 | 0.01096 |
| | | tau | | | | | | |
| CBD vs other | 18 vs 29 | CSF MTBR-tau275/t- | 0.835 | 0.716 to 0.955 | 0.0001 | 89.7 | 66.7 | 0.00563 |
| FTLD-tau | | tau | | | | | | |
| (PSP, AGD, | | CSF MTBR-tau282/t- | 0.855 | 0.747 to 0.963 | <0.0001 | 75.9 | 89.0 | 0.01219 |
| PiD) | | tau | | | | | | |
| CBD vs PiD | 18 vs 5 | CSF MTBR-tau275/t- | 0.800 | 0.572 to 1.000 | 0.0442 | 80.0 | 72.2 | 0.00599 |
| | | tau | | | | | | |
| | | CSF MTBR-tau282/t- | 0.806 | 0.543 to 1.000 | 0.0404 | 80.0 | 89.0 | 0.01219 |
| | | tau | | | | | | |
| CBD vs PSP | 18 vs 22 | CSF MTBR-tau275/t- | 0.828 | 0.696 to 0.960 | 0.0004 | 90.0 | 66.7 | 0.00563 |
| | | tau | | | | | | |
| | | CSF MTBR-tau282/t- | 0.854 | 0.736 to 0.972 | 0.0001 | 72.7 | 89.0 | 0.01220 |
| | | tau | | | | | | |
| CBD vs PSP | 15 vs 17 | CSF MTBR-tau275/t- | 0.859 | 0.718 to 0.999 | 0.0005 | 82.4 | 86.7 | 0.00648 |
| (AD co-pathology | | tau | | | | | | |
| removed) | | CSF MTBR-tau282/t- | 0.886 | 0.763 to 1.000 | 0.0002 | 94.1 | 80.0 | 0.01164 |
| | | tau | | | | | | |

NC: normal control, AD: Alzheimer disease, CBD: corticobasal degeneration, PSP: progressive supranuclear palsy, AGD: argyrophilic grain disease, PiD: Pick's disease, TDP: TAR DNA-binding protein.

TABLE 15
CSF sample demographic for the work to identify the endogenous
MTBR-tau243 species which surrogates tau-PET

| | | | | | MTBR-tau243 | tau | MTBR-tau243 | |
|---|---|---|---|---|---|---|---|---|
| | | | | tau PET (4- | by CX | PET | by CX (ng/mL) | pool |
| # | ID | CDR | p217% | regions) | (ng/mL) | AVG. | AVG. | volume |
| 24 | D62901 | 0 | 1.91 | 1.27 | 2.04 | 1.14 | 2.39 | Total 10 ml |
| 24 | D62901 | 0 | 1.91 | 1.27 | 2.04 | | | |
| 44 | D65780 | 0 | 2.16 | 1.24 | 2.14 | | | |
| 44 | D65780 | 0 | 2.16 | 1.24 | 2.14 | | | |
| 28 | D64752 | 0 | 2.40 | 1.09 | 2.32 | | | |
| 28 | D64752 | 0 | 2.40 | 1.09 | 2.32 | | | |
| 23 | D62703 | 0 | 1.93 | 1.12 | 2.65 | | | |
| 23 | D62703 | 0 | 1.93 | 1.12 | 2.65 | | | |
| 4 | D64142 | 0 | 2.37 | 0.97 | 2.82 | | | |
| 4 | D64142 | 0 | 2.37 | 0.97 | 2.82 | | | |
| 21 | D62061 | 0.5 | 5.53 | 1.51 | 4.42 | 1.72 | 4.94 | Total 10 ml |
| 21 | D62061 | 0.5 | 5.53 | 1.51 | 4.42 | | | |
| 3 | D63054 | 0 | 7.51 | 1.89 | 4.85 | | | |
| 3 | D63054 | 0 | 7.51 | 1.89 | 4.85 | | | |
| 18 | 065028 | 0 | 5.15 | 1.46 | 5.07 | | | |
| 18 | D65028 | 0 | 5.15 | 1.46 | 5.07 | | | |
| 54 | D64432 | 0 | 6.22 | 1.96 | 5.17 | | | |
| 54 | 064432 | 0 | 6.22 | 1.96 | 5.17 | | | |
| 39 | D64543 | 0.5 | 8.64 | 1.80 | 5.20 | | | |
| 39 | D64543 | 0.5 | 8.64 | 1.80 | 5.20 | | | |
| 34 | D63503 | 0 | 7.54 | 1.99 | 6.30 | 2.60 | 11.79 | Total 10 ml |
| 34 | D63503 | 0 | 7.54 | 1.99 | 6.30 | | | |
| 52 | D22574 | 0.5 | 7.18 | 2.00 | 8.07 | | | |
| 52 | D22574 | 0.5 | 7.18 | 2.00 | 8.07 | | | |
| 2 | D84588 | 1 | 5.45 | 2.03 | 8.35 | | | |
| 2 | D84588 | 1 | 5.45 | 2.03 | 8.35 | | | |
| 31 | D22235 | 2 | 7.70 | 3.94 | 16.70 | | | |
| 31 | D22235 | 2 | 7.70 | 3.94 | 16.70 | | | |
| 19 | D65927 | 0.5 | 8.47 | 3.04 | 19.52 | | | |
| 19 | D65927 | 0.5 | 8.47 | 3.04 | 19.52 | | | |

TABLE 16
Data from Knight ADRC cohort showing MTBR-tau212 is highly
associated with MTBR-tau243(Rho > 0.9), suggesting
the equivalent performnace to predict tau-PET is obtained.

| | MTBR-tau243 | MTBR-tau243 | MTBR-tau243 |
|---|---|---|---|
| | vs. Amyloid | vs. Amyloid | vs. |
| Spearman r | negative | positive | All |
| r | 0.8653 | 0.9364 | 0.9393 |
| 95% confidence | 0.7958 to | 0.9110 to | 0.9209 to |
| interval | 0.9123 | 0.9547 | 0.9535 |

TABLE 17
Data from BioFINDER 2 showing high correlation
between MTBR-tau243 and MTBR-tau212 (Rho > 0.9).

| | MTBR-tau243 |
|---|---|
| | vs. |
| Spearman r | MTBR-tau212 |
| r | 0.9440 |
| 95% confidence interval | 0.9349 to 0.9519 |

TABLE 18
LOAD60 demographics

| | | |
|---|---|---|
| Amyloid (-) | CDR 0 | n = 13 |
| | CDR 0.5 | n = 3 |
| | CDR 1 | n = 3 |
| Amyloid (+) | CDR 1 | n = 7 |
| | CRD 0.5 | n = 3 |
| | CDR 1 | n = 2 |
| | CDR 2 | n = 1 |
| Amyloid undefined | | n = 27 |

TABLE 19
Summary of endogenously cleaved MTBR-tau243.

| | | | | | Increase in | Increase in |
|---|---|---|---|---|---|---|
| | | | | | low/middle/ | low/medium/ |
| | | | | Increase in | high tau PET | high tau PET |
| Candidate | Sequence | Residue | PTM | AD brain? | CSF? | Plasma? |
| 1 | LQTAPVPMPDLKN | 243-255 | | No increase | No | Not detected |
| 2 | LQTAPVPMPDLK(dN) | 243-255 | Deamidation | Yes | Yes | Not detected |
| | | | at 255 | | | |
| 3 | LQTAPVPMPDLKNVK | 243-257 | | No increase | No | Not detected |
| 4 | LQTAPVPMPDLK(dN)VK | 243-257 | Deamidation | Yes | Yes | Not detected |
| | | | at 255 | | | |
| 5 | LQTAPVPMPDL | 243-253 | | Yes | No | Not detected |
| 6 | LQTAPVPMPDLK | 243-254 | | No increase | No | No |
| 7 | L(dQ)TAPVPMPDLK | 243-254 | Deamidation | No increase | No | Not detected |
| | | | at 244 | | | |
| 8 | L(dQ)TAPVPMPDLK(dN)VK | 243-257 | Deamidation | Not detected | Not detected | Not detected |
| | | | at 244 and 255 | | | |
| 9 | LQTAPVPMPD | 243-252 | | Yes | No | Not detected |
| 10 | LQTAPVPMPDLK(dN)VKS | 243-258 | Deamidation | Yes | Yes | Not detected |
| | | | at 255 | | | |
| 11 | LQTAPVPMPDKLNV | 243-256 | — | Yes | Yes | Yes |
| 12 | LQTAPVPMPDLK(dN)V | 243-256 | Deamidation | Yes | Yes | Yes |
| | | | at 255 | | | |

### Association Between CSF Marker and Amyloid or Tau Measure

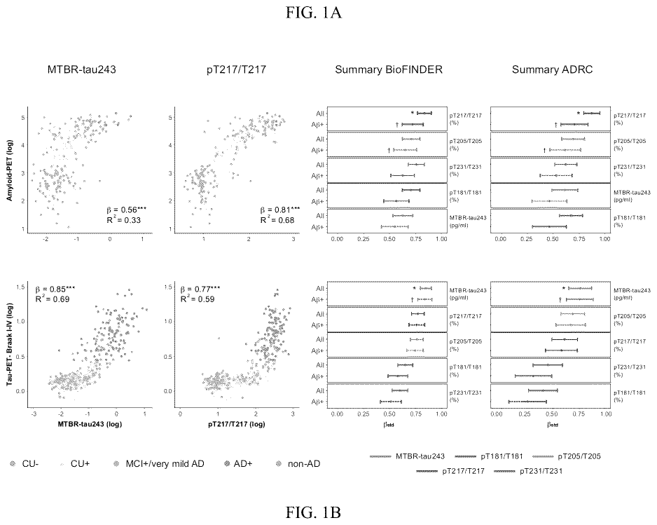

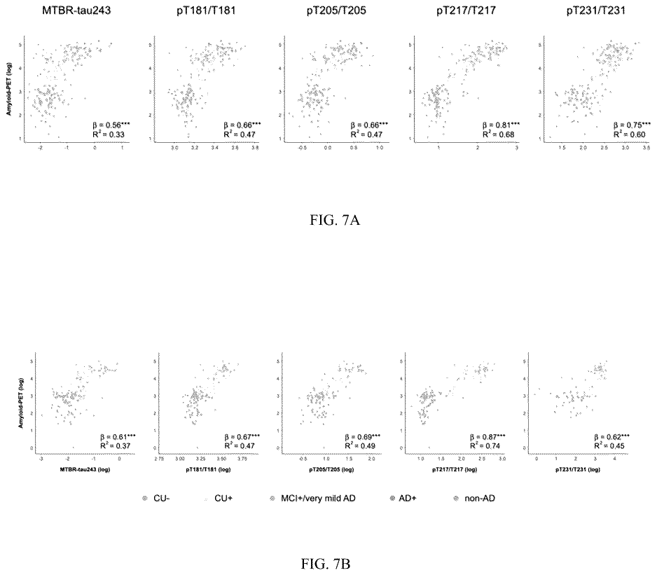

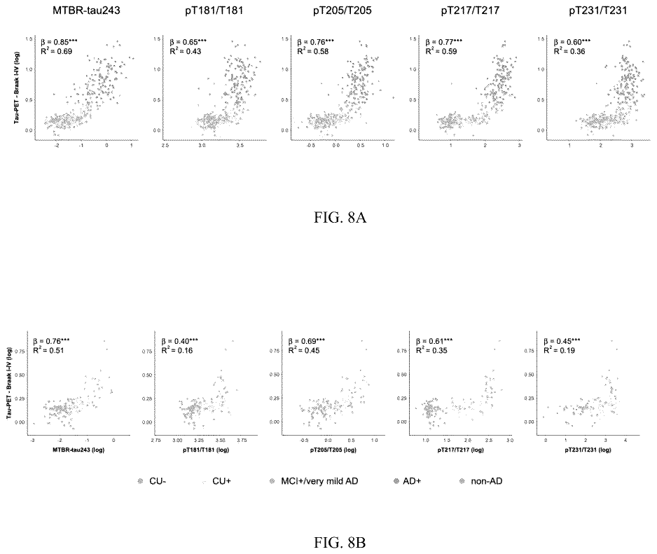

CSF MTBR-tau243, pT181/T181, pT205/T205, pT217/T217, and pT231/T231 were assessed for association with amyloid- and tau-PET measures of pathology using linear regression models adjusting for age and sex. All participants were compared, in addition to the amyloid positive only subgroup, in order to separate out amyloid from tau pathology effects (FIGS. 1A-B). The phosphorylation occupancy at T217 (pT217/T217) was the CSF measure most strongly correlated with amyloid-PET (BioFINDER-2: β [95% CI]=0.81 [0.74, 0.88]; Knight ADRC: β=0.87 [0.79, 0.95]; all p<0.001, FIG. 1A and Table 4). MTBR-tau243 concentration was the CSF measure most strongly associated with tau-PET in all participants (BioFINDER-2: β=0.85 [0.80, 0.90]; Knight ADRC: β-0.76 [0.65, 0.87]; all p<0.001) and amyloid positive participants (BioFINDER-2: β=0.84 [0.77, 0.91]; Knight ADRC: β=0.76 [0.63, 0.89]; all p<0.001, FIG. 1B and Table 4). Notably, the CSF MTBR-tau243 concentration was significantly more strongly associated with tau-PET when compared to pT217/T217 (BioFINDER-2: β-0.77 [0.71, 0.83]; Knight ADRC: β=0.61 [0.49, 0.73]) in all participants and amyloid positive participants (BioFINDER-2: β=0.76 [0.69, 0.84]; Knight ADRC: β=0.58 [0.43, 0.73]; Table 4). Scatter plots for the associations of all CSF tau biomarkers and amyloid- and tau-PET in both cohorts are shown in FIGS. 7A-B and 8A-B, respectively.

We also investigated correlations of CSF tau measures with CSF Aβ42/40. Of the CSF tau measures, pT217/T217 was most strongly correlated with CSF Aβ42/40 (BioFINDER-2: β[95% CI]=−0.80 [−0.86, −0.74]; Knight ADRC: β=−0.88 [−0.95, −0.81]; all p<0.001, FIGS. 12A-B and Table 8). In contrast, MTBR-tau243 concentration showed significantly lower association with CSF Aβ42/40 compared to pT217/T217 (BioFINDER-2: β=−0.63 [−0.70, −0.55]; Knight ADRC: β=−0.59 [−0.71, −0.47]; all p<0.001, FIGS. 12A-B and Table 8).

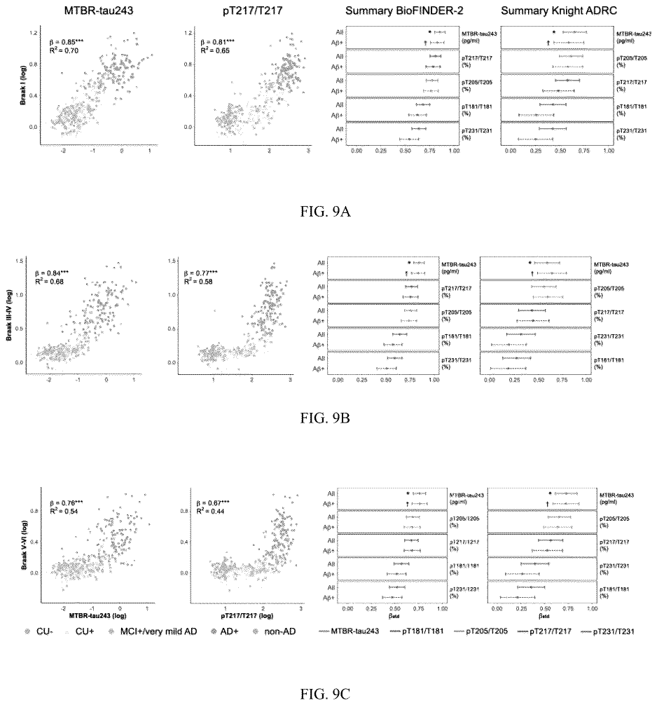

Correlations of CSF tau measures and tau-PET signal in different Braak regions (entorhinal [Braak I], temporal [Braak III-IV], and neocortical [Braak V-VI]) were also investigated as an additional analysis. Comparisons in the amyloid positive only group demonstrated that CSF MTBR-tau243 had the highest correlations with all Braak regions (BioFINDER-2: β=0.85, 0.84, and 0.76; Knight ADRC: β=0.83, 0.84, and 0.76 for each Braak regions respectively; all p<0.001, FIGS. 9A-C and Table 9).

### Biomarker Variation Explained by Amyloid and Tau Pathologies

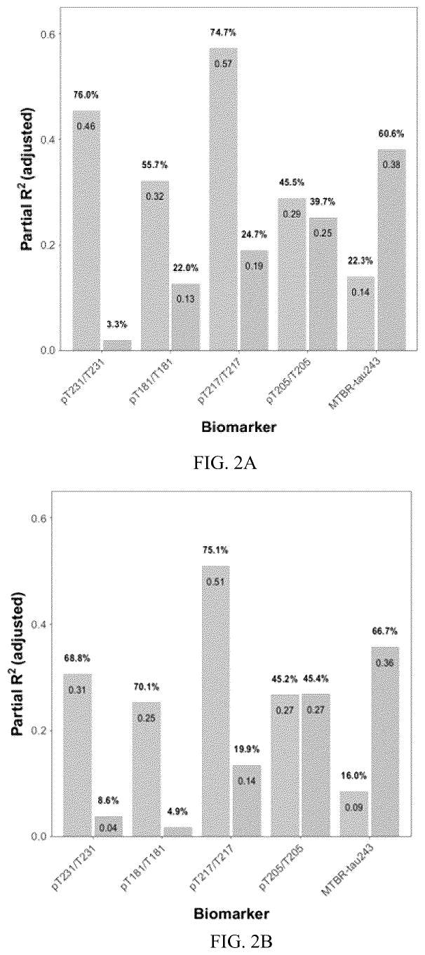

Next, we evaluated the proportion of variation in CSF biomarkers explained by amyloid and tau pathologies. CSF biomarker levels were included as the outcome and amyloid-PET and tau-PET were both included as predictors controlling for age and sex, in our models. In the BioFINDER-2 cohort, variance in CSF pT217/T217 levels was significantly better explained by Aβ pathology as assessed with amyloid-PET, than tau (Aβ: partial R² [pR²]=0.57, 74.7% R2 vs tau: pR²=0.19, 24.7% R², p<0.001, FIG. 2A and Table 5). CSF pT231/T231 (Aβ: pR²=0.46, 76.0% R²; tau: pR²=0.02, 3.3% R², p<0.001) and pT181/T181 (Aβ: pR²=0.32, 55.7% R2; tau: pR2=0.13, 22.0% R2, p=0.006) were also significantly better explained by Aβ pathology. In contrast, variance in CSF MTBR-tau243 concentrations were significantly better explained by tau pathology (Aβ: pR2=0.14, 22.3% R2; tau: pR2-0.38, 60.6% R2, p<0.001). The contribution of tau and amyloid to CSF pT205/T205 was similar, with the difference between both being non-significant (Aβ: pR2=0.29, 45.4% R2; tau: pR2=0.25, 39.7% R2, p=0.657).

Similar trends were observed in the Knight ADRC cohort, although with a greater proportion of variance was explained by Aβ pathology for all CSF biomarkers, likely because this cohort included relatively few individuals with significant tau pathology (only n=36 [16.4%] were tau-PET positive). CSF pT217/T217 (Aβ: pR2-0.51, 75.1% R2; tau: pR2=0.14, 19.9% R2, p<0.001), pT181/T181 (Aβ: pR2-0.25, 70.1% R2; tau: pR2=0.02, 4.9% R2, p<0.001) and pT231/T231 (Aβ: pR2-0.31, 68.8% R2; tau: pR2=0.04, 8.6% R2, p<0.001) were better explained by Aβ pathology. In contrast, tau pathology was the major contributor on explaining variance in CSF MTBR-tau243 levels (Aβ: pR2=0.09, 16.0% R2; tau: pR2-0.36, 66.7% R2, p<0.001). Of note, pT205/T205 levels were explained similarly by both tau and amyloid (Aβ: pR2-0.27, 45.2% R2; tau: pR2=0.27, 45.4% R2, p=0.990, FIG. 2B and Table 5).

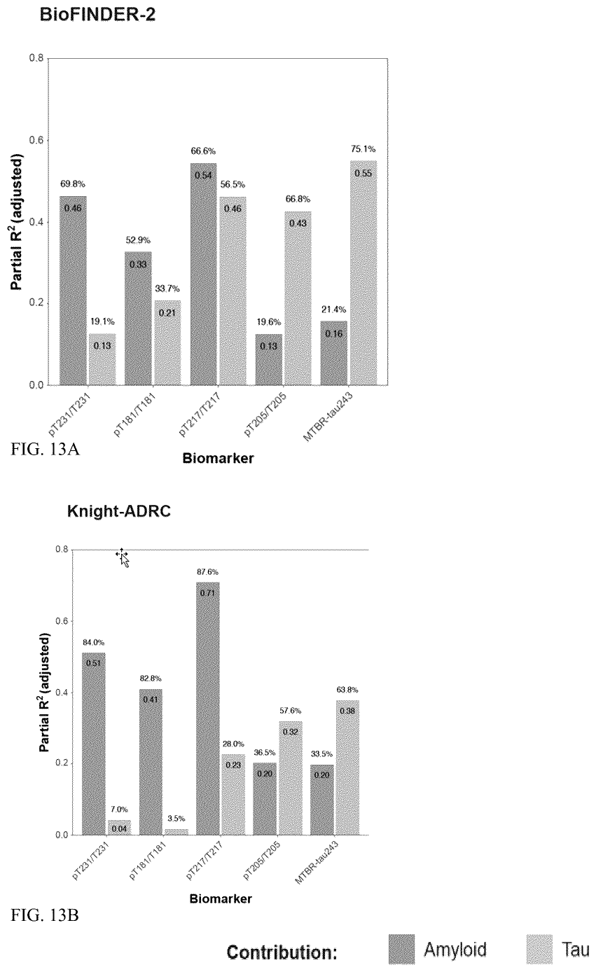

Because dementia patients of BioFINDER-2 did not undergo amyloid-PET, analyses were repeated in both cohorts with all participants using CSF Aβ42/40 rather than amyloid-PET as the measure of Aβ pathology (FIG. 13A-B and Table 5). Levels of CSF pT217/T217 were slightly, but significantly better explained by CSF Aβ42/40 levels than tau-PET (BioFINDER-2:66.6% R2 vs. 56.5%, p=0.044; Knight ADRC: 87.6% R2 vs. 28.9%, p<0.001). CSF Aβ42/40 continued to be the major factor associated with CSF pT231/T231 (BioFINDER-2:69.8% vs. 19.1%, p<0.001; Knight ADRC: 84.0% vs. 7.0%, p<0.001) and pT181/T181 (BioFINDER-2: 52.9% vs. 33.7, p=0.014; Knight ADRC: 82.8% vs. 3.5%, p<0.001). In these models, tau pathology remained the major factor explaining variance in MTBR-tau243 levels (BioFINDER-2:21.4% vs. 75.1%, p<0.001; Knight ADRC: 33.5% vs. 63.8%, p=0.014). For pT205/T205, the major contributor was tau pathology (BioFINDER-2:19.6% vs. 66.8%, p<0.001) although the difference was not significant in Knight ADRC (36.5% vs. 57.6%, p=0.125).

### Longitudinal Change in CSF Biomarkers

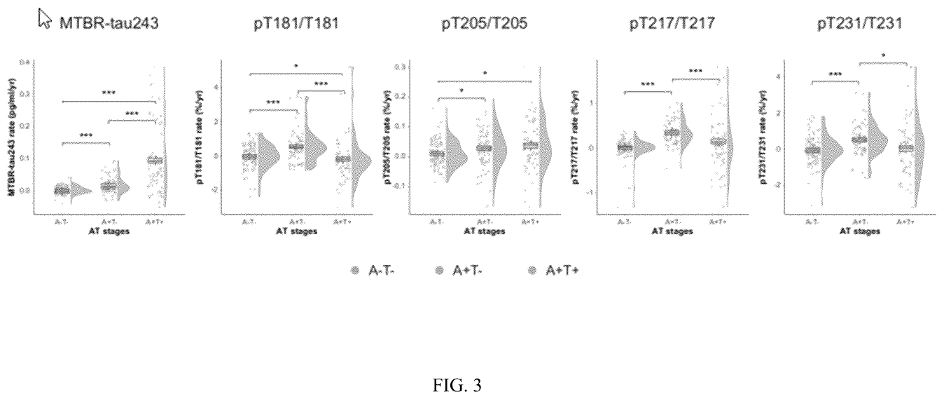

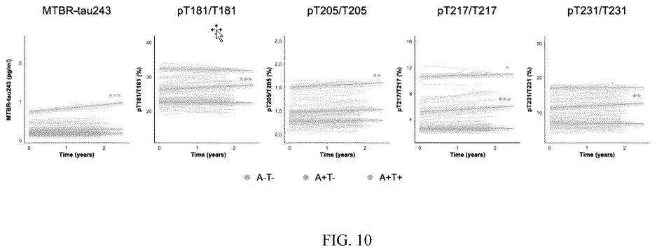

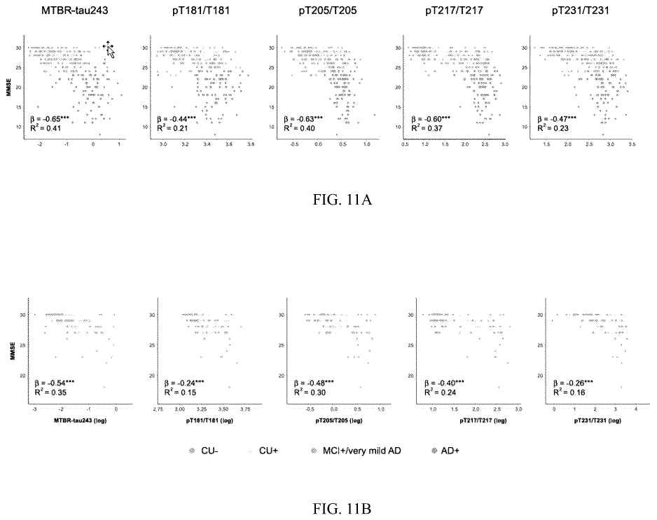

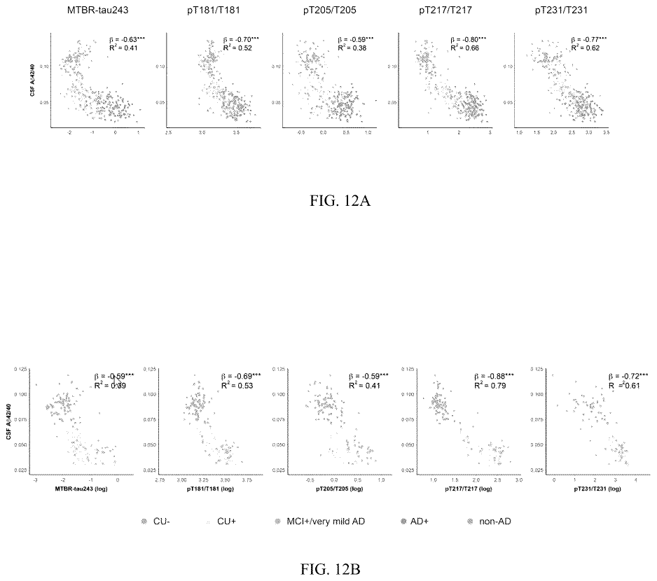

Longitudinal data from the BioFINDER-2 cohort was used to examine changes in CSF biomarkers stratified by amyloid (A) and tau (T) pathology status (positive: +, negative: −). Characteristics of the 220 participants with longitudinal CSF measurements are described in Table 10. Linear mixed models were used to compare CSF longitudinal trajectories among groups (i.e., A−/T−, A+/T− and A+/T+) using post-hoc pairwise Wilcoxon test when the interaction with time was significant. Amyloid positive vs. negative was derived from CSF Aβ42/40 levels and tau was dichotomized from tau-PET measures. Individual and group trajectories over time are shown in FIG. 10. CSF pT217/T217, pT181/T181, and pT231/T231 had their greatest rate of increase in the A+T− group, and a lower rate of increase in the A+T+ group, indicating that the rate of increase of these biomarkers was plateauing at later stages of disease when tau pathology was increasing most (FIG. 3). In contrast, CSF pT205/T205 and MTBR-tau243 had their greatest rate of increase in the A+T+ group, corresponding to matching increases in tau pathology. Importantly, CSF MTBR-tau243 was increasing faster in the A+T+ group than the A+T− group (vs A−T−: Cohen's d=1.48, p<0.001; vs A+T−: Cohen's d=1.13, p<0.001), while the rate of increase in pT205/T205 was not significantly different in the A+T+ and A+T− groups (Cohen's d=0.08, p=0.788, FIG. 3 and Table 11). This suggests that CSF MTBR-tau243 would best reflect AD progression in tau-PET positive individuals.

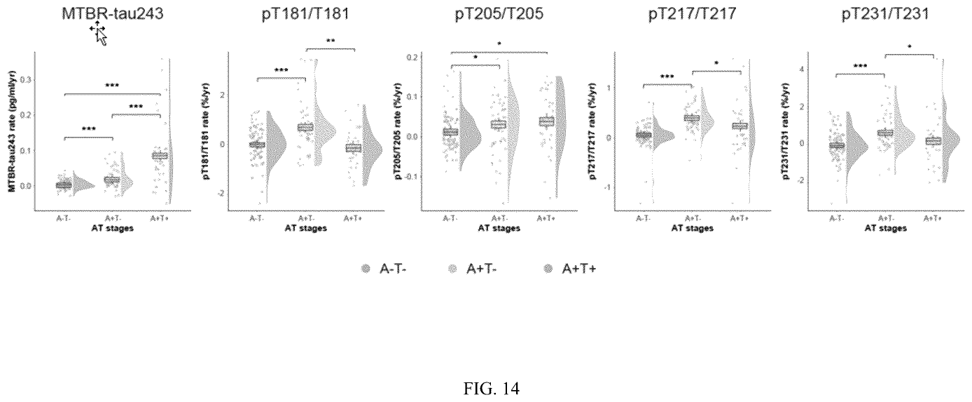

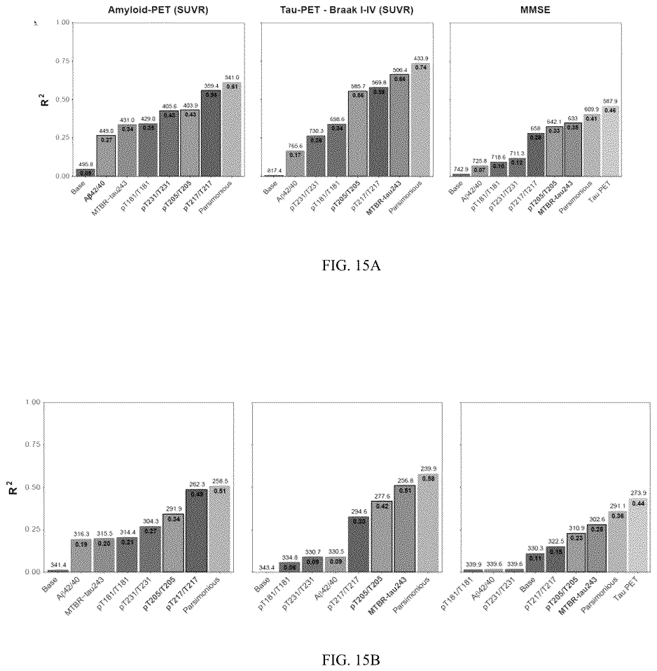

As a sensitivity analysis, we repeated this analysis using amyloid-PET rather than CSF Aβ42/40 for classifying participants, using a previously validated threshold³⁹. We found that the longitudinal trajectories for all CSF biomarkers were replicated, with pT205/T205, but especially MTBR-tau243, rates of change increasing with progressing A/T status, and the rest of biomarkers having the highest rate of change at A+T− status (FIG. 14 and Table 11).

Association of CSF and PET Biomarkers with MMSE Scores

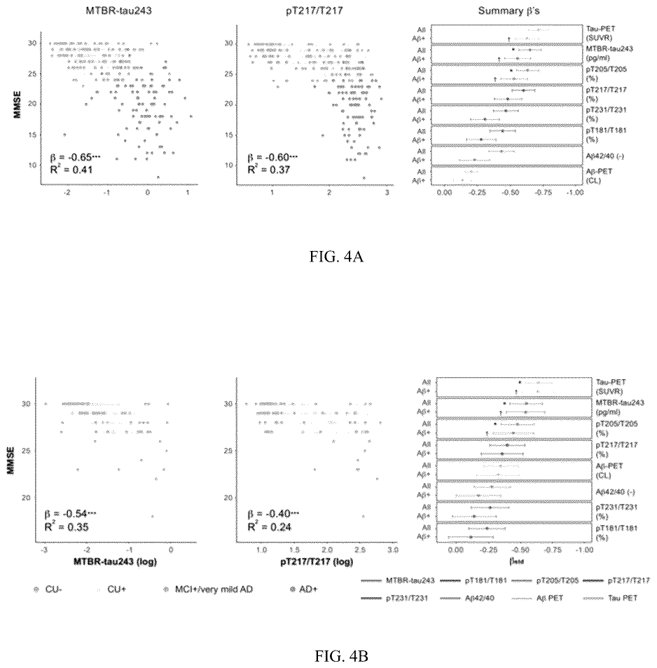

We assessed associations of CSF and PET biomarkers with a common clinical assessment of dementia, the Mini Mental State Examination (MMSE) 40, which were assessed in both cohorts, using linear regression models that adjusted for age, sex, and years of education. MTBR-tau243 was the CSF biomarker most strongly associated with MMSE scores in all participants (BioFINDER-2: β=−0.65 [−0.74, −0.57]; Knight ADRC: β=−0.54 [−0.67, −0.42], all p<0.001) and amyloid positive participants (BioFINDER-2: β=−0.56 [−0.66, −0.46]; Knight ADRC: β=−0.54 [−0.69, −0.39], all p<0.001, FIG. 4 and Table 12). These associations were significantly stronger than those of pT217/T217, as assessed by bootstrapping, for all participants (BioFINDER-2: β=−0.60 [−0.69, −0.52], p comparison of β=0.001; Knight ADRC: β=−0.40 [−0.53, −0.26], p comparison of β=0.003) and amyloid positive participants (BioFINDER-2: β=−0.48 [−0.59, −0.38], p comparison=0.002; Knight ADRC: β=−0.36 [−0.52, −0.19], p comparison=0.001). However, tau-PET was more strongly associated with MMSE than any CSF biomarker (BioFINDER-2: β=−0.73 [−0.79, −0.64], p comparison=0.036; Knight ADRC: β=−0.64 [−0.74, −0.53], p comparison<0.001). Scatter plots for each CSF biomarker are shown in FIGS. 11A-B.

### Best Predictors of AD-Related Measures and Cognitive Function

Finally, we aimed to determine whether combinations of CSF biomarkers could be used as accurate quantitative surrogates for amyloid-PET, tau-PET, or cognitive measures. We first evaluated at the variance explained by each individual biomarker for each outcome. Next, we used the least absolute shrinkage and selection operator (LASSO) procedure to select which combination of CSF biomarkers were optimal for each outcome and then, we compared this new model to the ones from the individual biomarkers.

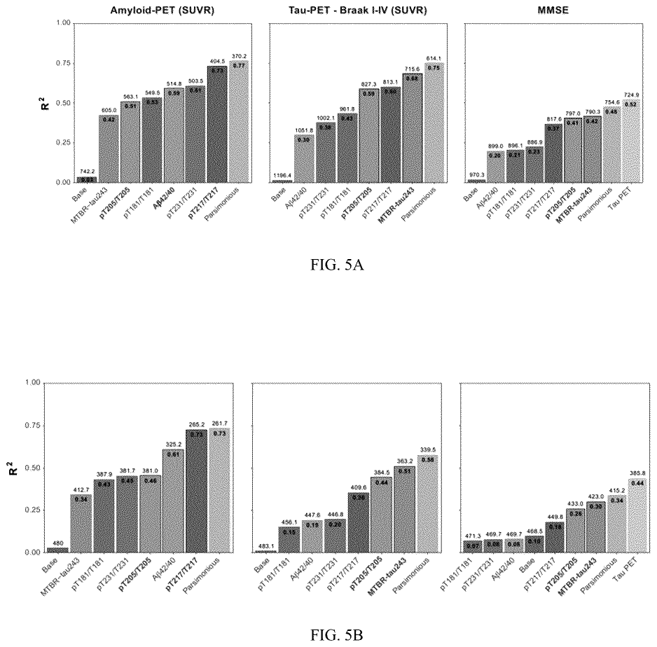

CSF pT217/T217 was the individual biomarker that best predicted amyloid-PET (BioFINDER-2: R²=0.73, AICc=404.5; Knight ADRC: R²=0.73, AICc=265.2, FIG. 5 and Table 13). Based on the LASSO regressions, we found that combining CSF pT217/T217 with pT205/T205 and Aβ42/40 significantly improved prediction of amyloid-PET in both cohorts (BioFINDER-2: R²=0.77, AICc=370.2, F=20.974, p<0.001; Knight ADRC: R²=0.73, AICc=261.7, F=5.266, p=0.006, Table 6).

MTBR-tau243 was the individual biomarker that best predicted tau-PET (BioFINDER-2: R²=0.68, AICc=715.6; Knight ADRC: R²=0.51, AICc=363.2, FIG. 5 and Table 13). The optimal model, which combined MTBR-tau243 and pT205/T205, significantly improved prediction of tau-PET amounts in both cohorts (BioFINDER-2: R²=0.75, AICc=614.1, F=116.49, p<0.001; Knight ADRC: R2-0.58, AICc=339.5, F=30.268, p<0.001, FIG. 5 and Table 6).

MTBR-tau243 was the individual biomarker that best predicted MMSE scores (BioFINDER-2: R²=0.42, AICc=790.3; Knight ADRC: R²=0.30, AICc=423.0 FIG. 5 and Table 13), and prediction improved when pT205/T205 was added (BioFINDER-2: R²=0.48, AICc=754.6, F=27.693, p<0.001; Knight ADRC: R²=0.34, AICc=415.2, FIG. 5 and Table 6). Tau-PET predicted MMSE scores better than a combination of CSF biomarkers, but the difference was not statistically significant (BioFINDER-2: R²=0.52, AICc=724.9, z=0.900, p=0.184; Knight ADRC: R²=0.44, AICc=385.8, z=−1.405, p=0.080, FIG. 5). Results for amyloid positive only participants demonstrated similar results (FIGS. 5A-B and Table 12 and Table 6).

### DISCUSSION

In this study, we found that a novel CSF biomarker, MTBR-tau243, was strongly associated with tau pathology, while minimally associated with Aβ-pathology, in two large independent sporadic AD cohorts. We also found that CSF MTBR-tau243 has a significantly higher correlation with cognitive measures than phosphorylated tau measures (e.g., pT217/T217 and pT181/T181), which indicates its potential utility in the clinical setting. Further, we found that CSF MTBR-tau243 is the biomarker with the largest rate of increase in participants that are already positive for both amyloid and tau pathologies, suggesting that CSF MTBR-tau243 best reflects disease progression in late stages. We further extended these findings by combining CSF MTBR-tau243 with phosphorylated tau measures to predict Aβ pathology, tau pathology, and cognitive measures in the AD continuum. We found that CSF MTBR-tau243 in combination with pT205/T205 can accurately predict continuous tau-PET measures and has similar predictive accuracy for cognitive measures as tau-PET. Based on these results, our study suggests that CSF MTBR-tau243 may be a viable alternative to tau-PET for use as a pre-screening tool or a tau pathology endpoint surrogate for clinical trials, and also as an accurate diagnostic measure of tau pathology.

Our first objective was to characterize MTBR-tau243 concentration and compare it to four phosphorylated tau measures by looking at their associations with Aβ and tau pathologies measured by PET. Notably, MTBR-tau243 was the tau biomarker that demonstrated the highest correlation with tau-PET and the lowest correlation with amyloid-PET, not only in the total group, but also in Aβ-positive group. This suggests that MTBR-tau243 is a biomarker that specifically reflects aggregated tau pathology independent of amyloid pathology. Although pT217/T217 was also well correlated with tau-PET, there was a nonlinear relationship and a substantial increase in pT217/T217 before tau-PET pathology was elevated, which plateaued once the tau-PET threshold was exceeded. This may indicate that pT217/T217 is primarily associated with tau pathology through its quantitative relationship with the amount of Aβ pathology. This is further supported by the observation that Aβ pathology explained a significantly larger proportion of variation of pT217/T217 levels than tau, when including both amyloid-PET and tau-PET measures in the model. Interestingly, while pT205/T205 levels demonstrated a high correlation with tau-PET, it also showed the second highest correlation with amyloid-PET, after pT217/T217. In combined models, both amyloid- and tau-PET explained similar proportion of variation of pT205/T205 levels, suggesting that it is an intermediate biomarker affected by both Aβ and tau pathologies. Regarding the other p-tau measures, pT181/T181 and pT231/T231 were highly correlated with amyloid-PET, while the correlations with tau-PET were significantly lower than the other three CSF tau biomarkers, suggesting that they mainly reflect Aβ-pathology. These results are in line with several recent studies suggesting that p-tau181, p-tau217 and p-tau231 may be more related to amyloid pathology than tau. This is supported by their increased levels, both measured in CSF or in plasma, in early stages^{13,14,17,21,22,41-43}, to be more tightly associated with amyloid-PET than tau-PET^{18,23,44} or to actual amyloid pathology in post-mortem studies^{45,46} Finally, we found that MTBR-tau243 was particularity increased in two cases of MAPT R406W mutation carriers that were amyloid negative but had high tau-PET binding. Tau pathology on MAPT R406W mutation carriers is known to be similar to AD tau pathology^{47,48} and reactive to AD tau-PET tracers^{4,49-51}, further supporting our finding that MTBR-tau243 is a specific biomarker to AD-like tau pathology.

Longitudinal CSF biomarkers changes were also investigated to understand how these biomarkers change at different stages of the disease. Most interestingly, among the five CSF tau biomarkers, only MTBR-tau243 exhibited a significant increase in the rate of change between A+T− and A+T+ groups, suggesting that it enables longitudinal disease tracking during the phase of the disease characterized by neocortical tau aggregates, which mainly occurs in the symptomatic phase of AD. On the other hand, there was no major difference in the rate of change between A+T− and A+T+ for pT205/T205, although it still demonstrated a positive rate of changes at this late stage, suggesting a lower but still significant increase after tau deposition. Interestingly, for the other phosphorylated tau measures (pT181/T181, pT217/T217, and pT231/T231), there was a pronounced increase in the rate of change during the transition from A−T− to A+T−, consistent with a prior report showing that phosphorylated tau (especially p-tau217) is an optimal marker for disease monitoring during the very early (preclinical) stages of the disease⁵². Interestingly, here we found either no significant increase in the rate of change of phosphorylated tau occupancy during the transition from A+T− to A+T+ or a significant decrease in the rate of change, consistent with prior reports²¹. These results suggest that rate of change in these phosphorylated tau measures may plateau or decline at advanced disease stages, when insoluble tau aggregates are depositing in the neocortex, indicating they are discordant longitudinally and the classic p-tau measures are not direct measures of AD tau pathology⁵³. Altogether, the findings of CSF p-tau measures are consistent with previous clinical observational studies and preclinical mouse models^{22,32}, where these biomarkers seem to be driven by Aβ pathology. These results further support recent proposals to revise the A/T/(N) criteria system, in which any p-tau biomarker can be used as a tau (T) marker⁵⁴.

As a relevant question for clinical practice, we also investigated the relationship between these CSF biomarkers and a cognitive measure. As expected by the observed associations with tau pathology, MTBR-tau243 was the measure most strongly associated with MMSE, a cognitive test frequently used in the clinical setting. Notably, this association was not significantly different from tau-PET, thus supporting the idea that CSF MTBR-tau243 could be a viable alternative to tau-PET for clinical purposes. Although pT205/T205 had a lower correlation with MMSE than MTBR-tau243, pT205/T205 was also well correlated with MMSE and not significantly different from tau-PET. In contrast, other CSF biomarkers such as pT217/T217 or pT181/T181 showed significantly lower associations.

An unmet need is to determine not just who has amyloid or tau pathology, but if the symptoms are due to those pathologies. Because tau pathology is most highly correlated with cognitive and clinical impairment, an important question is how well CSF biomarkers can predict tau pathology or cognitive impairment. We next examined whether combining CSF biomarkers would improve prediction of Aβ or tau pathologies or cognitive measures. We evaluated the predictive performance of the combination of these CSF tau biomarkers for amyloid-PET burden, tau-PET burden, and MMSE. Based on a data-driven approach, we observed that the combination of pT205/T205, Aβ42/40 and pT217/T217 was optimal for predicting amyloid-PET continuous measures and significantly improved the performance of any individual measure. We also found that the combination of MTBR-tau243 and pT205/T205 in a single model improved prediction of tau-PET burden compared to any other single fluid biomarker. The fact that such high predictive accuracy for both amyloid- and tau-PET imaging can be achieved by CSF biomarkers indicates that CSF assays can potentially be an alternative to PET measures, which are costly and have limited accessibility. Notably, MTBR-tau243 and pT205/T205 were also the optimal combination for predicting a cognitive measure (i.e., MMSE), suggesting potential clinical applications of this biomarker combination in predicting not only tau pathology but also cognitive impairment. For broader use, the translation of these biomarkers into blood-based biomarkers will be of utmost importance.

The main strength of this study is that we replicated our key findings in two large independent cohorts that represented different types of populations and used different PET tracers, and also that we measured collected samples prospectively together with pre-defined outcome measures. Although further research is needed in a more diverse and generalizable population to implement our findings in the clinic, it is important to highlight that BioFINDER-2 participants were consecutively recruited from a secondary Clinical Memory in Sweden. As such, this cohort is a representative of memory clinical patients in Sweden and include both AD and also non-AD dementia patients. Limitations include that the magnitude of the trend differed between the two cohorts in some analyses although the similar trends were shown. Potential reasons include that the BioFINDER-2 cohort includes more tau-PET positive participants with AD dementia than the Knight ADRC cohort, as well as more participants in advanced stages of the disease, which may have affected the results with tau-PET and MMSE. Another limitation is that relatively few participants with AD dementia in the BioFINDER-2 cohort had an amyloid-PET scan per study design, although these participants all had CSF Aβ42/40. Thus, we used CSF Aβ42/40 instead of amyloid-PET as a marker of Aβ pathology in a sensitivity analysis and confirmed that this limitation did not affect the overall results and interpretations. Further, we acknowledge that our measures of Aβ and tau pathologies are only surrogate biomarkers and not actual measures of pathology, but both amyloid- and tau-PET markers have been validated against neuropathological measures of insoluble Aβ and tau aggregates, respectively^{2,3,6,55-58}. Future studies using animal models and neuropathological measures will be important to further validate the results here presented.

In conclusion, these findings confirm that CSF MTBR-tau243 specifically reflects changes in aggregated tau pathology that occur at a later stage of AD progression and are associated with clinical and cognitive symptoms. Thus, we suggest that MTBR-tau243 should replace the commonly used p-tau measures as the fluid biomarker representing insoluble tau aggregate pathology (T) in defining AD pathology and in future versions of the commonly used A/T/(N) criteria for AD⁵³. As such, MTBR-tau243 could be used to assess AD tauopathy and track the effects of drug treatment independent of amyloid effects. The combination of CSF MTBR-tau243 and pT205/T205 is nearly equivalent to tau-PET measures and predicts MMSE almost as accurately as tau-PET, which indicates clinical utility of a biomarker panel containing MTBR-tau243. Compared to biomarkers altered by amyloidosis that are often abnormal in older cognitively normal individuals, CSF MTBR-tau243 could enable confirmation of tau pathology and provide greater certainty that cognitive symptoms are due to AD, as proposed in the latest clinical AD criteria requiring biomarker evidence of both amyloid and tau pathology to diagnose AD with high likelihood⁵⁹. These findings add to the improving biomarker diagnostic accuracy for AD and to strategies to develop novel AD therapies.

### Methods:

### Participants

Participants were included from two cohorts: the Swedish BioFINDER-2 (NCT03174938)¹³ at Lund University (Lund, Sweden), and the Knight ADRC from Washington University (St Louis, MO, USA). The BioFINDER-2 cohort included cognitively unimpaired participants (recruited as cognitively normal controls or as subjective cognitive decline [SCD] patients), patients with MCI, AD dementia patients and patients with a non-AD neurodegenerative disease. Participants were recruited at Skåne University Hospital and the Hospital of Ängelholm in Sweden. Details on recruitment, exclusion and inclusion criteria have been presented before¹³. All participants underwent lumbar puncture at baseline and at the follow-up after two years for CSF sampling. Participants underwent cognitive testing, including MMSE. The Knight ADRC cohort consisted of community-dwelling volunteers enrolled in studies of memory and aging at Washington University in St. Louis. All Knight ADRC participants underwent a comprehensive clinical assessment that included a detailed interview of a collateral source, a neurological examination of the participant, the Clinical Dementia Rating® (CDR)⁶⁰ and the Mini-Mental State Examination (MMSE)⁴⁰. Individuals with a CDR of 0.5 or greater were considered to have a dementia syndrome and the probable aetiology of the dementia syndrome was formulated by clinicians based on clinical features in accordance with standard criteria and methods⁶¹.

In the BioFINDER-2 cohort, participants were divided in CU either Aβ negative or positive (CU− and CU+, respectively), MCI patients Aβ positive (MCI+), AD dementia patients Aβ positive (AD+), or non-AD neurodegenerative patients, regardless of their Aβ status. Two participants (one in CU− and the other in non-AD) were MAPT R406W mutation carriers with Aβ negative and tau positive. In the Knight ADRC cohort, participants were divided in CU with CDR=0 either Aβ negative or positive (CU− and CU+, respectively), very mild AD patients with CDR=0.5 Aβ positive, AD dementia patients with CDR≥1 Aβ positive (AD+). In accordance with the research framework by the National Institute on Aging-Alzheimer's Association study patients with SCD and cognitively healthy controls were considered the CU group⁵. All participants gave written informed consent and ethical approval was granted by the Regional Ethical Committee in Lund, Sweden and the Washington University Human Research Protection Office, respectively.

### Anti-Tau Antibody Generation

Antibodies HJ32.11 and HJ34.8 were generated by immunizing tau knockout mice (Jackson labs) with either keyhole limpet haemocyanin (KLH) fused to amino acids 225-242 of tau to generate antibody HJ32.11 or to KLH fused to amino acids 226-264 to generate antibody HJ34.8. Spleen cells from immunized mice were fused with P3 hybridoma cells and expanded. Clones were screened by direct enzyme-linked immunosorbent assay.

### CSF Measurements

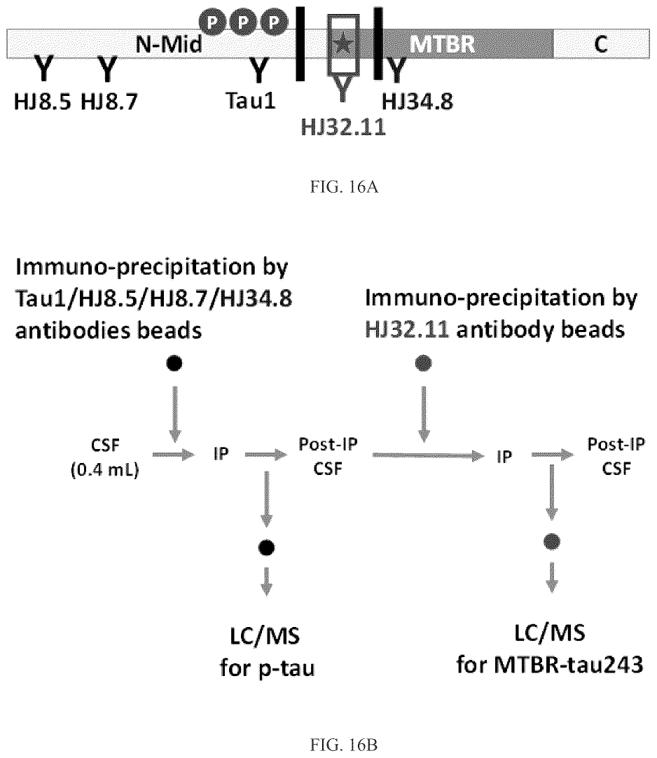

Measurement of CSF tau species including p-tau and MTBR-tau243 was performed at Washington University in both cohorts using the newly developed immunoprecipitation/mass spectrometry (IP/MS) method. We developed two new monoclonal antibodies to immune-purify CSF MTBR-tau243: HJ32.11, which binds near residue 243, and HJ34.8, which binds near residue 260. The procedure of CSF tau analysis is described in FIG. 16. The calculation of percent phosphorylation was performed by measuring the phosphorylated peptide and the non-phosphorylated peptide in the same injection and calculating the percent phosphorylation occupancy as % p-tau/t-tau²¹.

Additionally, CSF Aβ42/40 levels were used in both cohorts to assess Aβ positivity. In the BioFINDER-2 cohort, CSF levels of Aβ42/40 were measured as previously explained¹³. A threshold of 0.080, based on a Gaussian mixture modeling, determined Aβ positivity³⁹. In the Knight ADRC cohort, CSF Aβ42/40 levels were measured as explained previously⁶². The threshold (0.0673) had the maximum combined sensitivity and specificity in distinguishing amyloid-PET status.

### Imaging Acquisition and Quantification

In the BioFINDER-2 cohort, amyloid and tau-PET acquiring methods have been previously reported¹³. Briefly, amyloid-PET was acquired using [¹⁸F]flutemetamol and tau-PET using [¹⁸F]RO948. Of note, most of the AD patients did not undergo amyloid-PET in BioFINDER-2, due to the study design. In the Knight ADRC cohort, participants underwent amyloid-PET using either [¹⁸F]florbetapir ([¹⁸F]AV45) or [¹¹C]PiB and tau-PET with [¹⁸F]flortaucipir ([¹⁸F]AV1451) as previously explained³³. Amyloid-PET was measured in a neocortical meta-ROI using cerebellar grey as a reference region. In the BioFINDER-2 cohort, Centiloids were calculated using the Computational Analysis of PET from AIBL (CapAIBL) pipeline⁶³. For tau-PET, SUVRs were calculated using the inferior cerebellum cortex as reference region and binding from a temporal meta-ROI were used for main analyses (Braak I-IV), in order to capture the regions most affected by tau. In supplementary analyses, we also quantified tau-PET in early (Braak I), intermediate (Braak III-IV) and late (Braak V-VI) regions of tau deposition⁶⁴. Tau positivity was assessed based on tau-PET in all cases. In the Braak I-IV region, cut-off for positivity was set at SUVR>1.32 both in BioFINDER-2 and in the Knight ADRC cohorts^{65,66}.

### Cognitive Tests

MMSE was used as a measure of global cognition in both cohorts.

### Statistical Analyses

Differences in CSF biomarker levels by diagnostic groups were tested using ANCOVA adjusted for age and sex. Post-hoc analyses were performed using the Tuckey test. Linear regression models were used to assess the association between amyloid- and tau-PET (independent variable) and each of the CSF biomarkers (dependent variable), after adjusting for age and sex. For cognition, we additionally used education as covariate in the linear regression models. All standardized betas were compared to the highest for each outcome and cohort, by building a distribution of the betas' difference and using that to infer significance using a bootstrapping approach (n=500) with the boot package. Proportion of variation of CSF levels by amyloid and tau measures were assessed using linear regression models with both amyloid and tau as predictors, CSF levels as outcomes and age and sex as covariates. We calculated the partial R² of amyloid and tau, raw and as a percentage of the total R² of the model using the rsq package. This was used as a measure of proportion of variance explained by amyloid and tau. Next, prediction of amyloid and tau continuous measures was assessed with linear regression models, where amyloid- and tau-PET measures were used as outcomes in independent models and individual CSF biomarkers as predictors. A basic model was also created with only covariates (age and sex) as predictors. Additionally, a parsimonious model was constructed to optimally predict (highest accuracy with lower number of predictors) each of these measures, independently for each cohort. To this aim, LASSO regression models were used (glmnet package), initially including all CSF biomarkers and covariates. Only those predictors selected by the LASSO regression and with a significant contribution (p<0.1) in the model were finally included in the parsimonious model. Similar methods were used for predicting cognition (MMSE in the two cohorts and CDR in Knight ADRC) additionally including education as covariate. In these cases, we compared the parsimonious model to one including only tau-PET as predictor. F-tests were used to compare nested models (including the same subset of predictors). When comparing models with different predictors we used the Vuong's test using the nonnest2 package. Finally, CSF longitudinal changes by baseline amyloid and tau status were assessed in the BioFINDER-2 cohort. Individual participant slopes were calculated using linear regression models to calculate rate of change differences (mean percentage change) and compare them between groups using a Kruskal-Wallis test. Further, we created group trajectories with linear mixed models using the Ime4 package for visualization. Here, CSF biomarkers were used as outcome, interaction between time and baseline amyloid and tau status as predictor and age and sex main effects as covariates, using random intercepts and fixed time-slopes due to low number of time points. CSF and amyloid- and tau-PET measures were log-transformed in linear regression analyses. A two-sided P value <0.05 was considered statistically significant. R version 4.1.0 was used for all statistical analyses.

### Example 2

### MTBR-Tau243 is a Fluid Tau Biomarker Specific to Tau Pathology

Two-Step Immunoprecipitation and/or Immunodepletion Method to Detect Cleaved Fragments of MTBR-Tau243

A two-step method with immunodepletion and immunoprecitation was developed to isolate cleaved fragments of MTBR-tau243. The method was tested in CSF and plasma.

### MTBR-Tau 243 Detection in CSF

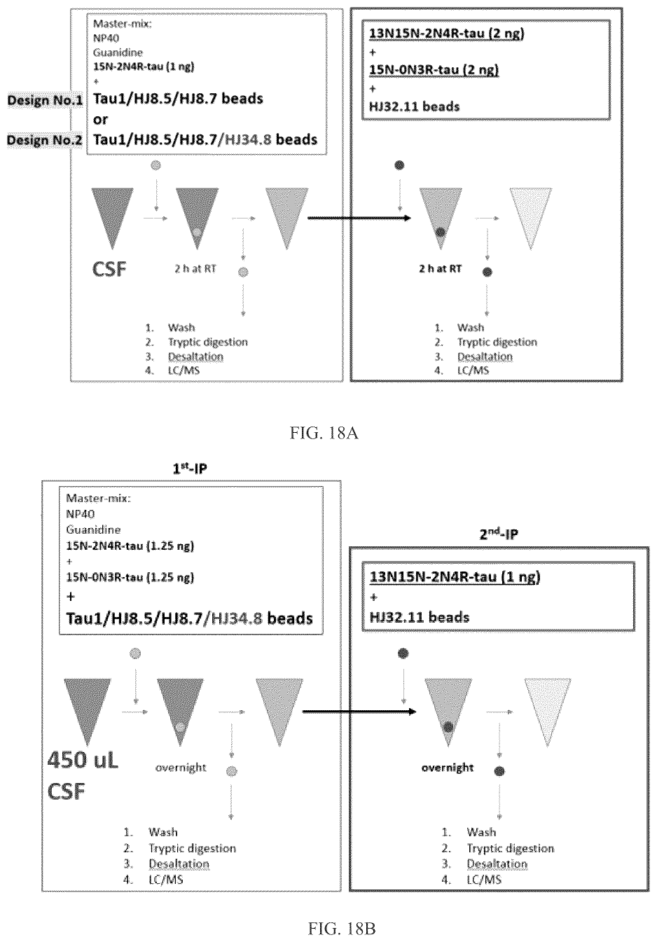

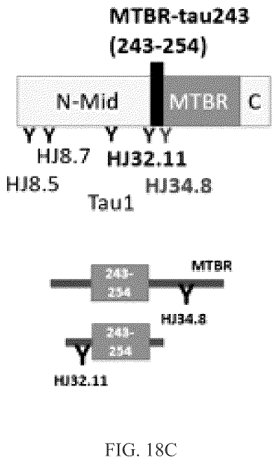

One exemplary method for detecting MTBR-tau243 is CSF is provided in Example 1. FIG. 18A shows two methods of immunodepletion and/or immunoprecipitation. One method (design 1) used Tau1/HJ8.5/HJ8.7 antibodies in a first affinity depletion step. The second method (design 2) used Tau1/HJ8.5/HJ8.7/HJ34.8 antibodies in the first step. For all antibodies, 3 mg/gram sepharose beads were generated. For Tau1 immunoprecipitation (IP), 1.1 ug antibody/sample was used. For HJ8.5 and HJ8.7 IP, 2.3 ug antibody/sample was used. For HJ32.11 and HJ34.8 IP, 11.25 ug antibody/sample was used. As shown in FIG. 18A, a master-mix of NP40, guanidine, 1 ng of ¹⁵N-2N4R-tau (internal standard), and anti-tau antibody beads Tau1/HJ8.5/HJ8.7 (or Tau1/HJ8.5/HJ8.7/HJ34.8) was prepared. The master-mix was added to CSF sample from subjects and incubated for 2 hours at room temperature for affinity depletion (immunodepletion) or immunoprecipitation of MTBR-tau243 resulting in a first immunodepleted precipitate including the antibody bead and CSF solution. The first immunodepleted precipitate was washed and subjected to trypsin for trypsin digestion. The digested sample was subjected to desalted by C18 toptip (Glygen) and analyzed by LC/MS. In a second immunoprecipitateion step, the remaining CSF sample was mixed with a second mixture of 2 ng of ¹³N¹⁵N-2N4R-tau (internal standard), 2 ng of ¹⁵N-0N3R-tau (internal standard), and HJ32.11 antibody beads and incubated for 2 hours at room temperature for affinity depletion (immunodepletion) or immunoprecipitation of MTBR-tau243 resulting in a second immunodepleted precipitate including the HJ32.11 antibody bead. The second immunodepleted precipitate was washed and subjected to trypsin for trypsin digestion. The digested sample was desalted by C18 toptip (Glygen) and analyzed by LC/MS. FIG. 18C shows the MTBR-tau243 peptides affinity to HJ8.5, HJ8.7, HJ34.8, and HJ32.11 antibodies.

As shown in FIG. 18B, a master-mix of NP40, guanidine, 1.25 ng of ¹⁵N-2N4R-tau (internal standard), 1.25 ng of ¹⁵N-0N3R-tau (internal standard) and anti-tau antibody beads Tau1/HJ8.5/HJ8.7/HJ34.8 was prepared. The master-mix was added to 450 μL CSF sample from subjects and incubated for 2 hours at room temperature for affinity depletion (immunodepletion) or immunoprecipitation of MTBR-tau243 resulting in a first immunodepleted precipitate including the antibody bead and CSF solution. The first immunodepleted precipitate was washed and subjected to trypsin for trypsin digestion, which is a form of proteolytic cleavage. The digested sample was desalted by C18 toptip (Glygen) and analyzed by LC/MS. In a second immunoprecipitation step, the remaining CSF sample was mixed with a second mixture of 1 ng of ¹³N¹⁵N-2N4R-tau (internal standard), and HJ32.11 antibody beads and incubated for 2 hours at room temperature for affinity depletion (immunodepletion) or immunoprecipitation of MTBR-tau243 resulting in a second immunodepleted precipitate including the HJ32.11 antibody bead. The second immunodepleted precipitate was washed and subjected to trypsin for trypsin digestion. The digested sample was desalted by C18 toptip (Glygen) and analyzed by LC/MS.

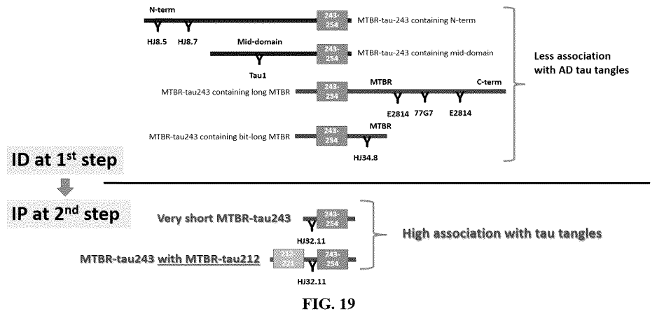

FIG. 19 shows the different MTBR-tau fragments isolated by HJ34.8 antibody bead in the first immunodepletion step and by anti-tau antibody HJ32.11 bead in the second immunoprecipitation step. Long MTBR-tau243 fragments showed affinity for HJ38.8 antibodies and exhibited a reduced association with AD tau tangles. Notably, very short MTBR-tau243 fragments showed affinity for HJ32.11 antibodies and exhibited a high association with tau tangles.

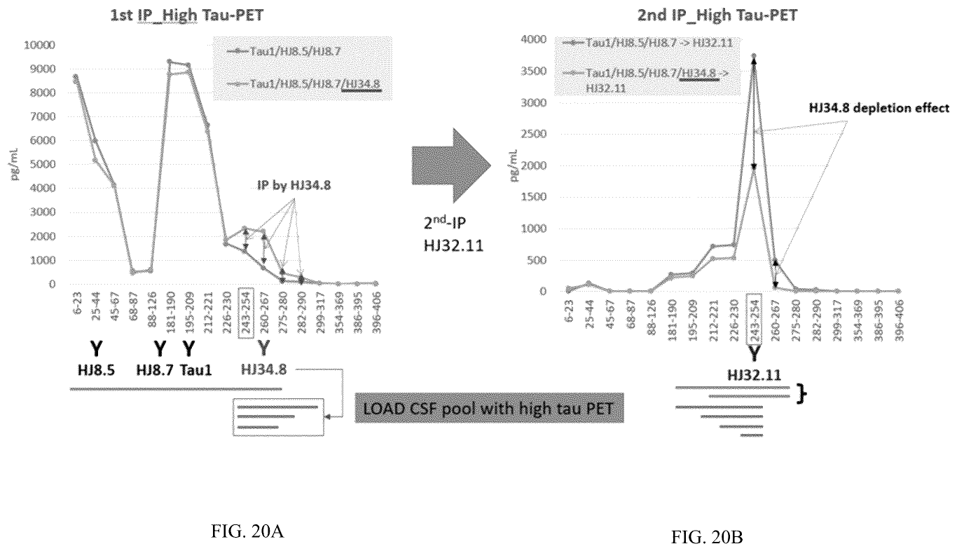

The amount of cleaved MTBR fragments precipitated out by each antibody during the immunoprecipitation and/or immunodepletion is shown in FIGS. 20A-B. FIG. 20A shows HJ34.8 antibody precipitated additional cleaved MTBR-tau243 fragments in the first immunodepletion step. FIG. 20B confirms the immunodepletion of cleaved MTBR-tau243 fragments by HJ34.8.

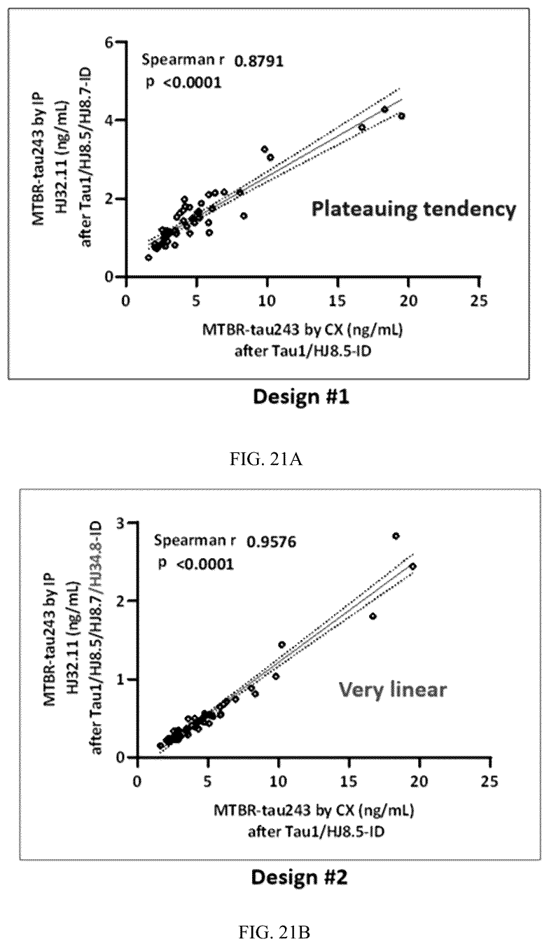

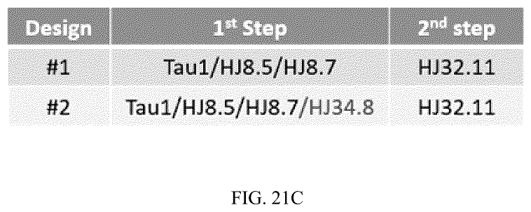

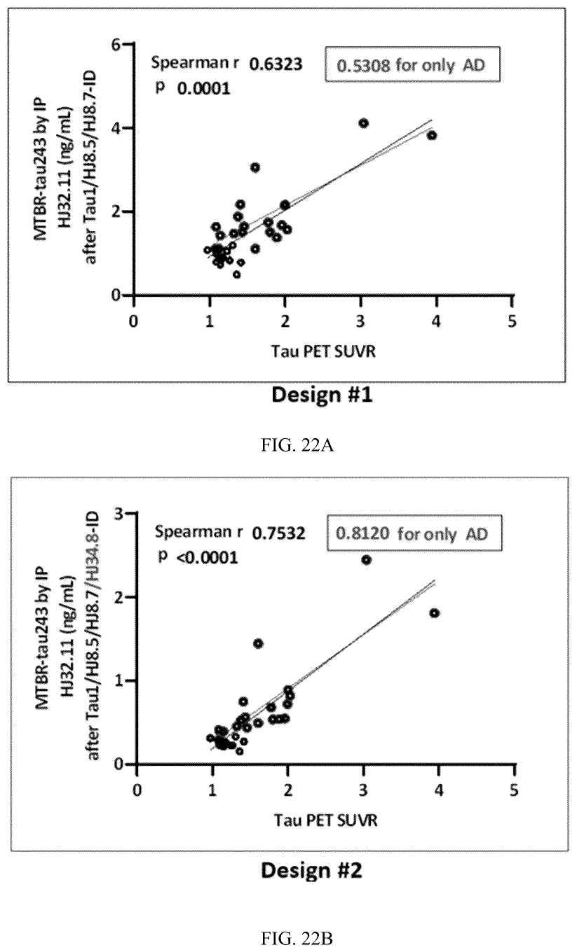

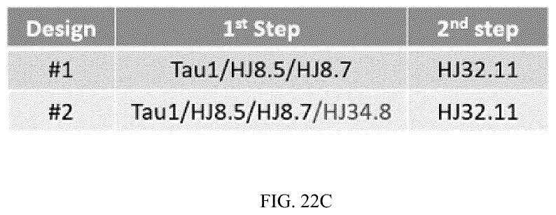

The two immunoprecipitation and/or immunodepletion methods (with or without HJ34.8 antibody in the first immunodepletion step) were validated by correlating the results obtained using chemical extraction (CX) to isolate MTBR-tau243 fragments. FIGS. 21A-B show the correlation between the amount of MTBR-tau243 detected by chemical extraction and each of the two immunoprecipitation and/or immunodepletion methods. (FIG. 21A) Correlation of MTBR-tau243 using immunoprecipitation and/or immunodepletion of Tau1/HJ8.5/HJ8.7 and chemical extraction. (FIG. 22B) Correlation of MTBR-tau243 using immunoprecipitation and/orimmunodepletion Tau1/HJ8.5/HJ8.7/HJ34.8 and chemical extraction. As shown in FIG. 21A, Tau species precipitated without HJ34.8 did not correlate well with the CSF MTBR-tau243 prepared by the chemical extraction method. The Spearman correlation was 0.8791. The correlation improved to 0.9576 using the second method when HJ34.8 antibody was used.

FIGS. 22A-B show the correlation between the amount of MTBR-tau243 detected by the two immunoprecipitation and/or immunodepletion methods and Tau PET SUVR for 54 subjects. (FIG. 22A) Correlation of MTBR-tau243 using Tau1/HJ8.5/HJ8.7 antibodies and Tau PET SUVR. (FIG. 22B) Correlation of MTBR-tau243 using Tau1/HJ8.5/HJ8.7/HJ34.8 antibodies and Tau PET SUVR. As shown in FIG. 22A, the tau species precipitated by HJ32.11 did not correlate well with tau-PET. The Spearman correlation was 0.5308. The correlation improved to 0.7532 using the second method where HJ34.8 antibody. The improved correlation shows the criticality of the depletion using HJ34.8 in the first step immunodepletion step. The tau species precipitated by HJ34.8 did not correlate with tau-PET, so the depletion at the first step is conducted. The second immunoprecipitation using HJ32.11 showed better correlation with tau-PET.

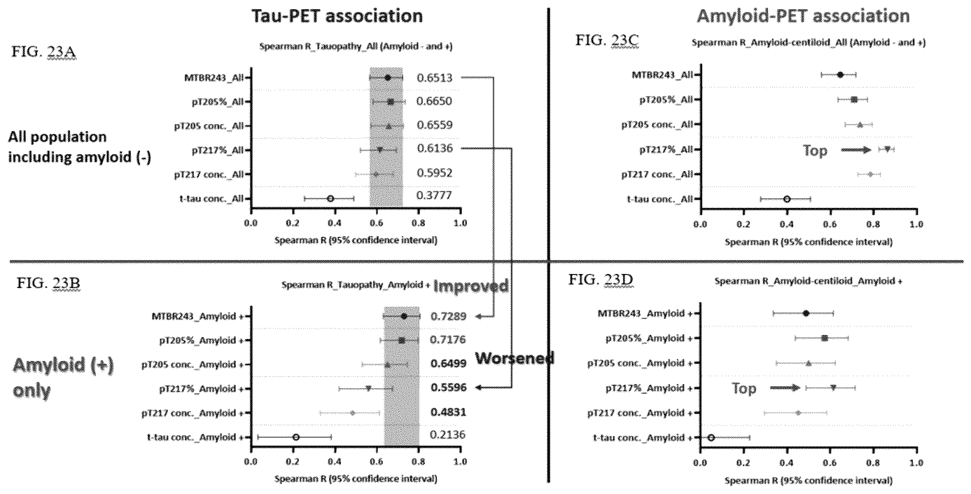

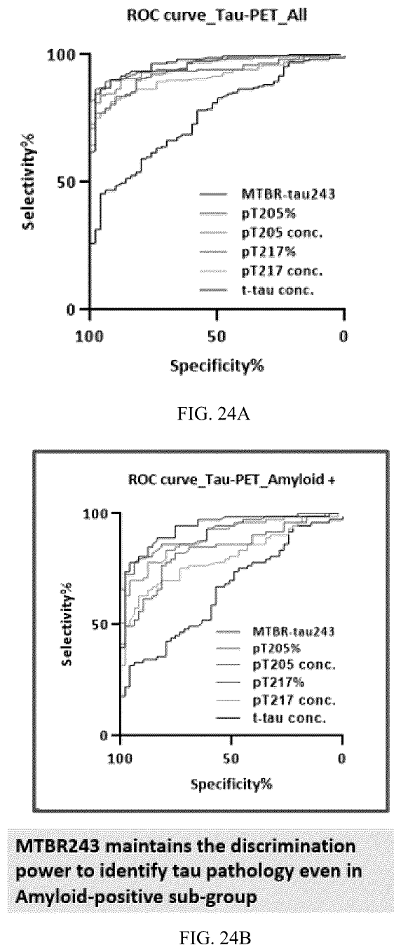

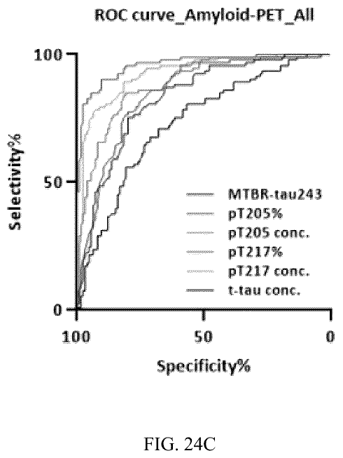

FIGS. 23A-D show MTBR-tau243 is highly associated with Tau-PET in both all and an amyloid (+) only population, but less associated with Amyloid-PET than pT217. The data was collected from 222 subjects. pT205 is the intermediate characteristic (between MTBR-tau243 and pT217) to recapitulate both amyloid and tau pathology. FIGS. 24A-C show MTBR-tau243 maintains the discrimination power to identify tau-PET positivity in even Amyloid-positive sub-group as well as all participants. These data show that cleaved fragments of tau containing MTBR-tau243 directly correlate with the Tau-PET status.

### Example 3

### MTBR-Tau 243 Detection in Plasma as Compared to CSF

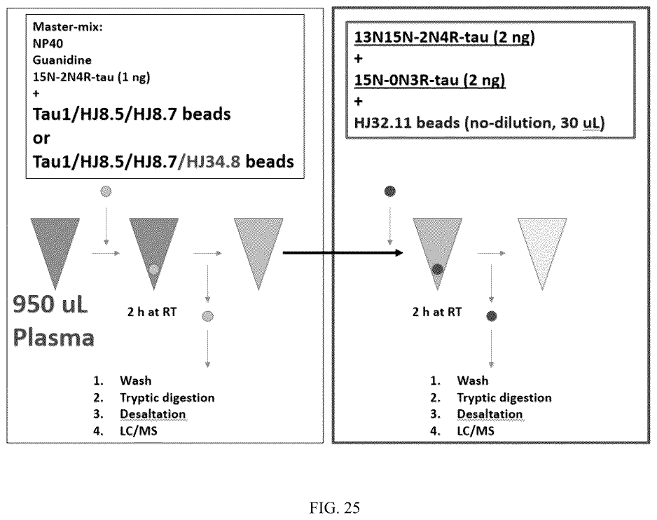

This example describes methods of detecting MTBR-tau243 fragments such as MTBR-tau243-256V and MTBR-tau243-256V (dN) in plasma vs. CSF, and demonstrates that these fragments can be used to detect tangles. As shown in FIG. 25, a master-mix of NP40, guanidine, 1 ng of ¹⁵N-2N4R-tau internal standard), and anti-tau antibody beads Tau1/HJ8.5/HJ8.7 (or Tau1/HJ8.5/HJ8.7/HJ34.8) was prepared. The master-mix was added to 950 μL of plasma samples and incubated for 2 hours at room temperature for affinity depletion (immunodepletion) or immunoprecipitation of MTBR-tau243 resulting in a first immunodepleted precipitate including the antibody bead and plasma solution. The first immunodepleted precipitate was washed and subjected to trypsin for trypsin digestion. The digested sample was desalted by C18 toptip (Glygen) and analyzed by LC/MS. The remaining plasma sample was mixed with a second mixture of 2 ng of ¹³N¹⁵N-2N4R-tau (internal standard), 2 ng of ¹⁵N-0N3R-tau (internal standard), and 30 μL of HJ32.11 antibody beads and incubated for 2 hours at room temperature for affinity depletion (immunodepletion) or immunoprecipitation of MTBR-tau243 resulting in a second immunodepleted precipitate including the HJ32.11 antibody bead. The second immunodepleted precipitate was washed and subjected to trypsin for trypsin digestion. The digested sample was desalted by C18 toptip (Glygen) and analyzed by LC/MS.

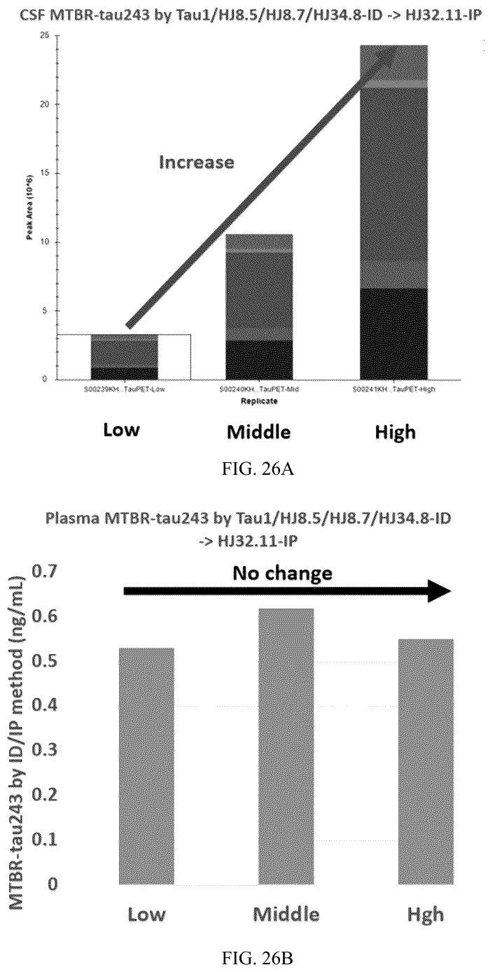

The MTBR-tau243 vs. Tau-PET status obtained from CSF and plasma from subjects are shown in FIGS. 26A-B. FIGS. 26A-B show cleaved fragments of tau containing MTBR-tau243 correlate with the Tau-PET status (low, medium, high) in CSF (FIG. 26A) but not in plasma (FIG. 26B). As shown in FIG. 26B, the two-step immunodepletion and/or immunoprecipitation method did not separate distinguish between low, middle, and high Tau-PET as well as in CSF (FIG. 26A). The two step immunoprecipitation method using tryptic digestion method did not correlate to Tau-PET status in plasma.

Detecting MTBR-Tau243 in Plasma Vs. CSF and the Use of ArgC Endopeptidase

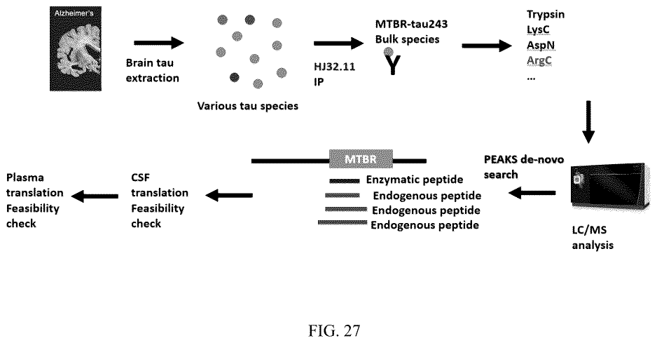

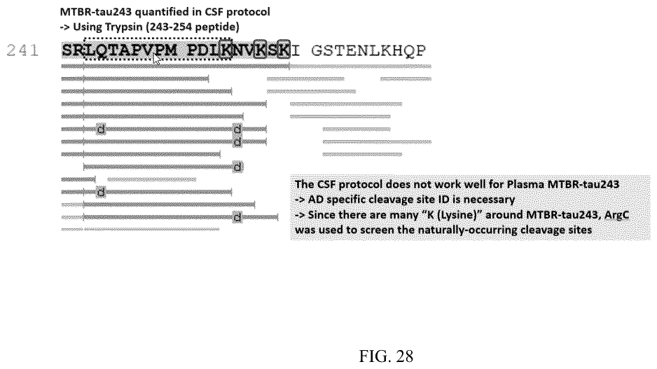

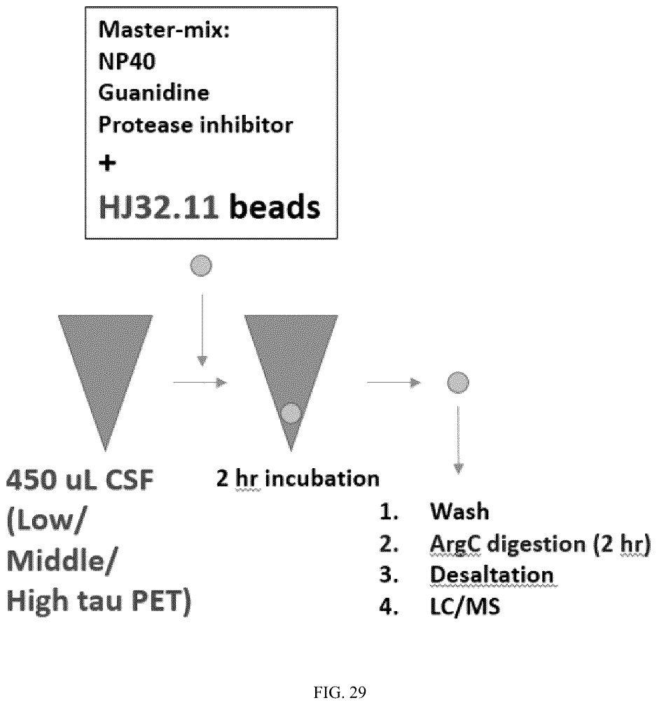

As described above, a two-step immunodepletion and/or immunoprecipitation method using trypsin digestion did not correlate to Tau-PET status in plasma. FIG. 27 shows a scheme used to determine the feasibility of detecting MTBR-tau243 species in both CSF and plasma, using tau extracted from brain. FIG. 28 shows that MTBR-tau243 peptides isolated and detected from a brain soluble fraction containing tau, using a method described above, include multiple lysine residues around the MTBR-tau243 region, which helped explain why MTBR-tau243 may not have been detected in plasma. To address this issue, it was hypothesized that digesting MTBR-tau peptides with ArgC endopeptidase could be used to screen naturally-occurring cleavage sites. One exemplary method of detecting MTBR-tau243 in CSF is shown in FIG. 29. A master-mix of NP40, guanidine, protease inhibitor, and HJ32.11 antibody beads was prepared. 450 μL of CSF samples with low, middle, and high tau PET was incubated with the master mixture for 2 hours. The HJ32.11 antibody bead was removed by immunoprecipitation. The immunoprecipitated sample was washed, digested with ArgC for 2 hours, desalted, and the resulting solution was analyzed by LC-MS. Table 14 shows CSF sample demographic for the work to identify the endogenous MTBR-tau243 species which surrogate tau-PET.

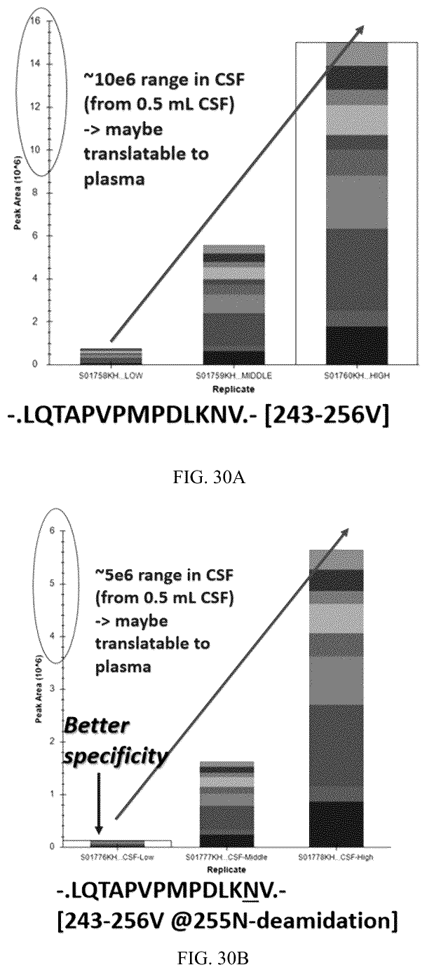

The cleaved peptide fragments detected by LC-MS are listed in Table 1. FIGS. 30A-B show the correlation of MTBR-tau243-256V (SEQ ID NO: 15) (FIG. 30A) and MTBR-tau 243-256 with @255N-deamidation (SEQ ID NO: 16) (FIG. 30B) to various Tau-PET levels in CSF. Both MTBR-tau243-256 and MTBR-tau243-256 with @255N-deamidation species increased drastically in CSF samples containing high Tau-PET.

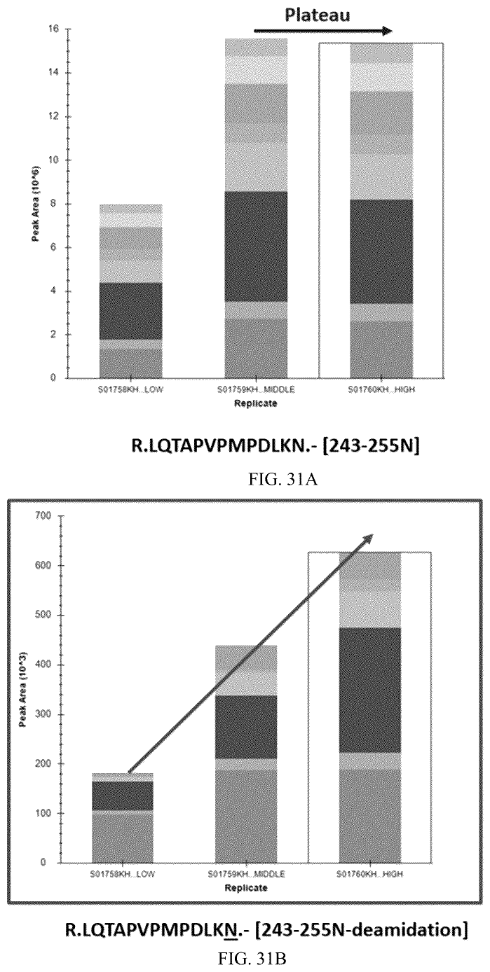

FIGS. 31A-B show the correlation of MTBR-tau243-255 (SEQ ID NO: 19) (FIG. 31A) and MTBR-tau 243-255 with @255N-deamidation (SEQ ID NO: 10) (FIG. 31B) to various levels of Tau-PET in CSF. The MTBR-tau243-255 with @255N-deamidation species showed a better specificity to low, middle, and high Tau-PET compared to the non-deamidated fragment.

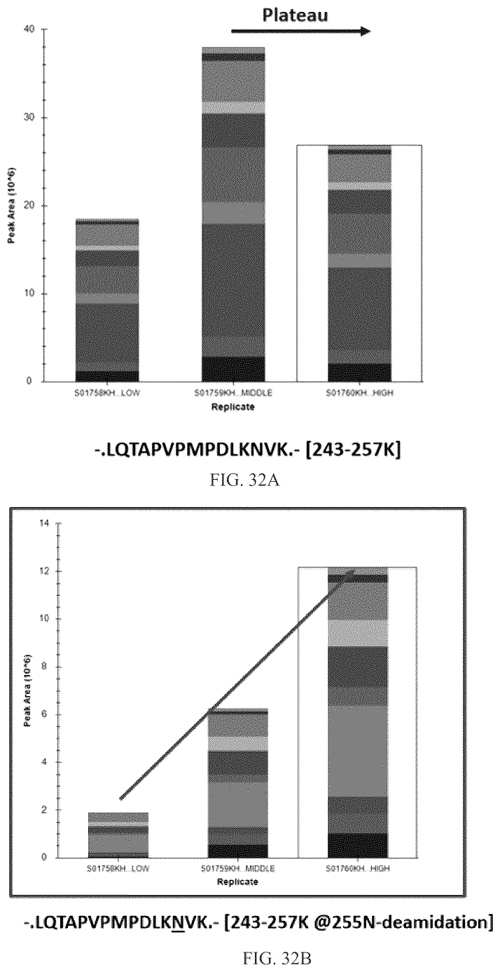

FIGS. 32A-B show the correlation of MTBR-tau243-257K (SEQ ID NO: 18) (FIG. 32A) and MTBR-tau 243-257K with @255N-deamidation (SEQ ID NO: 11) (FIG. 32B) to various levels of Tau-PET in CSF. The MTBR-tau243-257K with @255N-deamidation species showed a better specificity to low, middle, and high Tau-PET compared to the non-deamidated fragment.

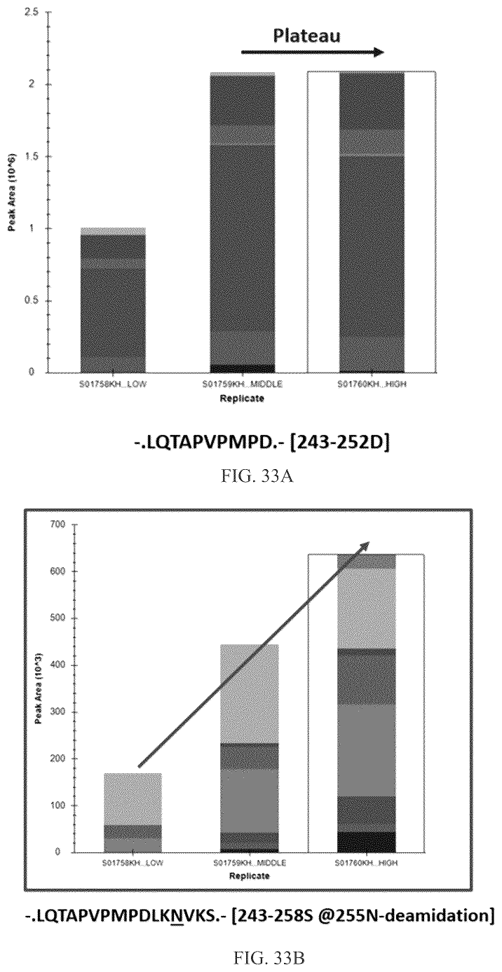

FIGS. 33A-B show the correlation of MTBR-tau243-252D (SEQ ID NO: 13) (FIG. 33A) and MTBR-tau 243-258S with @255N-deamidation (SEQ ID NO: 14) (FIG. 33B) to various levels of Tau-PET in CSF. The MTBR-tau243-258S with @255N-deamidation species showed a better specificity to low, middle, and high Tau-PET compared to the non-deamidated fragment.

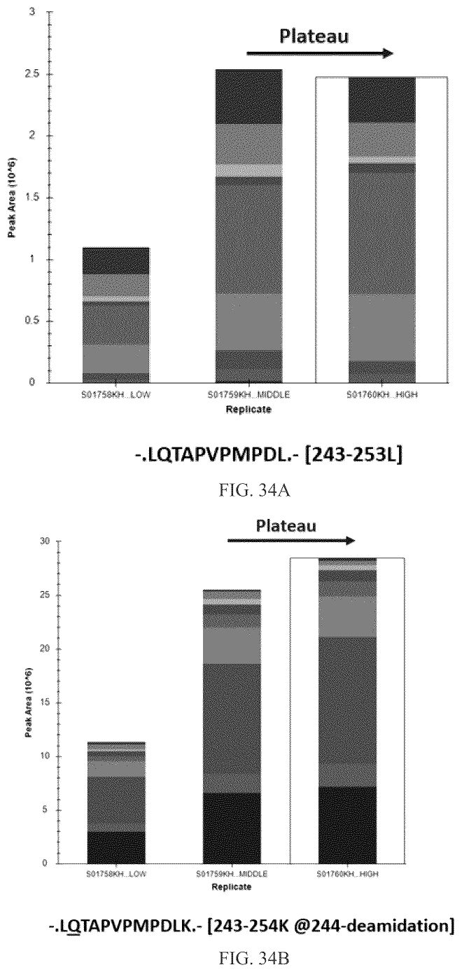

FIGS. 34A-B show the correlation of MTBR-tau243-253L (SEQ ID NO: 12) (FIG. 34A) and MTBR-tau 243-254K with @244-deamidation (SEQ ID NO: 14) (FIG. 34B) to Tau-PET in CSF. Both species plateau from middle to high Tau-PET.

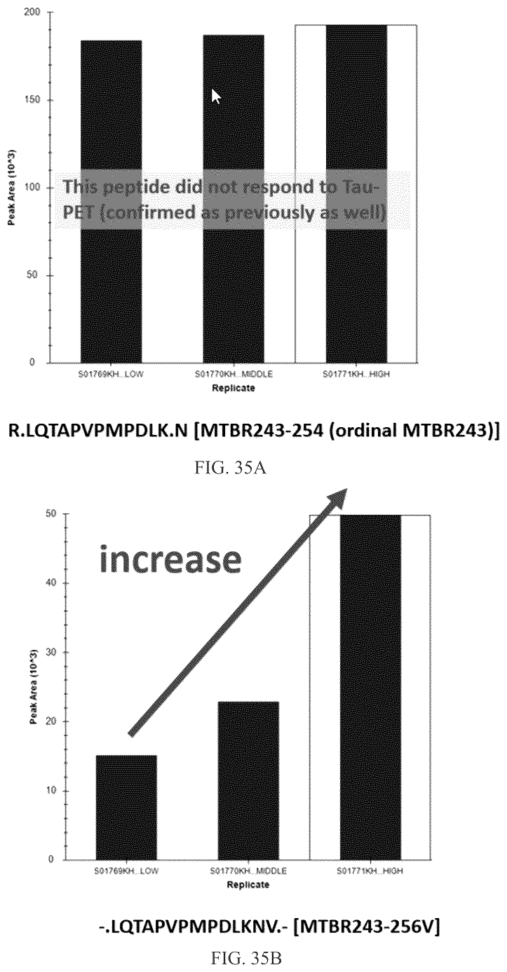

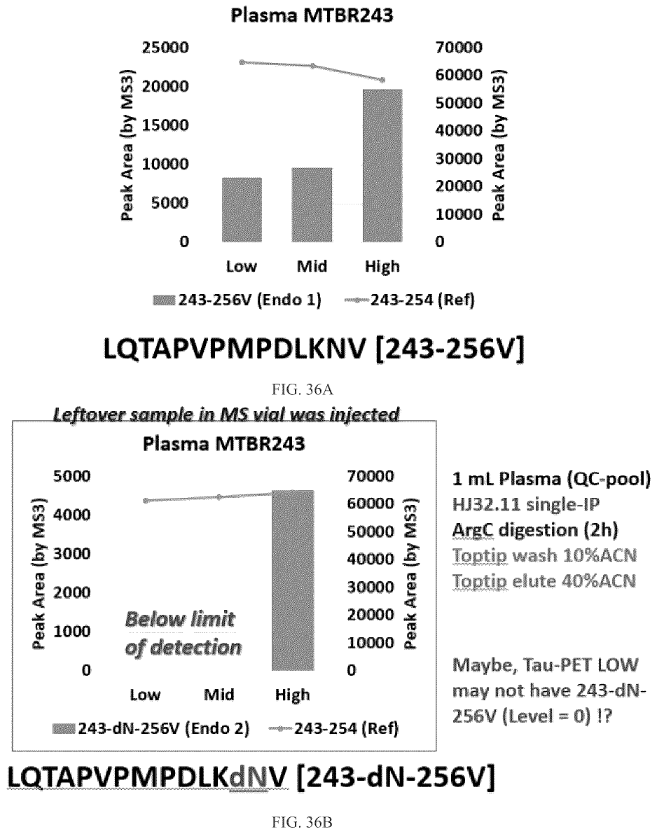

In comparison to the CSF results, FIGS. 35A-B show the correlation of MTBR-tau243-253L (SEQ ID NO: 12) (FIG. 35A) and MTBR-tau 243-256V with @244-deamidation (SEQ ID NO: 20) (FIG. 35B) to various levels of Tau-PET in plasma. The MTBR-tau243-254 species did not correspond to Tau-PET. In contrast, the MTBR-tau243-256V fragment correlated well with increasing levels of Tau-PET.

### Detecting Tau Peptides in Plasma

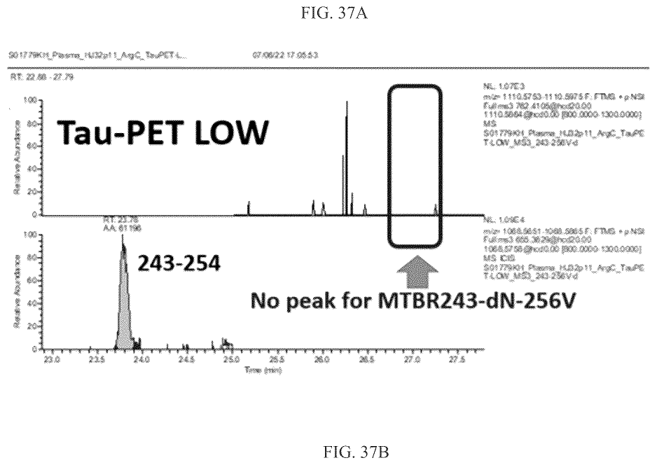

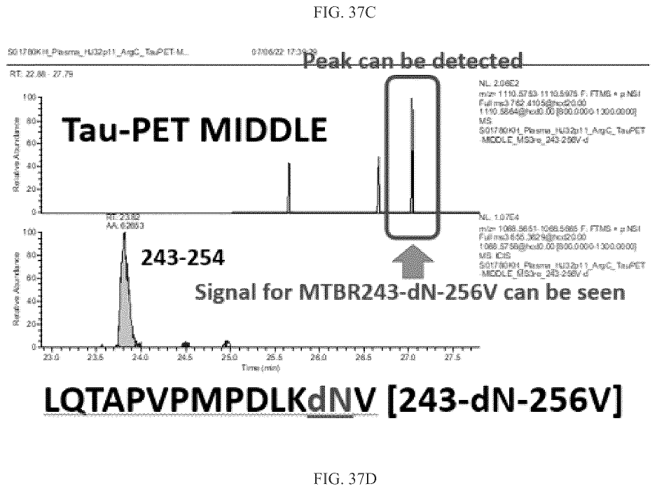

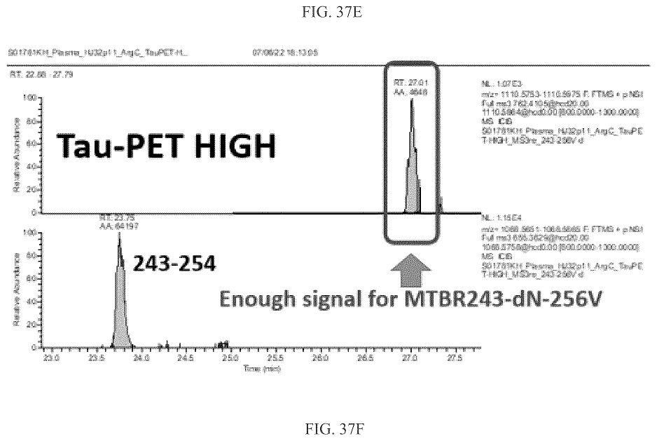

A one-step immunoprecipitation method described herein was used on plasma collected from subjects. FIG. 36A combines the plots of MTBR-tau243-254 (SEQ ID NO: 21) and MTBR-tau243-256V (SEQ ID NO: 15). Notably, 243-256V @255N-deamidation (SEQ ID NO: 16) was only detected for high Tau-PET and not for low and medium Tau-PET (FIG. 36B). FIGS. 37A-F show LC/MS data corresponding to MTBR-tau243-256V @255N-deamidation (SEQ ID NO: 16) levels as a function of Tau-PET status.

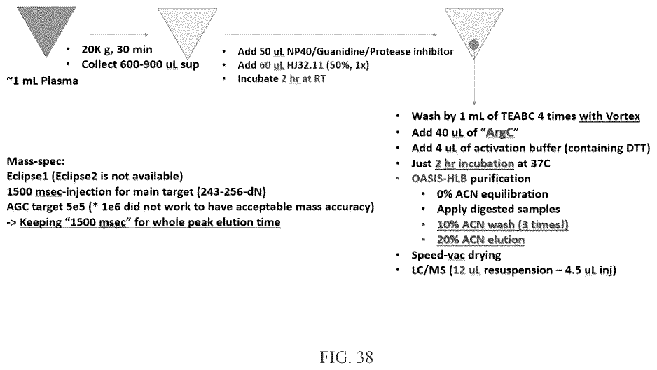

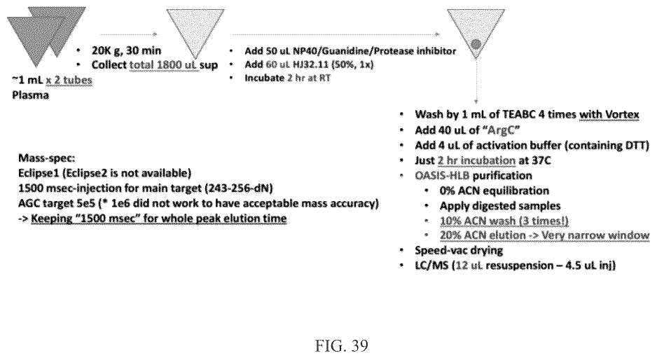

To detect deamidated fragments like 243-256V @255N-deamidation (SEQ ID 16), the immunoprecipitation method using ArgC was further modified. FIGS. 38 and 39 show two exemplary methods. In the method shown in FIG. 38, about 1 mL of plasma was collected and placed in either a single tube or multiple tubes. The tubes were placed in a centrifuge and spun at 20,000 G for 30 minutes. 600-800 μL of the supernatant was collected. 50 μL of NP40/guanidine/protease inhibitor and 60 μL of HJ32.11 antibody (50%, 1×) were added to the supernatant and incubated for 2 hours at room temperature. The sample was immunoprecipitated to remove the HJ32.11 antibody from the supernatant. The HJ32.11 antibody was washed and vortexed four times using TEABC buffer. 40 μL of ArgC endopeptidase and 4 μL of activation buffer containing DTT were added to the washed mixture and incubated for 2 hours at 37° C. to digest the samples. Next, the mixture was purified by passing it through an OASIS-HLB purification system. The system was equilibrated with 0% acetonitrile. The digested samples were injected to the OASIS-HLB purification system. The digested samples were washed with 10% acetonitrile three times and eluted using a 20% acetonitrile solution. The separated samples were collected and dried using speed-vacuum drying. The dried samples were resuspended and injected into an Eclipse™ LC/MS.

In the method shown in FIG. 39, plasma (1800 μL) was mixed with 20 μL of solution containing 0.05 fmol/μL each of stable isotope-labelled 243-256 with N-deamidation (MTBR-tau243-dN) and 243-256 (MTBR-tau243) as internal standards. Then, tau species were immunoprecipitated with the antibody HJ32.11 at room temperature for 2 hours. All immunoprecipitated tau species were digested by Arg-C and desaltated by OASIS-HLB (Waters). The resulting tau peptides were analyzed by nanoAcquity ultra-performance LC system (Waters) coupled to Orbitrap Tribrid Eclipse mass spectrometer (Thermo Scientific) operating in parallel reaction monitoring mode

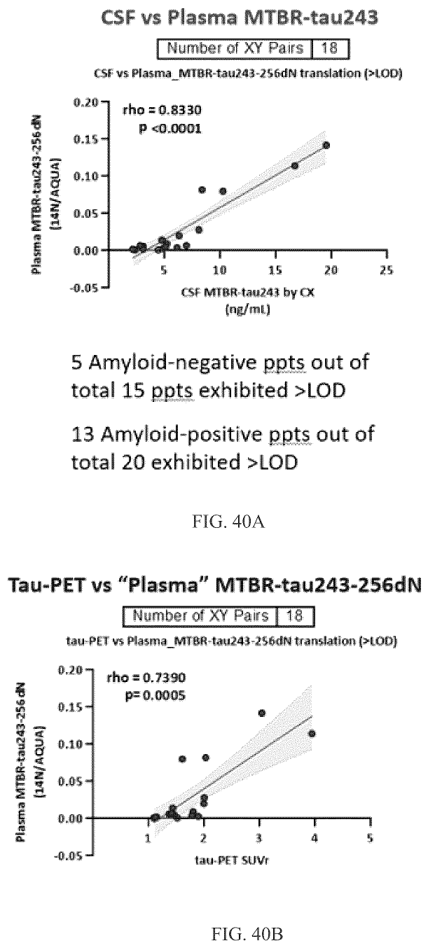

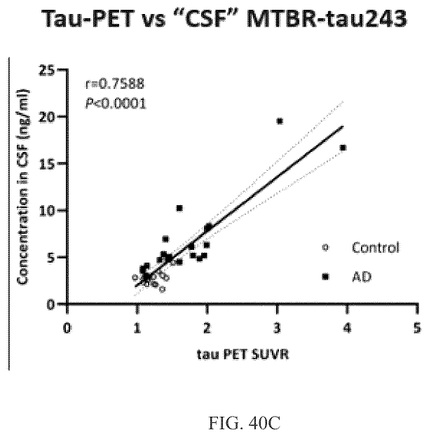

FIGS. 40A-C show correlations of 243-256V @255N-deamidation (SEQ ID NO: 16) obtained from plasma and MTBR-tau243 from CSF (using chemical extraction) (FIG. 40A), Tau-PET and plasma 243-256V @255N-deamidation (FIG. 40B), and Tau-PET and CSF MTBR-tau243 (FIG. 40 C). The modified immunoprecipitation method using ArgC digestion was able to detect 243-256V @255N-deamidation (SEQ ID NO: 16) in plasma. It was found that control subjects (amyloid-negative) did not show any peak of 243-256V @255N-deamidation (SEQ ID NO: 16). This endogenous species is very specific to tau pathology, so zero value is obtained for control participants as shown in FIG. 40C. Notably, plasma and CSF 243-256V @255N-deamidation (SEQ ID NO: 16) was highly correlated with Tau-PET status (FIGS. 40A-B).

A summary of endogenously cleaved peptides, their detection in CSF and/or plasma, and ability to stage Alzheimer's disease is provided in Table 19.

### Plasma MTBR-Tau243-256 (dN) for AD Staging

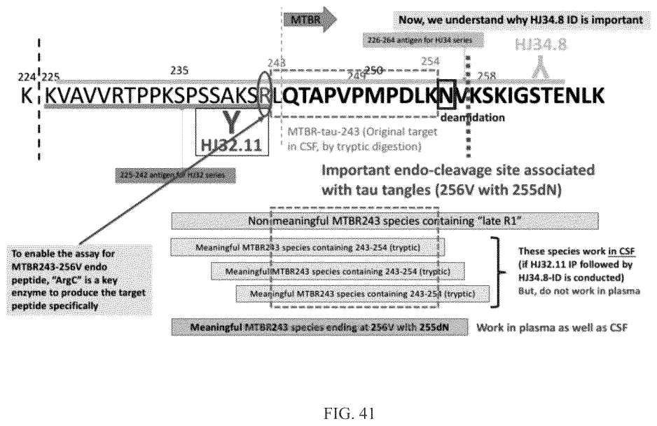

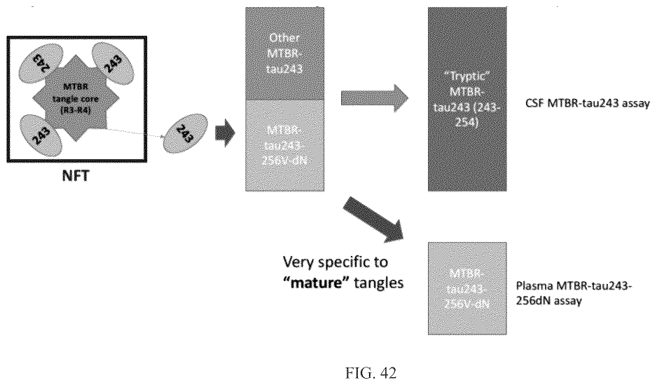

FIG. 41 shows the amino acid sequence of the region around the 243-256V @255N-deamidation (SEQ ID NO: 16) peptide and how various anti-tau antibodies bind to the region. The figure helps explain how the use of HJ32.11 and HJ34.8 in a method of detection in plasma as compared to CSF provides meaningful information about the presence of tangles. FIG. 42 shows a hypothesis as to why plasma 243-256V @255N-deamidation (SEQ ID NO: 16) increases drastically in plasma after AD onset (CDR ≥1), as compared to MTBR-tau243-254 in CSF

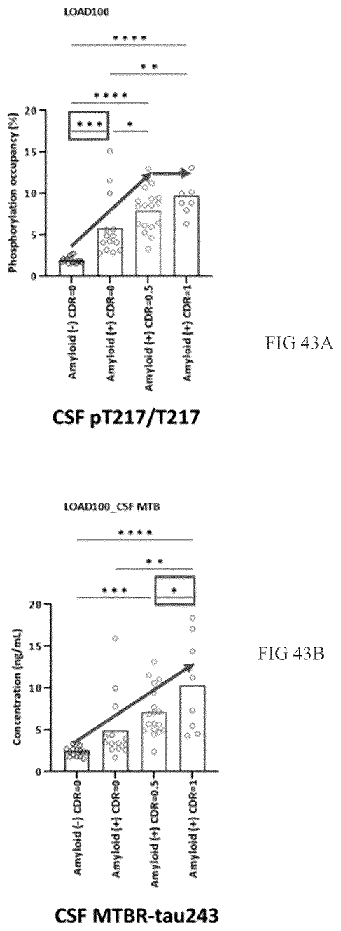

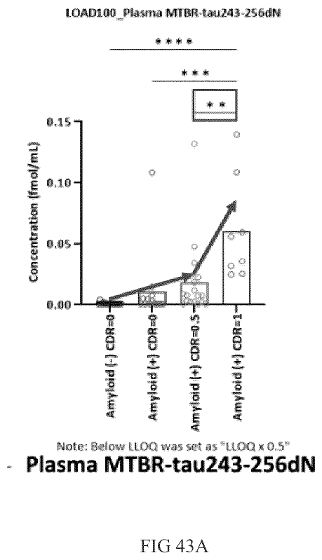

As shown in FIGS. 43A-C, 243-256V @255N-deamidation in plasma is indicative of the stage of Alzheimer's disease in a subject. FIG. 43A shows CSF pT217/T217 plateaus at a clinical dementia rating (CDR) score of 0.5 or greater. FIG. 43B shows CSF MTBR-tau243 steadily increases with an increase in CDR score. Notably, FIG. 43C shows plasma MTBR-tau243-256 (dN) levels increase steeply at CDR scores greater than 0.5. MTBR-tau243-256V @255N-deamidation levels increase drastically when the CDR score is greater than or equal to 1.

### Example 4

MTBR-Tau 212-221 Correlates with Tau-PET Levels in Plasma

Referring back to FIG. 19, FIG. 19 shows the different MTBR-tau fragments isolated by HJ34.8 antibody bead in the first immunodepletion step and by anti-tau antibody HJ32.11 bead in the second immunoprecipitation step. Very short MTBR-tau243 fragments showed affinity for HJ32.11 antibodies and exhibited a high association with tau tangles. Notably, MTBR-tau212-221 fragments also showed affinity for HJ32.11 antibodies.

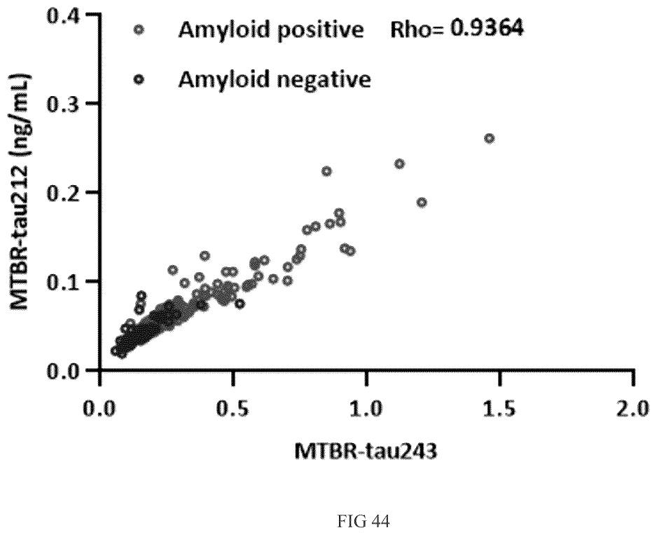

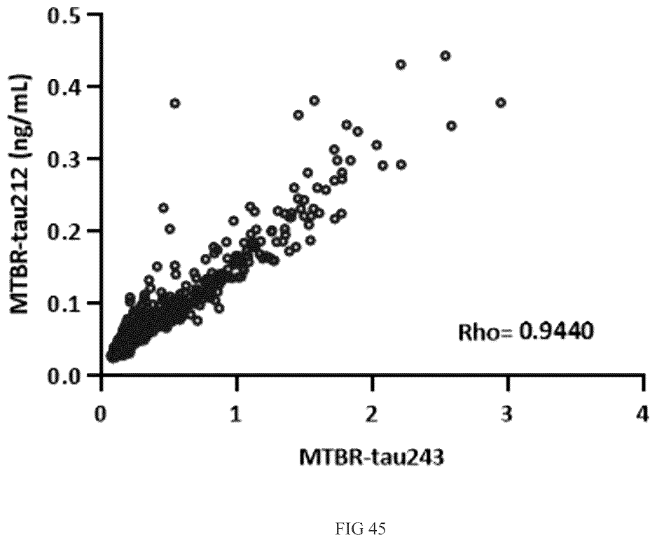

This example shows that MTBR-tau212-221 fragments are also highly associated with tau tangles. FIG. 44 and Table 16 show that MTBR-tau212-221 was highly correlated with MTBR-tau243 in plasma samples obtained from a Knight ADRC cohort. FIG. 45 and Table 17 show that MTBR-tau212-221 was highly correlated with MTBR-tau243 in plasma samples obtained from a BioFINDER 2 cohort.

### REFERENCES

The entirety of the disclosures of the following references, and any other reference disclosed herein, are incorporated herein by reference.
1. Ossenkoppele, R., van der Kant, R. & Hansson, O. Tau biomarkers in Alzheimer's disease: towards implementation in clinical practice and trials. The Lancet Neurology 21, 726-734 (2022).
2. Smith, R., Wibom, M., Pawlik, D., Englund, E. & Hansson, O. Correlation of In Vivo [18F]Flortaucipir With Postmortem Alzheimer Disease Tau Pathology. JAMA Neurol 76, 310-317 (2019).
3. Fleisher, A. S. et al. Positron Emission Tomography Imaging With [18F]flortaucipir and Postmortem Assessment of Alzheimer Disease Neuropathologic Changes. JAMA Neurol 77, 829-839 (2020).
4. Smith, R. et al. 18F-AV-1451 tau PET imaging correlates strongly with tau neuropathology in MAPT mutation carriers. Brain 139, 2372-2379 (2016).
5. Soleimani-Meigooni, D. N. et al. 18F-flortaucipir PET to autopsy comparisons in Alzheimer's disease and other neurodegenerative diseases. Brain 143, 3477-3494 (2020).
6. Lowe, V. J. et al. Tau-positron emission tomography correlates with neuropathology findings. Alzheimers Dement 16, 561-571 (2020).
7. Ossenkoppele, R. et al. Amyloid and tau PET-positive cognitively unimpaired individuals are at high risk for future cognitive decline. Nat Med 28, 2381-2387 (2022).
8. Smith, R. et al. Tau-PET is superior to phospho-tau when predicting cognitive decline in symptomatic AD patients. Alzheimer's & Dementia doi: 10.1002/alz.12875. Online ahead of print.
9. Sato, C. et al. Tau Kinetics in Neurons and the Human Central Nervous System. Neuron 97, 1284-1298.e7 (2018).
10. Cicognola, C. et al. Novel tau fragments in cerebrospinal fluid: relation to tangle pathology and cognitive decline in Alzheimer's disease. Acta Neuropathol 137, 279-296 (2019).
11. Barthélemy, N. R., Horie, K., Sato, C. & Bateman, R. J. Blood plasma phosphorylated-tau isoforms track CNS change in Alzheimer's disease. Journal of Experimental Medicine 217, (2020).
12. Janelidze, S. et al. Plasma P-tau181 in Alzheimer's disease: relationship to other biomarkers, differential diagnosis, neuropathology and longitudinal progression to Alzheimer's dementia. Nature Medicine 26, 379-386 (2020).
13. Palmqvist, S. et al. Discriminative Accuracy of Plasma Phospho-tau217 for Alzheimer Disease vs Other Neurodegenerative Disorders. JAMA 324, 772-781 (2020).
14. Thijssen, E. H. et al. Diagnostic value of plasma phosphorylated tau181 in Alzheimer's disease and frontotemporal lobar degeneration. Nature Medicine 26, 387-397 (2020).
15. Karikari, T. K. et al. Blood phosphorylated tau 181 as a biomarker for Alzheimer's disease: a diagnostic performance and prediction modelling study using data from four prospective cohorts. The Lancet Neurology 19, 422-433 (2020).
16. O'Connor, A. et al. Plasma phospho-tau181 in presymptomatic and symptomatic familial Alzheimer's disease: a longitudinal cohort study. Mol Psychiatry 26, 5967-5976 (2021).
17. Ashton, N. J. et al. Plasma p-tau231: a new biomarker for incipient Alzheimer's disease pathology. Acta Neuropathol 141, 709-724 (2021).
18. Therriault, J. et al. Association of Phosphorylated Tau Biomarkers With Amyloid Positron Emission Tomography vs Tau Positron Emission Tomography. JAMA Neurology (2022) doi: 10.1001/jamaneurol.2022.4485.
19. Mattsson-Carlgren, N. et al. Soluble P-tau217 reflects amyloid and tau pathology and mediates the association of amyloid with tau. EMBO Mol Med 13, (2021).
20. Salvadó, G. et al. Specific associations between plasma biomarkers and postmortem amyloid plaque and tau tangle loads. EMBO Molecular Medicine e2463 (2023).
21. Barthélemy, N. R. et al. A soluble phosphorylated tau signature links tau, amyloid and the evolution of stages of dominantly inherited Alzheimer's disease. Nature Medicine 26, 398-407 (2020).
22. Milà-Alomà, M. et al. Plasma p-tau231 and p-tau217 as state markers of amyloid-β pathology in preclinical Alzheimer's disease. Nat Med 1-5 (2022) doi: 10.1038/s41591-022-01925-w.
23. Mattsson-Carlgren, N. et al. Aβ deposition is associated with increases in soluble and phosphorylated tau that precede a positive Tau PET in Alzheimer's disease. Science Advances 6, eaaz2387 (2020).
24. Janelidze, S. et al. Associations of Plasma Phospho-Tau217 Levels With Tau Positron Emission Tomography in Early Alzheimer Disease. JAMA Neurology 78, 149-156 (2021).
25. Pontecorvo, M. J. et al. Association of Donanemab Treatment With Exploratory Plasma Biomarkers in Early Symptomatic Alzheimer Disease: A Secondary Analysis of the TRAILBLAZER-ALZ Randomized Trial. Clinical JAMA Neurology (2022) doi: 10.1001/jamaneurol.2022.3392.
26. Budd Haeberlein, S. et al. Two Randomized Phase 3 Studies of Aducanumab in Early Alzheimer's Disease. J Prev Alzheimers Dis 9, 197-210 (2022).
27. Salloway, S. et al. A trial of gantenerumab or solanezumab in dominantly inherited Alzheimer's disease. Nat Med 27, 1187-1196 (2021).
28. van Dyck, C. H. et al. Lecanemab in Early Alzheimer's Disease. N Engl J Med (2022) doi: 10.1056/NEJMoa2212948.
29. Barthélemy, N. R. et al. CSF tau phosphorylation occupancies at T217 and T205 represent improved biomarkers of amyloid and tau pathology in Alzheimer's disease. Nat Aging 3, 391-401 (2023).
30. Schelle, J. et al. Prevention of tau increase in cerebrospinal fluid of APP transgenic mice suggests downstream effect of BACE1 inhibition. Alzheimer's & Dementia 13, 701-709 (2017).
31. Maia, L. F. et al. Changes in Amyloid-β and Tau in the Cerebrospinal Fluid of Transgenic Mice Overexpressing Amyloid Precursor Protein. Science Translational Medicine 5, 194re2-194re2 (2013).
32. Kaeser, S. A. et al. CSF p-tau increase in response to Aβ-type and Danish-type cerebral amyloidosis and in the absence of neurofibrillary tangles. Acta Neuropathol 143, 287-290 (2022).
33. Horie, K., Barthélemy, N. R., Sato, C. & Bateman, R. J. CSF tau microtubule binding region identifies tau tangle and clinical stages of Alzheimer's disease. Brain 144, 515-527 (2021).
34. Roberts, M. et al. Pre-clinical characterisation of E2814, a high-affinity antibody targeting the microtubule-binding repeat domain of tau for passive immunotherapy in Alzheimer's disease. acta neuropathol commun 8, 13 (2020).
35. Taniguchi-Watanabe, S. et al. Biochemical classification of tauopathies by immunoblot, protein sequence and mass spectrometric analyses of sarkosyl-insoluble and trypsin-resistant tau. Acta Neuropathol. 131, 267-280 (2016).
36. Shi, Y. et al. Structure-based classification of tauopathies. Nature 598, 359-363 (2021).
37. Fitzpatrick, A. W. P. et al. Cryo-EM structures of tau filaments from Alzheimer's disease. Nature 547, 185-190 (2017).
38. Janelidze, S. et al. Head-to-head comparison of 10 plasma phospho-tau assays in prodromal Alzheimer's disease. Brain awac333 (2022) doi: 10.1093/brain/awac333.
39. Binette, A. P. et al. Amyloid-associated increases in soluble tau is a key driver in accumulation of tau aggregates and cognitive decline in early Alzheimer. 2022.01.07.22268767 Preprint at https://doi.org/10.1101/2022.01.07.22268767 (2022).
40. Folstein, M. F., Folstein, S. E. & McHugh, P. R. “Mini-mental state”: A practical method for grading the cognitive state of patients for the clinician. Journal of Psychiatric Research 12, 189-198 (1975).
41. Janelidze, S. et al. Cerebrospinal fluid p-tau217 performs better than p-tau181 as a biomarker of Alzheimer's disease. Nature Communications 11, 1-12 (2020).
42. Ashton, N. J. et al. Differential roles of Aβ42/40, p-tau231 and p-tau217 for Alzheimer's trial selection and disease monitoring. Nat Med 1-8 (2022) doi: 10.1038/s41591-022-02074-w.
43. Mielke, M. M. et al. Comparison of Plasma Phosphorylated Tau Species With Amyloid and Tau Positron Emission Tomography, Neurodegeneration, Vascular Pathology, and Cognitive Outcomes. JAMA Neurol 78, 1108-1117 (2021).
44. Moscoso, A. et al. Time course of phosphorylated-tau181 in blood across the Alzheimer's disease spectrum. Brain 144, 325-339 (2021).
45. Salvadó, G. et al. Specific associations between plasma biomarkers and post-mortem amyloid plaque and neurofibrillary tau tangle loads. 2022.08.22.22279052 Preprint at https://doi.org/10.1101/2022.08.22.22279052 (2022).
46. Murray, M. E. et al. Global neuropathologic severity of Alzheimer's disease and locus coeruleus vulnerability influences plasma phosphorylated tau levels. Molecular Neurodegeneration 17, 85 (2022).
47. Lindquist, S. G. et al. Alzheimer disease-like clinical phenotype in a family with FTDP-17 caused by a MAPT R406W mutation. European Journal of Neurology 15, 377-385 (2008).
48. de Silva, R. et al. An immunohistochemical study of cases of sporadic and inherited frontotemporal lobar degeneration using 3R- and 4R-specific tau monoclonal antibodies. Acta Neuropathol 111, 329-340 (2006).
49. Wolters, E. E. et al. [18F]Flortaucipir PET Across Various MAPT Mutations in Presymptomatic and Symptomatic Carriers. Neurology 97, e1017-e1030 (2021).
50. Tsai, R. M. et al. 18F-flortaucipir (AV-1451) tau PET in frontotemporal dementia syndromes. Alzheimer's Research & Therapy 11, 13 (2019).
51. Jones, D. T. et al. In vivo 18F-AV-1451 tau PET signal in MAPT mutation carriers varies by expected tau isoforms. Neurology 90, e947-e954 (2018).
52. Ashton, N. J. et al. Differential roles of Aβ42/40, p-tau231 and p-tau217 for Alzheimer's trial selection and disease monitoring. Nat Med 28, 2555-2562 (2022).
53. Jack, C. R. et al. A/T/N: An unbiased descriptive classification scheme for Alzheimer disease biomarkers. Neurology 87, 539-547 (2016).
54. Therriault, J. et al. Association of Phosphorylated Tau Biomarkers With Amyloid Positron Emission Tomography vs Tau Positron Emission Tomography. JAMA Neurology 80, 188-199 (2023).
55. Clark, C. M. et al. Cerebral PET with florbetapir compared with neuropathology at autopsy for detection of neuritic amyloid-β plaques: a prospective cohort study. Lancet Neurol 11, 669-678 (2012).
56. Sabri, O. et al. Florbetaben PET imaging to detect amyloid beta plaques in Alzheimer's disease: phase 3 study. Alzheimers Dement 11, 964-974 (2015).
57. Ikonomovic, M. D. et al. Post-mortem histopathology underlying β-amyloid PET imaging following flutemetamol F 18 injection. Acta Neuropathol Commun 4, 130 (2016).
58. Marquié, M. et al. Validating novel tau positron emission tomography tracer [F-18]-AV-1451 (T807) on postmortem brain tissue. Ann Neurol 78, 787-800 (2015).
59. Dubois, B. et al. Clinical diagnosis of Alzheimer's disease: recommendations of the International Working Group. The Lancet Neurology 20, 484-496 (2021).
60. Morris, J. C. The Clinical Dementia Rating (CDR): Current version and scoring rules. Neurology 43, 2412-a (1993).
61. Morris, J. C. et al. The Uniform Data Set (UDS): Clinical and Cognitive Variables and Descriptive Data From Alzheimer Disease Centers. Alzheimer Disease & Associated Disorders 20, 210-216 (2006).
62. Volluz, K. E. et al. Correspondence of CSF biomarkers measured by Lumipulse assays with amyloid PET. Alzheimer's & Dementia 17, e051085 (2021).
63. Bourgeat, P. et al. Implementing the centiloid transformation for 11C-PiB and B-amyloid 18F-PET tracers using CapAIBL. NeuroImage 183, 387-393 (2018).
64. Cho, H. et al. In vivo cortical spreading pattern of tau and amyloid in the Alzheimer disease spectrum. Annals of Neurology 80, 247-258 (2016).
65. Leuzy, A. et al. A multicenter comparison of [18F]flortaucipir, [18F]RO948, and [18F]MK6240 tau PET tracers to detect a common target ROI for differential diagnosis. Eur J Nucl Med Mol Imaging 48, 2295-2305 (2021).
66. Chen, C. D. et al. Comparing Tau PET Visual Interpretation with Tau PET Quantification, Cerebrospinal Fluid Biomarkers, and Longitudinal Clinical Assessment. Journal of Alzheimer's Disease 93, 765-777 (2023).

1. An anti-MTBR tau antibody comprising:
(a) an L1, L2, and L3 from a light chain variable region from the sequence set forth in SEQ ID NO: 6, and an H1, H2, and H3 from a heavy chain variable region from the sequence set forth in SEQ ID NO: 7;
(b) an L1, L2, and L3 from a light chain variable region sequence from the sequence set forth in SEQ ID NO: 2, and an H1, H2, and H3 from a heavy chain variable region from the sequence set forth in SEQ ID NO: 3;
(c) an L1, L2, and L3 from a light chain variable region from the sequence set forth in SEQ ID NO: 4, and an H1, H2, and H3 from a heavy chain variable region from the sequence set forth in SEQ ID NO: 5; or
(d) an L1, L2, and L3 from a light chain variable region from the sequence set forth in SEQ ID NO: 8, and an H1, H2, and H3 from a heavy chain variable region from the sequence set forth in SEQ ID NO: 9.

2. The anti-MTBR tau antibody of claim 1, comprising
(a) a light chain variable region from the sequence set forth in SEQ ID NO: 6 and a heavy chain variable region from the sequence set forth in SEQ ID NO: 7;
(b) a light chain variable region from the sequence set forth in SEQ ID NO: 2 and a heavy chain variable region from the sequence set forth in SEQ ID NO: 3
(c) a light chain variable region from the sequence set forth in SEQ ID NO: 4 and a heavy chain variable region from the sequence set forth in SEQ ID NO: 5; or
(d) a light chain variable region from the sequence set forth in SEQ ID NO: 8 and a heavy chain variable region from the sequence set forth in SEQ ID NO: 9.

3. (canceled)

4. (canceled)

5. A method of detecting insoluble tau aggregates in a biological sample, comprising:
(a) purifying endogenously cleaved fragments of tau by contacting the biological sample with an epitope-binding agent that specifically binds an epitope within amino acids 235-242 of SEQ ID NO: 1, without first contacting the endogenously cleaved fragments of tau in vitro with a protease;
(b) contacting the purified endogenously cleaved fragments of tau with an Arg-C endopeptidase to obtain proteolytic MTBR-tau243 peptides comprising amino acids 243-254 of SEQ ID NO: 1; and
(c) detecting the proteolytic MTBR-tau243 peptides by performing liquid chromatography-mass spectrometry (LC/MS) or an immunoassay, wherein detecting the proteolytic MTBR-tau243 peptides is indicative of insoluble tau aggregates in the biological sample,
wherein the biological sample is a plasma sample or cerebrospinal fluid (CSF) sample,
optionally wherein the method further comprises one or more of detecting and quantifying one or more of amyloid beta, N-terminal tau, mid-domain tau, post-translational modifications of tau, and/or an ApoE isoform in the biological sample.

6. (canceled)

7. The method of claim 5, wherein the proteolytic MTBR-tau243 peptides comprise one or more of MTBR-tau243-254, MTBR-tau243-256V, MTBR-tau243-256V (dN), and MTBR-tau212-221.

8. (canceled)

9. (canceled)

10. (canceled)

11. (canceled)

12. (canceled)

13. (canceled)

14. A method of detecting insoluble tau aggregates in a cerebrospinal fluid (CSF) sample, comprising
(a) performing affinity depletion on a CSF sample by contacting the sample with affinity depletion agents comprising one or more epitope-binding agents that each binds to one of N-terminal tau, mid-domain tau, and long-MTBR tau, but not to an antigen within amino acids 235-256 of SEQ ID NO: 1, wherein the CSF sample comprises cleaved fragments of tau, to obtain a depleted sample and an enriched sample, wherein the depleted sample comprises N-terminal tau, mid-domain tau, and long-MTBR tau, and wherein the enriched sample is enriched for endogenously cleaved fragments of tau comprising amino acids 235-256 of SEQ ID NO: 1 (endogenous MTBR-tau243 peptides);
(b) performing immunoprecipitation on the enriched sample by contacting the enriched sample with an immunoprecipitation agent comprising an epitope-binding agent that binds to endogenous MTBR-tau243 peptides, to obtain a purified sample;
(c) contacting the endogenous MTBR-tau243 peptides in the purified sample with a protease to obtain a sample comprising proteolytic MTBR-tau243 peptides; and
(d) detecting the proteolytic MTBR-tau243 peptides by performing liquid chromatography-mass spectrometry (LC/MS) or an immunoassay, wherein detecting the proteolytic MTBR-tau243 peptides is indicative of insoluble tau aggregates in the CSF sample,
optionally wherein the method further comprises detecting and/or quantifying amyloid beta, detecting and/or quantifying N-terminal tau, detecting and/or quantifying mid-domain tau, detecting and/or quantifying post-translational modifications of tau, or identifying an ApoE isoform in the biological or CSF sample.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method of detecting insoluble tau aggregates in a plasma sample, comprising
(a) performing affinity depletion on a plasma sample by contacting the sample with affinity depletion agents comprising one or more epitope-binding agents that each binds to one of N-terminal tau, mid-domain tau, and long-MTBR tau, but not to an antigen within amino acids 235-256 of SEQ ID NO: 1, wherein the plasma sample comprises cleaved fragments of tau, to obtain a depleted sample and an enriched sample, wherein the depleted sample comprises N-terminal tau, mid-domain tau, and long-MTBR tau, and wherein the enriched sample is enriched for endogenously cleaved fragments of tau comprising amino acids 235-254 of SEQ ID NO: 1 (endogenous MTBR-tau243 peptides);
(b) performing immunoprecipitation on the enriched sample by contacting the enriched sample with an immunoprecipitation agent comprising an epitope-binding agent that binds to endogenous MTBR-tau243 peptides, to obtain a purified sample;
(c) contacting the endogenous MTBR-tau243 peptides with a protease comprising an Arg-C endopeptidase to obtain a sample comprising proteolytic MTBR-tau243 peptides; and
(d) detecting the proteolytic MTBR-tau243 peptides by performing liquid chromatography-mass spectrometry (LC/MS) or an immunoassay, wherein detecting one or more of MTBR-tau243-256V, MTBR-tau243-256V (dN) and MTBR-tau212-221 is indicative of insoluble tau aggregates in the plasma sample,
optionally wherein the method further comprises detecting and/or quantifying amyloid beta, detecting and/or quantifying N-terminal tau, detecting and/or quantifying mid-domain tau, detecting and/or quantifying post-translational modifications of tau, or identifying an ApoE isoform in the biological or CSF sample.

31. (canceled)

32. The method of claim 30, wherein the affinity depletion agents comprise: an epitope-binding agent that specifically binds to an epitope within amino acids 1 to 243 of SEQ ID NO: 1; a first epitope-binding agent that specifically binds to an epitope within amino acids 1 to 103 of tau-441, and a second epitope-binding agent that specifically binds to an epitope within amino acids 104 to 243 of SEQ ID NO: 1; or, the first epitope-binding agent, the second epitope-binding agent, and a third epitope-binding agent that specifically binds to an epitope within amino acids 257-264 of SEQ ID NO: 1.

33. (canceled)

34. (canceled)

35. (canceled)

36. (canceled)

37. (canceled)

38. (canceled)

39. (canceled)

40. (canceled)

41. (canceled)

42. (canceled)

43. A method of detecting an Alzheimer disease (AD)-related pathology in a subject, comprising detecting insoluble tau aggregates in a CSF or plasma sample according to the method of claim 5, wherein the CSF or plasma sample is from the subject, and wherein detecting insoluble aggregates of tau is indicative of the AD-related pathology in the subject.

44. (canceled)

45. (canceled)

46. A method of detecting Alzheimer disease (AD)-related tau deposition in a brain of a subject, comprising detecting insoluble tau aggregates in a CSF or plasma sample according to the method of claim 5, wherein the CSF or plasma sample is from the subject, and wherein detecting insoluble aggregates of tau is indicative of AD-related tau deposition in the brain of the subject.

47. A method of diagnosing Alzheimer's disease in a subject, comprising
(a) detecting insoluble tau aggregates in a CSF or plasma sample according to the method of claim 5, wherein the CSF or plasma sample is from the subject; and
(b) diagnosing Alzheimer's disease when the amount of a proteolytic MTBR-tau243 peptide detected differs by about 1.50 or more above the mean of a healthy control population, wherein σ is the standard deviation defined by the normal distribution measured in a control population that is amyloid negative as measured by PET imaging and/or Aβ42/40 measurement in CSF.

48. A method of measuring Alzheimer disease (AD) progression in a subject, comprising:
(a) detecting insoluble tau aggregates in a first and a second CSF or plasma sample according to the method of claim 5; and
(b) calculating a difference between amounts of a proteolytic MTBR-tau243 peptide in the second sample and the first sample, wherein a statistically significant increase in the amount of the proteolytic MTBR-tau243 peptide in the second sample as compared to the first sample indicates progression of the subject's Alzheimer's disease.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. A method of treating a tauopathy in a subject in need thereof, comprising
(a) detecting insoluble tau aggregates in a CSF or plasma sample according to the method of claim 5, wherein the CSF or plasma sample is from the subject; and
(b) administering to the subject a treatment that alters tau pathology, wherein the amount of a proteolytic MTBR-tau243 peptide detected differs by about 1.5σ or more above the mean of a healthy control population, wherein σ is the standard deviation defined by the normal distribution measured in a control population that does not have clinical signs or symptoms of a tauopathy and is amyloid negative as measured by PET imaging and/or Aβ42/40 measurement in CSF, and wherein the amount of the proteolytic MTBR-tau243 peptide is indicative of a tauopathy.

61. A method of treating a tauopathy in a subject in need thereof, comprising administering to the subject a treatment that alters tau pathology, wherein the subject has been identified as having an amount of a proteolytic MTBR-tau243 peptide detected according to the method of claim 5, wherein the amount differs by about 1.5σ or more above the mean of a healthy control population, wherein σ is the standard deviation defined by the normal distribution measured in a control population that does not have clinical signs or symptoms of a tauopathy and is amyloid negative as measured by PET imaging and/or Aβ42/40 measurement in CSF, and wherein the amount of the proteolytic MTBR-tau243 peptide is indicative of a tauopathy.

62. (canceled)

63. The method of claim 60, wherein the treatment is selected from the group consisting of lecanemab, donanemab, AADvac1, ACI-3024, ACI-35, APNmAb005, ASN51, AZP2006, BIIB076, BIIB080, BIIB 113, Bepranemab, Dasatinib+Quercetin, E2814, Epothilone D, Gosuranemab, JNJ-63733657, LM™, LY3372689, Lu AF87908, MK-2214, NIO752, OLX-07010, PNT001, PRX005, RG7345, Rember™, Semorinemab, TPI 287, Tideglusib, Tilavonemab, Zagotenemab, an anti-tau monoclonal antibody, an anti-tau anti-sense oligonucleotide, an anti-tau small interfering RNA, an tau production inhibitor, and a tau active vaccine.

64. The method of claim 63, wherein the treatment is selected from the group consisting of anti-Aβ antibodies, anti-tau antibodies, anti-TREM2 antibodies, TREM2 agonists, gamma-secretase inhibitors, beta-secretase inhibitors, a kinase inhibitor, a phosphatase activator, a vaccine, and a tau protein aggregation inhibitor.

65. The method of claim 64, wherein
(a) the kinase inhibitor is an inhibitor of a thousand-and-one amino acid kinase (TAOK), CDK, GSK-3β, MARK, CDK5, or Fyn;
(b) the phosphatase activator increases the activity of protein phosphatase 2A;
(c) the vaccine is CAD106 or AF20513; or
(d) the anti-Aβ antibody is aducanumab or another anti-amyloid antibody that removes plaques.

66. (canceled)

67. (canceled)

68. (canceled)

69. A kit, comprising the antibody of claim 1, and instructions for use.

70. A kit, comprising a means for purifying tau from a biological sample, and instructions for use according to the method of claim 5.

71. A kit, comprising a means for purifying tau from a biological sample, and instructions for use according to the method of claim 14.


SEQUENCE LISTING (22 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 132 aa, Mus musculus
MESQTQVFLSLLLWVSGTCGNIMITQSPSSLAVSAGEKVTMSCKSSQSVLYSSDRKNYLAWYQQKPGQSPKLLIFWASTRESGVPDRFTGSGSGTDFTLTINSVQAEDLAVYYCHQYLSSRTFGGGTKLEIK

SEQ ID NO: 3 - protein, 137 aa, Mus musculus
MKCSWIIFFLMAVVTGVNSEVQLQQSGAELVKPGASVKLSCTASGFNIKDYFMHWVKQRTEQGLEWIGRIDPEDDETKYAPKFQGKATITADTSSNTVYLHLSSLTSEDTAVYYCARDGSTYSMDYWGQGTSVTVSS

SEQ ID NO: 4 - protein, 131 aa, Mus musculus
MKLPVRLLVLMFWIPASSSDVLMTQTPLSLSVSLGDQVSISCRSSQSIEHSNGNTYLEWYLQKPGQSPKLLIYKVSNRFSGVPDRFSGSGSGTDFTLKISRVEAEDLGVYYCFQGLHVPYTFGGGTKLEIK

SEQ ID NO: 5 - protein, 132 aa, Mus musculus
MKCSWIIFFLMAVVTGVNSEVQLQQSGAELVKPGASVKLSCTASGFNIYDYFIHWVKQRTEQGLEWIGRIDPEDGEIKFAPKFQGKATITADTSSNTAYLQLSSLTSEDTAVYYCGCDFDYWGQGTTLTVSS

SEQ ID NO: 6 - protein, 130 aa, Mus musculus
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGLHVPYTFGGGTKLEI

SEQ ID NO: 7 - protein, 132 aa, Mus musculus
MKCSWIIFFLMAVVTGVNSEVQLQQSGAELVKPGASVRLSCTGSGLNIKDYFIHWVKQRTKQGLEWIGRIDPEDDEIKYAPKFQGKATITADTSSNTAYLHLISLTSEDTAVYYCGWDFNYWGQGTTLTVSS

SEQ ID NO: 8 - protein, 132 aa, Mus musculus; identical to SEQ ID NO: 2

SEQ ID NO: 9 - protein, 137 aa, Mus musculus
MKCSWIIFFLMAVVTGVNSEVQLQQSGAELVKPGASVKLSCTASGFNIKDYFMHWVKQRTEQGLEWIGRIDPEDDETKYDPKFQGKATITADTSFNTVYLHLSSLTSEDTAVYYCARDGSTYSMDYWGQGTSVTVSS

SEQ ID NO: 10 - protein, 13 aa, Homo sapiens
  MOD_RES 13: note = deamidated
LQTAPVPMPDLKN

SEQ ID NO: 11 - protein, 15 aa, Homo sapiens
  MOD_RES 13: note = deamidated
LQTAPVPMPDLKNVK

SEQ ID NO: 12 - protein, 11 aa, Homo sapiens
LQTAPVPMPDL

SEQ ID NO: 13 - protein, 11 aa, Homo sapiens
DLQTAPVPMPD

SEQ ID NO: 14 - protein, 16 aa, Homo sapiens
  MOD_RES 13: note = deamidated
LQTAPVPMPDLKNVKS

SEQ ID NO: 15 - protein, 14 aa, Homo sapiens
LQTAPVPMPDLKNV

SEQ ID NO: 16 - protein, 14 aa, Homo sapiens
  MOD_RES 13: note = deamidated
LQTAPVPMPDLKNV

SEQ ID NO: 17 - protein, 10 aa, Homo sapiens
TPSLPTPPTR

SEQ ID NO: 18 - protein, 15 aa, Homo sapiens
LQTAPVPMPDLKNVK

SEQ ID NO: 19 - protein, 13 aa, Homo sapiens
LQTAPVPMPDLKN

SEQ ID NO: 20 - protein, 12 aa, Homo sapiens
  MOD_RES 2: note = deamidated
LQTAPVPMPDLK

SEQ ID NO: 21 - protein, 12 aa, Homo sapiens
LQTAPVPMPDLK

SEQ ID NO: 22 - protein, 30 aa, Homo sapiens
SRLQTAPVPMPDLKNVKSKIGSTENLKHQP